(19) United States
(12) Patent Application Publication
(10) Pub. No.: US 2026/0315879 A1
(43) Pub. Date: Oct. 15, 2026

(54) THIN FILM SENSOR ARRAYS FOR SURFACES HAVING NON-ZERO GAUSSIAN CURVATURE

(71) Applicant: GRIPFZN INC., Ann Arbor, MI (US)

(72) Inventor: Mason Ferlic, Ann Arbor, MI (US)

(21) Appl. No.: 19/641,079

(22) Filed: Apr. 7, 2026

Related U.S. Application Data
(60) Provisional application No. 63/919,753, filed on Nov. 18, 2025.
(60) Provisional application No. 63/785,830, filed on Apr. 9, 2025.

(51) Int. Cl. A63B 43/00; A63B 24/00; A63B 69/00; G06F 3/041; A63B 102/18

(52) U.S. Cl. CPC A63B 43/004; A63B 24/0062; A63B 43/007; A63B 69/0002; G06F 3/04144; G06F 3/04164; A63B 2102/18

(57) ABSTRACT

Systems and devices are described that include a conformal sensor device having a thin-film substrate having a plurality of discrete regions and a plurality of patterned cutouts; a sensor array comprising a plurality of sensor elements disposed in a non-Cartesian pattern on the thin-film substrate, wherein the thin-film substrate having the plurality of sensor elements disposed thereon is configured to conform to a surface having non-zero Gaussian curvature to provide substantially uniform sensing at the surface; and an integrated circuit electrically coupled to the plurality of sensor elements, wherein the integrated circuit is configured to analyze signals from the sensor array to determine one or more interactions with the surface.

### CROSS-REFERENCE TO RELATED APPLICATIONS

This application claims the priority benefit of U.S. Provisional Patent Application Ser. No. 63/785,830, filed Apr. 9, 2025, and claims the priority benefit of U.S. Provisional Patent Application Ser. No. 63,919,753, filed Nov. 18, 2025, the contents of each of which are herein incorporated by reference in their entirety.

### INCORPORATION BY REFERENCE

All publications and patent applications mentioned in this specification are herein incorporated by reference in their entirety, as if each individual publication or patent application was specifically and individually indicated to be incorporated by reference in its entirety.

### TECHNICAL FIELD

This disclosure relates generally to the field of sensorized objects and more specifically to the field of sensing input on curved surfaces.

### BACKGROUND

Sensor arrays are traditionally manufactured using an additive printing technique involving printing layers of conductive and dielectric inks on a thin foil substrate to achieve desired electrical and mechanical properties. The printing process for electronic sensors on thin foil is typically more cost-effective than traditional manufacturing processes for electronics. Thin foil substrates allow sensors to be flexible, enabling them to conform to surfaces and shapes where traditional rigid electronics cannot. However, while conventional sensor architectures can tolerate a flexible substrate (such as polyimide film), the flexible substrate will have low elasticity i.e., low stretch to ensure functional stability and durability of the resulting electronic devices. Further, substrates with low elasticity ensure that the dimensions of the printed electronics remain stable under mechanical stress or changes in environmental conditions. High elasticity substrates could stretch or deform, causing misalignment of the printed layers or components, which could lead to malfunctioning of the electronic devices and/or stretching and breakage of conductive traces that form the circuits of the device.

### SUMMARY

There is a need for new and useful systems and methods for design and manufacture of a printed sensor array on an inelastic substrate that can conform to non-developable shapes, such as spheres, ellipsoids, hyperbolic paraboloids, or other surfaces having non-zero Gaussian curvature or double curvature.

The described embodiments, relate to improved sensor devices and associated methods for thin film sensor array(s) for input (e.g., grip) assessment on curved surfaces. In some embodiments, the curved surfaces provide a thin-film sensor array designed to conform to surfaces with non-zero Gaussian curvature, addressing the limitations of traditional planar sensor designs. The sensor array may feature discrete petal-shaped regions and patterned cutouts strategically positioned to enable localized strain relief and mechanical conformity to complex curved geometries. These petal-shaped regions may be engineered to overlap and interleave, ensuring uniform sensing coverage while minimizing distortion and buckling during application. The patterned cutouts may include auxetic structures and edge slits that enhance substrate flexibility and prevent mechanical failure under stress.

In some aspects, the techniques described herein relate to a conformal sensor device including: a thin-film substrate having a plurality of discrete regions and a plurality of patterned cutouts; a sensor array including a plurality of sensor elements disposed in a non-Cartesian pattern on the thin-film substrate, wherein the thin-film substrate having the plurality of sensor elements disposed thereon is configured to conform to a surface being substantially curved to provide substantially uniform sensing at the surface; and an integrated circuit electrically coupled to the plurality of sensor elements, wherein the integrated circuit is configured to analyze signals from the sensor array to determine one or more interactions with the surface.

In some aspects, the techniques described herein relate to a conformal sensor device, wherein the integrated circuit includes a processor and a memory including computer-readable instructions configured to trigger communication about the analyzed signals or interactions with the surface.

In some aspects, the techniques described herein relate to a conformal sensor device, wherein: the thin-film substrate includes a plurality of petal-shaped segments, and the plurality of patterned cutouts are wave-shaped or sawtooth-shaped along a perimeter of the plurality of petal-shaped segments.

In some aspects, the techniques described herein relate to a conformal sensor device, wherein: the plurality of petal-shaped segments include active sensor areas and inactive sensor areas, and bending portions of the thin-film substrate to converge a distal end of each petal-shaped segment at an apex of the surface results in overlapping portions of the thin-film substrate to provide uniform sensing on the surface.

In some aspects, the techniques described herein relate to a conformal sensor device, wherein: the substantially curved surface is a first surface having non-zero Gaussian curvature for receiving the thin-film substrate thereon; and the conformal sensor device further includes a second surface as an outer layer of a material surrounding the thin-film substrate conformed to the first surface.

In some aspects, the techniques described herein relate to a conformal sensor device, wherein the plurality of sensor elements is configured to measure at least one of: a pressure, a vibration, a shock, an acceleration, a stress, a force, a touch, humidity, a presence, damage, radiation, and temperature.

In some aspects, the techniques described herein relate to a conformal sensor device, wherein the plurality of patterned cutouts include a cutout depth to minimize buckling deformation of the thin-film substrate and to minimize sensor performance degradation of one or more of the plurality of sensor elements when the conformal sensor device is curved about the surface.

In some aspects, the techniques described herein relate to a conformal sensor device, wherein the thin-film substrate includes a plurality of layers and at least a portion of the layers include cutouts in the plurality of patterned cutouts that enable adhesion of one layer to another of the plurality of layers.

In some aspects, the techniques described herein relate to a conformal sensor device, wherein the conformal sensor device is a grip assessment device configured to measure grip characteristics of a user interacting with the substantially curved surface.

In some aspects, the techniques described herein relate to a conformal sensor device, wherein the sensor array and a plurality of electrical trace lines coupling the sensor array to a ring bus are printed onto portions of one or more layers of the thin-film substrate, and a backing layer of the thin-film substrate includes adhesive for coupling the thin-film substrate to a portion of the surface.

In some aspects, the techniques described herein relate to a conformal sensor device, wherein the thin-film substrate further includes: a plurality of petal-shaped segments, and a plurality of electrical trace lines coupling each of the plurality of sensor elements to a ring bus, the plurality of electrical trace lines being arranged substantially along a central meridian of each of the plurality of petal-shaped segments and within a predetermined clearance range defined from a perimeter of each respective petal-shaped segment.

In some aspects, the techniques described herein relate to a conformal sensor device, wherein at least a portion of the thin-film substrate is composed of heat shrink material.

In some aspects, the techniques described herein relate to a conformal sensor device, wherein each of the plurality of sensor elements includes a discrete electrical element printed on the thin-film substrate having a non-square shape including at least one of: a triangular shape, an oval shape, a rectangular shape, or a polygonal shape other than rectangular.

In some aspects, the techniques described herein relate to a conformal sensor device, wherein each of the plurality of sensor elements includes a first discrete sensor element having a rectangular shape printed on the thin-film substrate and a second discrete sensor element having a rectangular shape printed on the thin-film substrate overlaid on the first discrete sensor element and oriented 90 degrees offset from a centerline of the first discrete sensor element.

In some aspects, the techniques described herein relate to a conformal sensor device, wherein the sensor array is configured to distinguish finger orientation and identify digits of a user gripping at least a portion of the surface.

In some aspects, the techniques described herein relate to a conformal sensor device, wherein the surface includes an equatorial plane, wherein the sensor array extends radially outward from the equatorial plane, and wherein the integrated circuit is positioned at the equatorial plane and electrically coupled to the plurality of sensor elements of the sensor array.

In some aspects, the techniques described herein relate to a conformal sensor device, wherein the sensor elements are arranged asymmetrically with variable densities to provide increased sensing resolution in targeted sensing areas where interaction frequency is expected to be higher relative to other areas of the surface.

In some aspects, the techniques described herein relate to a conformal sensor device, wherein the integrated circuit is further configured to analyze signals from the sensor array to determine one or more of: a grip of a user gripping a portion of the substantially curved surface; a movement or series of movements of the substantially curved surface; and interactions with the portion of the substantially curved surface over a time period.

In some aspects, the techniques described herein relate to an electronic device configured for installation in a ball, the electronic device including: a thin-film substrate having a hub and a plurality of protrusions each extending from one of a plurality of bridges coupled to a portion of the hub; a sensor array including a plurality of sensor elements disposed in a pattern over the plurality of protrusions, the plurality of sensor elements being divided amongst the plurality of protrusions and interconnected by a plurality of electrical trace lines emanating from each sensor element to another sensor element or to at least one ring bus; and an integrated circuit electrically coupled to the at least one ring bus, the integrated circuit including a processor and a memory including computer-readable instructions configured to analyze measurements from signals from the sensor array and trigger communication about the analyzed measurements.

In some aspects, the techniques described herein relate to an electronic device, wherein the plurality of protrusions each include a first edge having a first set of cutouts extending along a first side of a periphery of the respective protrusion in the plurality of protrusions to a second edge having a second set of cutouts extending along a second side of the periphery of the respective protrusion.

In some aspects, the techniques described herein relate to an electronic device, wherein the first set of cutouts and the second set of cutouts are wave-shaped or sawtooth-shaped.

In some aspects, the techniques described herein relate to an electronic device, wherein the first set of cutouts and the second set of cutouts in the thin-film substrate are selected, based on a diameter or radius of curvature of the ball, with a cutout depth to minimize strain deformation of the thin-film substrate when curved about the ball.

In some aspects, the techniques described herein relate to an electronic device, wherein each of the plurality of protrusions is petal-shaped and includes a petal periphery with the first set of cutouts and the second set of cutouts in the thin-film substrate.

In some aspects, the techniques described herein relate to an electronic device, wherein each of the protrusions is configured to cover a portion of a curved surface of a hemispherical shell when deformed about the respective bridges and coupled to the portion of the curved surface such that a distal end of each of the protrusions substantially covers an apex of the hemispherical shell.

In some aspects, the techniques described herein relate to an electronic device, wherein the thin-film substrate includes active sensor areas and inactive regions, and bending at the respective bridges to substantially cover the apex results in overlapping portions of the plurality of protrusions to provide substantially uniform sensing across a surface of the ball when the electronic device is installed in the ball.

In some aspects, the techniques described herein relate to an electronic device, wherein each of the protrusions is configured to cover a portion of a curved surface of a prolate spheroid shape when deformed about the respective bridges and coupled to the portion of the curved surface such that a distal end of each of the protrusions substantially covers an apex of the prolate spheroid shape.

In some aspects, the techniques described herein relate to an electronic device, wherein the plurality of sensor elements is configured to measure at least one of: a pressure, a vibration, a shock, an acceleration, a stress, a force, humidity, presence, damage, radiation, and temperature.

In some aspects, the techniques described herein relate to an electronic device, further including: a second thin-film substrate having a second ring bus coupled to the integrated circuit and including a second plurality of protrusions and a second sensor array including a second plurality of sensor elements disposed in a pattern on the second plurality of protrusions, the second plurality of sensor elements being interconnected by a plurality of additional electrical trace lines emanating from each of the second plurality of sensor elements to another sensor element in the second sensor array or to the at least one ring bus, wherein the thin-film substrate is configured to wrap a first hemispherical portion of the ball, and the second thin-film substrate is configured to wrap a second hemispherical portion of the ball.

In some aspects, the techniques described herein relate to an electronic device, wherein the sensor array and the second sensor array function together through the integrated circuit to analyze interaction with a surface of the ball to determine one or more of: a grip of a user gripping the ball, a movement or series of movements of the ball, and interactions with the ball over a time period.

In some aspects, the techniques described herein relate to an electronic device, wherein the electronic device is configured to distinguish finger orientation and identify digits of a user gripping the ball.

### BRIEF DESCRIPTION OF THE DRAWINGS

FIG. 1 illustrates a sensor system diagram which supports techniques for thin film sensor array for grip assessment on curved surfaces in accordance with various aspects of the present disclosure.

FIG. 2 illustrates a monitoring system for a thin film sensor array for monitoring and assessing a grip on curved surfaces in accordance with various aspects of the present disclosure.

FIG. 3 illustrates a portion of a spherical device including thin film sensor arrays for grip assessment.

FIG. 4 illustrates a spherical sensor device for performing grip analysis on curved surfaces.

FIG. 5 illustrates an example thin film sensor array.

FIG. 6 illustrates an example sensor array having an integrated circuit.

FIG. 7 illustrates another view of the example sensor array of FIG. 6 installed on a base portion of a hemispherical shape.

FIG. 8 illustrates the example sensor array of FIG. 6 in the process of portions of the sensor array being installed.

FIG. 9 illustrates the sensor array ball 600 of FIG. 8 with additional portions of the sensor array being installed.

FIGS. 10A-10B illustrate example portions of a core for receiving the sensor arrays described herein.

FIG. 11 illustrates a hemispherical shell with a sensor array installed thereon.

FIG. 12 illustrates a baseball with one or more embedded sensor arrays for assessing a grip.

FIG. 13 illustrates example layers of a petal of the sensor arrays described herein.

FIG. 14 illustrates connector block layers of the hub described herein.

FIG. 15 illustrates an example system for a thin film sensor array for grip assessment on curved surfaces.

FIG. 16 illustrates an opposite side of the example system of FIG. 15.

FIG. 17 illustrates a sensor cross-section of an example sensor array.

FIG. 18 illustrates an example of sensor foil layers for the sensor elements described herein.

FIG. 19 illustrates an example spherical object with two sensor arrays.

FIG. 20 illustrates an example sensor foil design for the sensor arrays described herein.

FIG. 21 illustrates a flowchart illustrating a process for manufacturing a thin film sensor array for curved surface applications.

FIG. 22 illustrates an example sensor foil design for the sensor arrays described herein.

FIG. 23 illustrates an example sensor foil design for the sensor arrays described herein.

FIG. 24A-24C illustrate example pattern cutouts for the sensor foils described herein.

FIG. 25 illustrates an example pad shape for force-sensing resistor sensors described herein.

The illustrated embodiments are merely examples and are not intended to limit the disclosure. The schematics are drawn to illustrate features and concepts and are not necessarily drawn to scale.

### DETAILED DESCRIPTION

The foregoing is a summary, and thus, necessarily limited in detail. The above-mentioned aspects, as well as other aspects, features, and advantages of the present technology will now be described in connection with various embodiments. The inclusion of the following embodiments is not intended to limit the disclosure to these embodiments, but rather to enable any person skilled in the art to make and use the claimed subject matter. Other embodiments may be utilized, and modifications may be made without departing from the spirit or scope of the subject matter presented herein. Aspects of the disclosure, as described and illustrated herein, can be arranged, combined, modified, and designed in a variety of different formulations, all of which are explicitly contemplated and form part of this disclosure.

As digital sensors have become smaller, lighter, and more flexible, there is an increasing desire to incorporate sensor array technology to be applied to and/or onto curved surfaces. Conventionally, sensor arrays have been designed for planar surface applications such as measuring applied pressure or contact on a flat rectangular area. The devices and methods of manufacturing described herein are designed for application on curved, non-developable shapes/surfaces. In particular, the devices described herein are sensor arrays that conform to non-planar surfaces. The sensor arrays described herein may be arranged on a thin film to conform to non-planar surfaces of a ball, for example, to provide a uniform layout of sensor elements. In some embodiments, the sensor arrays described herein may be arranged on a thin film to conform to and/or uniformly cover an irregular geometrical surface or curvature with a uniform grid/layout of sensor elements. In some embodiments, the sensor arrays described herein may include any number of pressure sensors printed on a thin film such that the sensors are arranged to substantially and uniformly wrap a surface of an object, such as a ball (e.g., a sphere, a dome, etc.). In one non-limiting example, the sensor arrays on such an object may be programmed to measure finger grip pressure when a user grips the ball, for example, during use.

Conventional sensor arrays designed for planar surface applications are inadequate for curved surface applications having non-zero Gaussian curvature because planar sensor arrays cannot conform to non-developable surfaces without wrinkling, buckling, or tearing of the substrate. Elastic substrate approaches that attempt to address this limitation by using stretchable substrate materials are also inadequate because elastic substrates undergo dimensional changes during the conforming process that cause misalignment of printed sensor elements and electrical trace lines, compromising sensor performance and electrical integrity. The conformal sensor devices described herein address these limitations through the combination of a substantially inelastic thin-film substrate with a hub-and-petal architecture, patterned cutouts providing localized strain relief, and non-cartesian sensor element arrangements providing uniform spatial sensing resolution across the curved surface, collectively enabling accurate and reliable sensing on non-developable surfaces without the limitations of conventional planar or elastic substrate approaches.

Described herein are devices and systems including a conformal sensor device with a thin-film substrate having a plurality of discrete regions for housing sensor elements and a plurality of patterned cutouts. The devices and systems further include a sensor array with a plurality of sensor elements disposed in a non-Cartesian pattern on the thin-film substrate. In some embodiments, the sensor array including a plurality of sensor elements disposed in a non-Cartesian pattern on the thin-film substrate may refer to a sensor array including a plurality of sensor elements having sensor element geometries disposed in a non-Cartesian spatial distribution on the thin-film substrate. The thin-film substrate, with the plurality of sensor elements disposed thereon, may be conformed to a surface having non-zero Gaussian curvature to provide substantially uniform sensing at the surface. The devices and systems may further include an integrated circuit electrically coupled to the plurality of sensor elements. The integrated circuit may analyze signals from the sensor array to determine one or more interactions with the surface. In some embodiments, the sensor element geometry may include a polygonal shape other than rectangular, including hexagonal, pentagonal, octagonal, or other multi-sided polygonal configurations selected to maximize sensing area coverage and minimize dead zones when the substrate is conformed to the curved surface.

The sensor elements may include any number of sensors to sense input across a surface of a structure in which the sensory array is applied. For example, if the sensor array is applied to cover a portion of a ball, the covered portion may have a sensor density such that input provided at one part of the portion can be sensed by at least one of the sensor elements covering the portion. Example sensor density for a spherical baseball, for example, may include about 20 to about 700 sensor elements; about 20 sensor elements to about 30 sensor elements; about 30 sensor elements to about 40 sensor elements; about 40 sensor elements to about 50 sensor elements; about 50 sensor elements to about 60 sensor elements; about 60 sensor elements to about 70 sensor elements; about 70 sensor elements to about 80 sensor elements; about 80 sensor elements to about 90 sensor elements; about 90 sensor elements to about 100 sensor elements; about 100 sensor elements to about 150 sensor elements; about 150 sensor elements to about 200 sensor elements; about 200 sensor elements to about 300 sensor elements; about 300 sensor elements to about 400 sensor elements; about 400 sensor elements to about 500 sensor elements; about 500 sensor elements to about 600 sensor elements; and about 600 sensor elements to about 700 sensor elements. Other structures and/or shapes may vary the number of sensor elements to ensure sensor detection over a surface of the other structure and/or shape.

Described herein is a grip assessment system including a first electronic device and a second electronic device, each including: a thin-film substrate having a hub and a plurality of protrusions each extending from one of a plurality of bridges coupled to the hub, a sensor array including a plurality of sensor elements disposed in a pattern over the plurality of protrusions and interconnected by a plurality of electrical trace lines. Each of the protrusions may be arranged to cover a portion of a curved surface of a shell when deformed about the respective bridges and coupled to the portion of the curved surface such that a distal end of each of the protrusions substantially covers an apex of the shell. The plurality of sensor elements may be divided amongst the plurality of protrusions such that each protrusion comprises a subset of the plurality of sensor elements, with the sensor elements of each protrusion interconnected by electrical trace lines that route signals from each protrusion toward the centralized routing hub or ring bus. In some embodiments, the plurality of electrical trace lines may emanate from each sensor element to an adjacent sensor element or directly to the ring bus, forming a network of interconnected sensing elements that routes signals from the distributed sensor array to the centralized routing hub for multiplexing and signal transmission to the integrated circuit.

Described herein is a conformal sensor device for application or arrangement onto a curved surface having non-zero Gaussian curvature. The sensor device may include at least a thin-film, substantially inelastic substrate having a plurality of discrete regions configured to conformably cover the curved surface. The discrete regions may include patterned cutouts and edge slits positioned to enable localized deformation and substantially prevent warping or buckling when applied to the curved surface. The sensor device may further include a sensor array disposed on the thin-film substrate. The sensor array may include any number of sensor elements arranged in a non-Cartesian pattern and configured to maintain substantially uniform spatial sensing resolution across the curved surface when the substrate is conformed thereto. The sensor device may further include an integrated circuit electrically coupled to the plurality of sensor elements and arranged, programmed, and powered to analyze signals from the sensor array to determine interactions with the curved surface.

Described herein is a grip assessment system for substantially curved surfaces including: an inelastic thin-film substrate structured into a plurality of interconnected petal-shaped segments where each segment may be arranged to conform smoothly to a portion of a non-developable, substantially curved surface. The system may include a sensor array disposed on the thin-film substrate. The sensor array may include any number of sensor elements arranged in a non-rectangular, tessellated pattern. Each sensor element may be positioned to maintain uniform spatial coverage when the thin-film substrate is deformed to fit the substantially curved surface. The system may further include patterned cutouts within the thin-film substrate strategically positioned to locally relieve mechanical strain, reduce distortion, and/or prevent wrinkling during application of the substrate to the curved surface. The system may further include multi-layer electrical routing integrated into the thin-film substrate which may include conductive and dielectric layers arranged in a tree-like topology that follows paths of minimal curvature to minimize trace length while maintaining electrical integrity. The system may further include a centralized routing hub connected to the electrical routing in order to multiplex and transmit signals from the sensor array to embedded electronics for processing. In some embodiments, the grip assessment system measures and analyzes grip characteristics, including grip pressure and finger placement on the substantially curved surface, and communicates these analyzed characteristics to an external device.

Described herein are systems and methods for manufacturing a printed sensor array on an inelastic substrate that may substantially conform to a non-developable shape. A method of manufacturing a thin film sensor includes providing a flexible substrate film, depositing conductive electrical trace lines including silver ink on the flexible substrate film, applying a dielectric insulating ink at predetermined locations to form bridges on the conductive electrical trace lines, and depositing a sensor element including a resistive or pressure sensitive ink on the flexible substrate film. The sensor element may be positioned to overlap with and electrically connect to portions of the conductive electrical trace lines, and the dielectric insulating ink bridges may allow the conductive electrical trace lines to overlap without causing an electrical short.

The manufacturing method may include precise deposition steps on a flexible substrate film. A polymeric substrate such as polyethylene terephthalate (PET), polyethylene naphthalate (PEN), or polyimide (PI) with thickness ranging from about 25 microns to about 125 microns is prepared by cleaning with isopropyl alcohol. Conductive electrical trace lines are screen-printed using silver-based conductive ink (e.g., DuPont 5025 with about 80% silver content) with line widths of about 100 microns to about 250 microns and thickness of approximately 8 microns to about 15 microns, followed by thermal curing at about 120° C for about 30 minutes. Dielectric insulating bridges are positioned using UV-curable polyurethane-acrylate ink (e.g., Electrodag 452SS) at junctions where traces would otherwise create shorts, with bridge dimensions of about 1.5 millimeters × 1.0 millimeters and thickness of about 20 microns to about 30 microns, followed by UV curing at 385 nm for about 60 seconds. The resistive or pressure-sensitive sensing element is screen-printed using carbon-loaded polymer ink (e.g., Electrodag PF-407C with resistance range of 500Ω to 10kΩ) in a substantially rectangular pattern of about 8 millimeters × about 5 millimeters with thickness of about 12 microns to about 20 microns. The method includes curing at about 130° C for about 45 minutes to achieve pressure sensitivity performance of about 0.5 to about 5% resistance change per 100 grams of applied force.

Conventional thin-film electronics manufacturing processes use substrates that maintain precise shape during processing. When the surface to receive a thin film device is a non-developable surface (surfaces having double curvature or non-zero Gaussian curvature that cannot be flattened without distortion), the thin film may provide flexibility to curve around the surface. However, conventional thin films are not manufactured to withstand stretch and deformity utilized for non-developable surfaces including spheres, ellipsoids, and hyperbolic paraboloids.

The systems and methods described herein provide thin film substrates that conform to surfaces having non-zero Gaussian curvature. The substrates provide symmetry with foils in a plane before folding, with petals connecting to a common ring bus to minimize connectors to a circuit board. The petals may be printed with rectangular or non-rectangular discrete electrical elements that maintain constant area overlap under small lateral shifts. A hub-and-petal (or hub-and-spoke) design includes multiple petals (about 8 petals to about 16 petals) extending outward from a central hub, covering portions of curved surfaces by following meridian lines. Each petal contains electrical trace lines that enter the interior through positioned slits, connecting to a central hub that handles multiplexing and signal routing. The design employs force sensitive resistor technology with two foil halves joined by an intermediate adhesive layer that prevents pixel separation when conforming to curved surfaces. The petals may be physically connected or implemented as separate floating patches that connect electrically to a main signal bus, with deliberate overprovisioning ensuring complete surface coverage while eliminating blind spots.

A sensor matrix employs triangular, hexagonal, Voronoi, or fractal-like sensor pixels instead of conventional square arrangements, enabling uniform coverage of curved surfaces while reducing routing lines. The tessellated pattern maximizes active sensing area while minimizing dead zones, allowing asymmetric layouts that provide increased sensor resolution in specific regions where greater sensitivity is indicated.

An auxetic metamaterial sensor array transforms a rigid, non-elastic substrate into a flexible sensor system using hierarchical cut patterns that create controlled deformation points throughout the material. Precisely engineered holes, slits, and patterned cuts enable local strain deformation while maintaining structural integrity of sensing elements. This design minimizes out-of-plane buckling, ensuring the sensor array lies flush against curved surfaces. Additional structural reinforcement layers may be printed at high-stress areas such as sharp corners and hinge locations.

In some embodiments, the sensor arrays described herein may be formed at least partially of a thermo-shrink material that utilizes a substrate with controlled thermal shrinkage properties that can be precisely activated after initial application to a curved surface. Heating elements may be strategically applied to cause controlled substrate deformation, with heat application targeted to non-sensing areas to preserve sensor functionality. This thermal treatment allows the sensor array to conform to highly curved surfaces, eliminating residual wrinkles or gaps and providing an additional adjustment mechanism for improved surface adaptation.

In some embodiments, at least a portion of the thin-film substrate of the conformal sensor device may include or be composed of a heat shrink material. The heat shrink material may be selectively activated through localized heat application after the thin-film substrate has been initially applied to the surface having non-zero Gaussian curvature. Upon activation, the heat shrink material may undergo controlled dimensional reduction in targeted regions of the thin-film substrate, causing the substrate to conform more closely to the curved surface and substantially eliminating residual wrinkling or buckling that may remain after initial application. The heat application may be targeted to non-sensing areas of the thin-film substrate to preserve the functional integrity of the sensor elements and electrical trace lines during the shrinkage process. In some embodiments, the heat shrink material may be incorporated into the thin-film substrate without causing activation prior to or during the conforming process, providing enhanced mechanical compliance and conformability to the curved surface through the inherent material properties of the heat shrink substrate.

A method for manufacturing a sensor array for conforming to surfaces having non-zero Gaussian curvature may include cutting foils, gluing, and assembling multi-layers of foil sandwiching an adhesive layer. The film includes: tabs/wings to adhere edges of petals; overlapping petals with active sensing areas and non-active structural areas; strategic cuts/slits for local strain relief; routing of electrical trace lines along meridians; elements in non-Cartesian arrangements; and electronics providing specific physical shapes and functionality. The sensor array may include a polymer core with physical properties similar to those of sports equipment and may be formed from hemispheres with cavities for electronics coupled to circuit boards providing embedded charging coils, batteries, and communication electronics.

The embodiments described herein address technical challenges of applying thin-film sensor arrays to surfaces with non-zero Gaussian curvature. Traditional sensor designs relying on Cartesian layouts and planar substrates are ill-suited for complex curved geometries, leading to uneven sensing coverage, mechanical strain, and signal disruption. Substrate deformation during installation often results in buckling, tearing, or misalignment of printed circuit paths, compromising sensor functionality.

Sensor devices include thin-film substrates engineered to conform to complex curves through discrete petal-shaped regions that deform predictably to minimize strain and distortion. Petal-shaped regions feature patterned cutouts, wave-shaped or sawtooth-shaped edges, and feathered edge slits to enable localized deformation and strain relief. The substrate incorporates auxetic properties with hinge-like regions that locally deform under stress and thermo-shrink materials that undergo controlled shrinkage upon heat application.

In general, routing lines are implemented using multiple layers of conductive and insulating materials following hierarchical tree-like structures with central trunks positioned along paths of minimal curvature and branch lines extending to individual sensing elements. Vertical electrical connections are established using through-hole vias formed by laser ablation or mechanical punching and filled with conductive material. Routing lines from multiple petal regions converge at a centralized hub or ring bus handling multiplexing and communication.

An adhesive layer positioned beneath the bottom foil layer may securely bond sensor petals to curved surfaces, extending through pre-cut openings to align top and bottom foil layers precisely. The adhesive provides structural reinforcement, increases tear resistance, and ensures proper alignment between sensing elements in multiple layers.

The sensor devices described herein may measure grip characteristics including grip pressure, finger placement, and finger orientation, distinguish individual digits and analyze grip mechanics, movement patterns, and interactions over time. For example, the sensor devices described herein may measure input to an outer interactable (e.g., manipulable) surface of a ball, handle, or other equipment in which the sensor devices are installed within or on. The sensors may measure information about the interaction/input. The sensor array conforms to spherical or elliptical surfaces enabling uniform sensing coverage. The integrated circuit includes a processor and memory with instructions to analyze signals and trigger communication about analyzed interactions.

Areas of substrate not printed with sensing elements or wiring lines may be removed using laser cutting or die cutting to reduce surface buckling. For spherical applications, the sensor system includes hemispherical shells enclosing central cavities housing embedded electronics arranged on circular printed circuit boards with radially positioned connectors.

The sensor devices described herein may be used for and/or to mimic the feeling of standard sports equipment (i.e., sports equipment that may be used without sensors) without a user being able to tell the difference, wearable electronics, robotics, medical devices, and automotive systems, supporting multiple sensing modalities including pressure, temperature, humidity, touch, presence, damage detection, and radiation sensing. The system includes wireless communication capabilities and wireless charging features. The techniques provide enhanced adaptability to complex geometries, minimize mechanical stress during application, enable controlled post-application adjustments, facilitate efficient signal transmission, and support diverse sensing modalities across various industries.

In some embodiments, the systems described herein incorporate advanced signal processing capabilities for curved surface sensor applications. The signal processing architecture may include adaptive filtering algorithms that compensate for the mechanical strain effects on sensor signals when the thin-film substrate is conformed to complex geometries. These processing techniques account for the signal variations that may occur due to substrate deformation and provide corrected output signals that accurately represent the applied stimuli regardless of the local curvature of the sensing surface.

The signal processing system may incorporate machine learning algorithms trained on datasets of grip interactions collected from curved sensor arrays, enabling the system to distinguish between different types of surface interactions, classify grip patterns, and predict user behavior based on temporal sensing patterns. The processing capabilities may include real-time analysis of multi-modal sensor data, combining pressure, temperature, humidity, and capacitive measurements to provide comprehensive interaction characterization that exceeds the capabilities of single-modality sensing systems.

In some embodiments, the sensor arrays described herein extend beyond pressure sensing to incorporate comprehensive multi-modal impedimetric sensing capabilities including temperature measurement, wetness detection, humidity sensing, conductive touch detection, presence switches, damage assessment through conductive line monitoring, and photo/radiation detection. These diverse sensing modalities utilize the same thin-film substrate and conformal application techniques while providing expanded characterization capabilities for complex interaction analysis.

The temperature sensing capabilities enable thermal mapping across curved surfaces, providing insights into heat distribution patterns during grip interactions or environmental thermal effects. Wetness and humidity sensors provide environmental monitoring capabilities that may be valuable in sports applications where moisture effects on grip performance are impactful factors. Conductive touch sensors enable capacitive detection of approach and contact events, while presence switches provide binary detection of contact or proximity states.

The sensor devices described herein may include photo/radiation detection capabilities enabling UV exposure monitoring for outdoor sports equipment, radiation detection for safety applications, or optical sensing for proximity detection. These expanded sensing modalities demonstrate the versatility of the conformal sensor platform for diverse application requirements while maintaining the curved surface conformity and uniform spatial resolution characteristics.

In some embodiments, the sensory arrays described herein may be used in sports equipment performance analysis, wearable electronics for health monitoring, soft robotics tactile feedback systems, medical device pressure monitoring, and automotive safety systems. Each application area may incorporate specialized sensor configurations, signal processing algorithms, and mechanical designs optimized for the specific operational requirements and environmental conditions. Sports equipment applications may emphasize high-frequency dynamic response for impact detection and technique analysis, while medical device applications prioritize long-term stability and biocompatibility. The modular design architecture enables customization of sensor density, processing capabilities, and communication interfaces to match specific application requirements while maintaining the curved surface conformity capabilities to, for example, mimic the same feel and weight of sports equipment such as a ball, a handle, or the like.

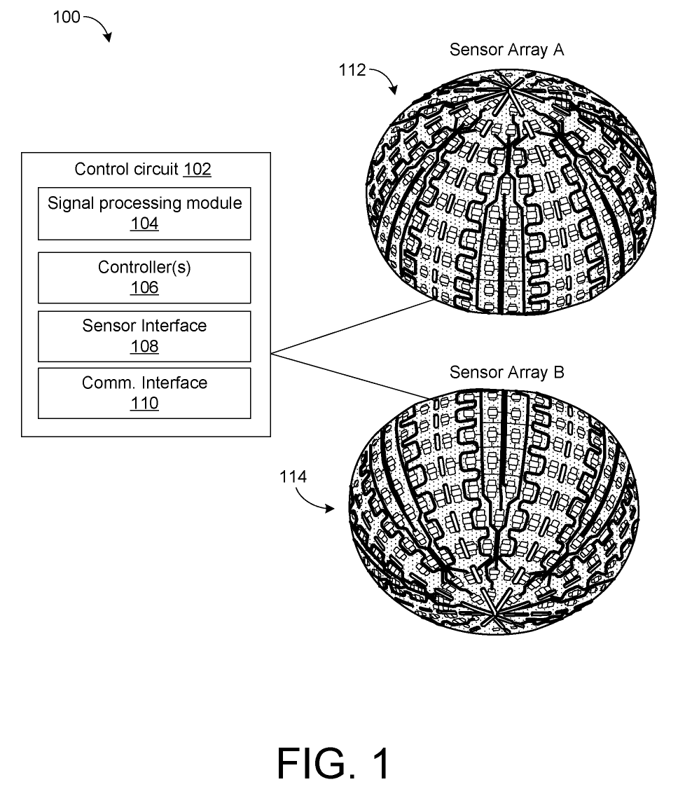

FIG. 1 illustrates a sensor system 100 of a thin film sensor array for grip assessment on curved surfaces in accordance with various aspects of the present disclosure. As depicted in FIG. 1, the sensor system 100 may include one or more of a control circuit 102, a signal processing module 104, controllers 106, a sensor interface 108, and a communication interface 110. The system 100 may include a first portion including a sensor array A 112 and a second portion including a sensor array B 114. The first portion (e.g., sensor array A 112) may mechanically and communicatively couple to the second portion (e.g., sensor array B 114).

The control circuit 102 may include electronic components configured to manage operations within the sensor system 100. The control circuit 102 may include a processor and memory that may store computer-readable instructions for managing signal flow and operational tasks. The control circuit 102 may interact with other components, such as the signal processing module 104, to coordinate data analysis and communication. In some embodiments, the control circuit 102 may include alternative configurations, such as distributed control systems or modular control units.

The signal processing module 104 may represent circuitry designed to analyze signals received from sensors within the sensor arrays 112, 114. The signal processing module 104 may include algorithms that may determine interactions with surfaces based on input from sensor arrays. The signal processing module 104 may communicate with the control circuit 102 to relay processed data for further actions. In some embodiments, the signal processing module 104 may include additional features, such as adaptive filtering or machine learning-based signal interpretation.

The controllers 106 may regulate and coordinate the functions of various components in the sensor system 100. The controllers 106 may include hardware or software elements that may manage the timing and synchronization of operations across the system. The controllers 106 may interact with the sensor interface 108 to ensure communication between sensor arrays and other modules. In some embodiments, the controllers 106 may include programmable logic controllers or microcontroller-based systems for processing sensor measurements from the sensor elements to determine grip characteristics including grip pressure, finger placement, and finger orientation on curved surfaces having non-zero Gaussian curvature. The controllers 106 may analyze interaction data to distinguish individual digits of a user gripping the surface and identify grip mechanics, movement patterns, and interactions over time periods. The controllers 106 may also manage multi-modal sensing including measurements of pressure, vibration, shock, acceleration, stress, force, touch, humidity, presence, damage, radiation, and temperature from the sensor array disposed in non-Cartesian patterns. The controllers 106 may control signal routing through the tree-like electrical interconnection structure and centralized hub or ring bus to multiplex signals from multiple petal-shaped segments of the thin-film substrate. The controllers 106 may execute real-time processing of signals from sensor elements conforming to surfaces with double curvature to provide substantially uniform sensing coverage. The controllers 106 may coordinate wireless communication to transmit analyzed grip characteristics and interaction data to external devices for sports equipment analysis, wearable electronics, robotics, medical devices, or automotive applications. The controllers 106 may regulate power management including wireless charging functions and battery optimization for embedded electronics housed within hemispherical shells or curved sensor housings of either or both sensor array A 112 and sensor array B 114.

The sensor interface 108 may include connections that facilitate communication between the sensor arrays and other modules. The sensor interface 108 may include electrical connectors or wireless communication elements that may transmit data from sensor arrays to the signal processing module 104. The sensor interface 108 may interact with the communication interface 110 to enable external data exchange. In some embodiments, the sensor interface 108 may include alternative designs, such as optical or magnetic coupling mechanisms.

The communication interface 110 may represent hardware or software elements that enable data exchange with external devices. The communication interface 110 may include wireless communication protocols, such as Bluetooth or Wi-Fi or other wireless technology, to transmit processed data to external systems. The communication interface 110 may interact with the control circuit 102 to manage outgoing and incoming data streams. In some embodiments, the communication interface 110 may additionally include wired communication options, such as USB or Ethernet connections. In some embodiments, the wireless communication capabilities of the conformal sensor devices described herein may include one or more of Bluetooth, Bluetooth Low Energy, Wi-Fi, near field communication, Zigbee, or other short-range or long-range wireless communication protocols. The wireless communication module may be integrated into the printed circuit board housed within the interior cavity of the hemispherical shell and may be configured to transmit analyzed grip characteristics, raw sensor data, device status information, and power management data to external devices including smartphones, tablets, laptop computers, dedicated monitoring systems, or cloud-based data processing platforms. In some embodiments, the wireless communication module may support bidirectional communication, enabling external devices to transmit configuration parameters, sensitivity thresholds, calibration data, and firmware updates to the integrated circuit of the conformal sensor device

The sensor array A 112 may include a collection of sensor elements arranged to detect interactions on curved surfaces, as described elsewhere herein. The sensor array A 112 may include sensors that may measure parameters such as pressure, impedance, temperature, or capacitive touch. The sensor array A 112 may include sensor elements that may conform to surfaces with non-zero Gaussian curvature. The sensor array A 112 may interact with the sensor interface 108 to relay raw data for processing. In some embodiments, the sensor array A 112 may include alternative configurations, such as tessellated or fractal-like sensor arrangements. In some embodiments, the sensors may be distributed across petal-shaped regions of an inelastic substrate. These regions may be deformed predictably when applied to a curved form, enabling uniform spatial sensing coverage with minimal wrinkling or distortion.

The sensor array B 114 may provide additional sensing capabilities to complement the functionality of sensor array A 112. The sensor array B 114 may include a collection of sensor elements arranged to detect interactions on curved surfaces, as described elsewhere herein. The sensor array B 114 may include sensors that may measure parameters such as pressure, impedance, temperature, or capacitive touch. The sensor array B 114 may include sensor elements that may conform to surfaces with non-zero Gaussian curvature. The sensor array B 114 may interact with the sensor interface 108 to relay raw data for processing. In some embodiments, the sensor array B 114 may include alternative configurations, such as tessellated or fractal-like sensor arrangements. In some embodiments, the sensors may be distributed across petal-shaped regions of an inelastic substrate. These regions may be deformed predictably when applied to a curved form, enabling uniform spatial sensing coverage with minimal wrinkling or distortion. The wrinkling and/or distortion may increase noise in the sensor data and/or reduce effectiveness of at least some of the sensors in the sensor array.

In some embodiments, sensor array A 112 may be positioned on a first portion (e.g., one quarter, one third, one half, etc.) of a curved surface opposite the sensor array B 114 positioned on a second portion (e.g., one quarter, one third, one half, etc.) of the curved surface. In some embodiments, both sensor array A 112 and sensor array B 114 may be electrically coupled to the control circuit 102 through conductive routing lines. In some embodiments, the sensor array A 112 and sensor array B 114 may be electrically coupled to one another through any number of conductive routing lines. In some embodiments, the sensor array A 112 and sensor array B 114 may function as standalone units and may not be electrically coupled; instead, each sensor array 112, 114 may be coupled to a separate control circuit 102.

In general, the sensor array A 112 and sensor array B 114 may include tessellated sensing elements arranged in non-Cartesian layouts, which may conform to the curvature of the surfaces. The control circuit 102 may manage the hierarchical routing architecture, with trunk lines extending along geodesic paths and branch lines addressing individual sensing elements. Through-foil vias may establish vertical electrical connections between conductive layers, allowing the sensor arrays A 112 and B 114 to operate in multi-layer configurations without increasing the lateral footprint.

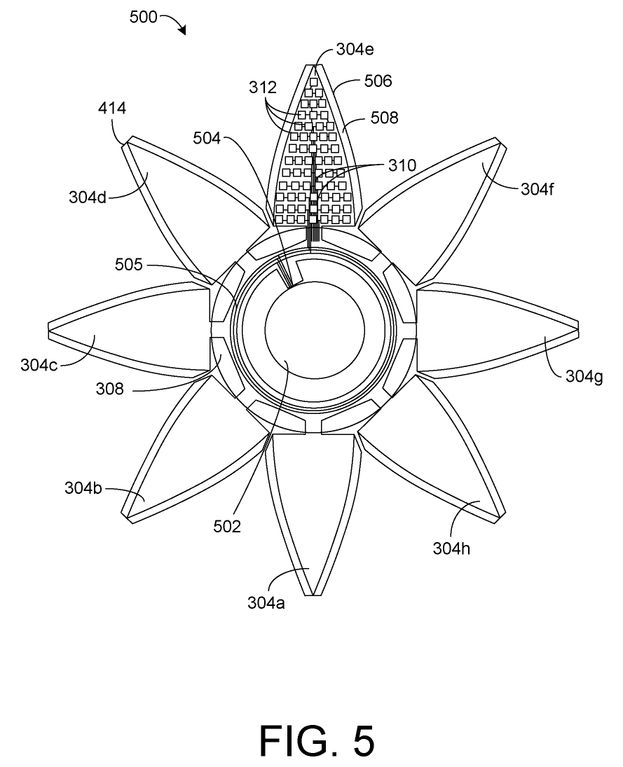

As shown in FIG. 1, the sensor array A 112 and sensor array B 114 may each be depicted as a three-dimensional hemispherical object having a patterned surface. The patterned surface may include a number of radial strip-like features representing sensors arranged in longitudinal bands that follow the curvature of each respective hemispherical surface. The repeating geometric shapes visible on the surface of each of the sensor arrays A 112 and sensor B 114 may correspond to individual sensor elements distributed across the petal-shaped regions of the thin-film substrate, as described in greater detail with respect to FIG. 5 below.

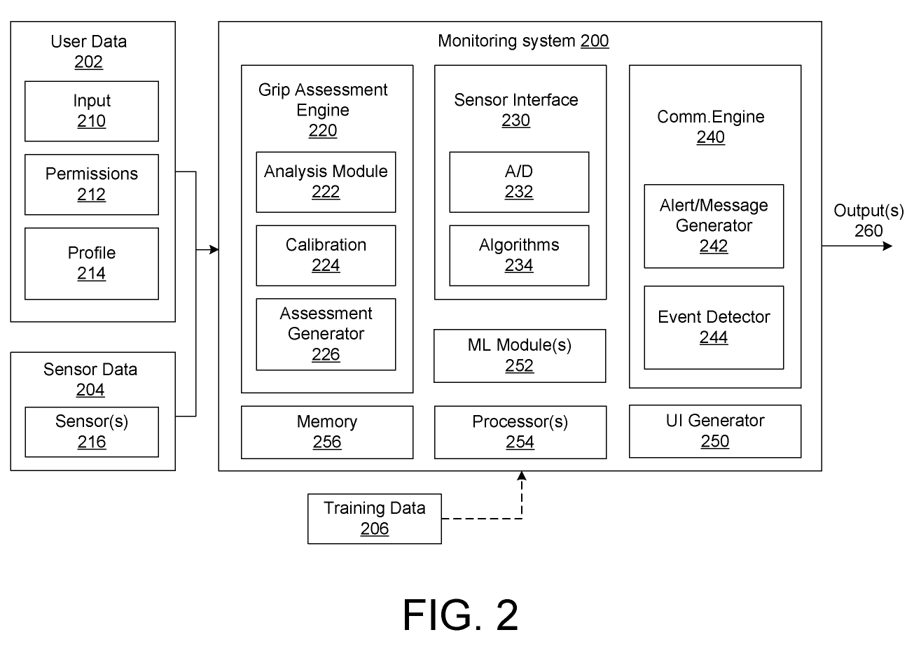

FIG. 2 illustrates a monitoring system 200 for a thin film sensor array for monitoring and assessing a grip on curved surfaces in accordance with various aspects of the present disclosure. The monitoring system 200 may utilize received user data 202, detected or received sensor data 204, and/or received training data 206. The user data 202 may include user input 210, user permissions 212, and/or user profile data 214. The user input 210 may include sensor settings, ball settings, pitch settings, or monitoring requests or settings associated with operation and use of device 100 and/or operation of control circuit 102. The user permissions 212 may include predefined permission rules or permissions set by a manufacturer or a user for accessing, handling, and using user input 210 and/or user profile data 214. The user profile data 214 may include grip interaction profiles, finger placement patterns, pressure distribution measurements, and/or temporal grip sequences collected from users interacting with one or more sensors on device 100. In some embodiments, the user profile data may store calibration parameters for individual user grip characteristics, including finger span measurements, grip strength baselines, and/or preferred interaction zones on spherical or elliptical surfaces. In some embodiments, the user profile data 214 may include user-specific configurations including hand geometry parameters, grip preferences, and performance metrics derived from interactions with the sensor device 100. In some embodiments, the profile data 214 may further include demographic information, skill level assessments for sports applications, and/or customized sensitivity thresholds for the plurality of sensor elements disposed on the device 100.

The sensor data 204 may represent data captured by one or more sensor(s) 216 on sensor array 112 and/or sensor array 114. In some embodiments, the sensor data 204 represents a portion of the data captured by the sensor(s) 216 over a period of time. In some embodiments, the sensor data 204 represents all data captured by the sensors 216 over a period of time. The sensors 216, may include one or more sensor elements arranged on a portion of a ball or other object.

The sensor(s) 216 may include devices capable of detecting physical or environmental parameters. The sensors 216 may be standalone or arranged in one or more sensor arrays. In some embodiments, the sensors 216 may measure pressure, vibration, shock, acceleration, stress, force, touch, humidity, presence, damage, radiation, and/or temperature. In some embodiments, the sensor(s) 216 may include impedimetric sensor elements in tessellated patterns including non-square shapes such as triangular, hexagonal, rectangular, or polygonal shapes that maintain substantially uniform sensing coverage and ability across surfaces with double curvature. The sensor(s) 216 may transmit raw data to a sensor interface (e.g., sensor interface 230) for further processing. The sensor(s) 216 may interact with the grip assessment engine 220 to determine grip-related metrics.

The monitoring system 200 may include a grip assessment engine 220, a sensor interface 230, and a communication engine 240. The grip assessment engine 220 may analyze input data to determine grip-related metrics including, but not limited to grip pressure distribution, finger placement accuracy, and grip stability metrics. The grip assessment engine 220 may include algorithms designed to interpret sensor data from the sensor(s) 216. The grip assessment engine 220 may process data to determine metrics such as grip pressure, finger placement, and/or movement patterns. The grip assessment engine 220 may include an analysis module 222 to refine grip-related insights. In some embodiments, the grip assessment engine 220 may include calibration routines 224 for particular user profiles. In some embodiments, the grip assessment engine 220 may process data from the sensor arrays to identify individual digits, measure grip force vectors, and assess grip technique quality for sports equipment applications.

The analysis module 222 may perform real-time signal processing of data from the centralized routing hub or ring bus described elsewhere herein, applying algorithms to distinguish between different grip patterns, detect grip anomalies, and calculate performance metrics. The analysis module 222 may correlate sensor measurements with user profiles 214 to provide personalized feedback and trend analysis through a grip assessment generator 226. In some embodiments, the analysis module 222 may include computational routines for interpreting raw or processed data. The analysis module 222 may interact with the grip assessment engine 220 to refine grip-related metrics. The analysis module 222 may transmit processed data to a user interface (UI) generator 250 for output 260 of grip graphics, statistics, grip assessments, coaching tips, diagrams, or the like. In some embodiments, the analysis module 222 may include visualization tools for presenting data trends using the UI generator 250.

The sensor interface 230 may provide electrical and communication connectivity between the sensor arrays, sensors 216, and processing modules of FIGS. 1 and 2. In some embodiments, the sensor interface 230 may be the same as or similar to the sensor interface 108, as described herein. The sensor interface 230 may include signal conditioning circuits to process multiplexed data from the centralized routing hub or ring bus, as described elsewhere herein, accounting for signal variations caused by substrate deformation when conforming to the surfaces described herein. The sensor interface 230 may manage multi-layer electrical routing signals transmitted through the tree-like interconnection structure, including data from through-hole vias that establish vertical electrical connections between non-adjacent conductive layers. The sensor interface 230 may include analog-to-digital (A/D) converters 232 calibrated for sensors 216 measuring at least one of pressure, vibration, shock, acceleration, stress, force, touch, humidity, presence, damage, radiation, and temperature across tessellated sensor patterns. The sensor interface 230 may coordinate wireless communication protocols for transmitting sensor data from embedded electronics housed within the sensor arrays (e.g., sensor arrays 112, 114) to external processing systems. The sensor interface 230 may include compensation algorithms 234 to maintain signal integrity despite mechanical strain relief from patterned cutouts, wave-shaped edges, and auxetic structures in the thin-film substrate. The sensor interface 230 may synchronize data collection timing across multiple sensors 216 distributed over curved geometries to ensure substantially uniform sensing coverage and accurate grip assessment analysis.

The communication engine 240 may facilitate data exchange between system 200 components. The communication engine 240 may include protocols for transmitting data between the sensor interface 230 and the grip assessment engine 220 and/or external computing devices (not shown). The communication engine 240 may include an alert/message generator 242 to transmit notifications about sensors, grip assessments, users, or other system 200 analysis. The communication engine 240 may include encryption routines for secure data transmission. In some embodiments, the communication engine 240 may include wireless communication modules for wirelessly communicating with external computing devices.

The alert/message generator 242 may produce notifications or messages based on detected events detected by event detector 244. For example, the alert/message generator 242 may generate and send notifications based on grip assessment results generated by grip assessment engine 220 from sensor data 204. The grip assessment results may include, but are not limited to technique improvement suggestions, equipment maintenance alerts, safety warnings for excessive grip pressure, and/or performance achievement notifications. The alert/message generator 242 may format messages for transmission through the wireless communication capabilities of the system 200. In some embodiments, the alert/message generator 242 may include routines for formatting alerts into user-readable formats. The alert/message generator 242 may interact with the event detector 244 to determine when notifications can be triggered for sending. The alert/message generator 242 may include options for customizing notification formats. In some embodiments, the alert/message generator 242 may include priority settings for different types of alerts.

The event detector 244 may identify grip events including, but not limited to, grip initiation, grip release, grip adjustments, and/or unusual pressure patterns that may indicate equipment malfunction, user fatigue, or technique errors. The event detector 244 may monitor the wireless communication signals from embedded electronics (e.g., sensor array A 112 and sensor array B 114) housed within a ball, for example, to detect system status changes.

The system 200 may further include or access machine learning (ML) model(s) 252. The ML models 252 may include algorithms trained to interpret data patterns in the sensor data 204, for example. The ML model(s) 252 may include supervised or unsupervised learning techniques. The ML model(s) 252 may interact with the training data 206 to refine predictive accuracy. The ML model(s) 252 may transmit insights to the analysis module 222 for further processing. In some embodiments, the ML model(s) 252 may include pre-trained models for specific applications for grip assessment and grip coaching. The training data 206 may include datasets of grip interactions collected from the sensor arrays, including labeled grip pressure patterns, finger placement coordinates from petal-shaped segments, and temporal sequences of grip adjustments. The training data 206 may include data from sports equipment analysis, wearable electronics applications, and medical device monitoring to train the ML model(s) 252 for diverse use cases. In some embodiments, the ML models 252 may be trained on grip assessment data to predict user performance, classify grip types, recognize individual users based on grip signatures, and/or optimize sensor sensitivity for different applications. The ML model(s) 252 may process training data 206 from multiple users interacting with the sensor device 100 across various curved geometries.

The system 200 may include one or more processor(s) 254 that may execute instructions to perform computational tasks associated with system 200 and device 100. The processor(s) 254 may include multi-core architectures for parallel processing. The processor(s) 254 may interact with the memory 256 to retrieve stored instructions. The processor(s) 254 may transmit results to the UI generator 250 for user engagement. In some embodiments, the processor(s) 254 may include specialized hardware for machine learning tasks.

The system 200 may include memory 256 to store data and instructions for system 200 operations. The memory 256 may include volatile or non-volatile storage components. The memory 256 may interact with the processor(s) 254 to execute stored instructions. The memory 256 may store datasets for the training data 206. In some embodiments, the memory 256 may include hierarchical storage for organizing data.

The memory 256 can include one or more non-transitory computer-readable storage media. The memory 256 may store instructions and data that are usable in combination with processors 254 to execute the processes and/or algorithms described herein as well as to execute or interface with grip assessment engine 220, sensor interface 230, communication engine 240, and UI generator 250. The memory 256 may also function to store or have access to the ML models 252.

The UI generator 250 may generate alerts and/or interactive elements for user engagement. The user interface generator 250 may include routines for designing graphical or textual interfaces. The user interface generator 250 may interact with the communication engine 240 to transmit user inputs. The user interface generator 250 may include templates for different device types. In some embodiments, the user interface generator 250 may include accessibility features for diverse user needs.

The training data 206 may include datasets used to refine the ML model(s) 252. The training data 206 may include labeled or unlabeled data samples for grip mechanics. The training data 206 may interact with the analysis module 222 to determine data patterns in grip analysis. The training data 206 may include subsets for specific applications including, but not limited to imaging analysis for reviewing grip data, sports performance analysis apps for technique optimization in baseball, tennis, golf, football, and other grip-dependent sports, rehabilitation apps for monitoring patient progress in physical therapy and hand strength recovery, gaming and virtual reality apps for gesture recognition and haptic feedback calibration, ergonomic assessment apps for workplace safety and tool design optimization, biometric security apps for user identification based on unique grip signatures, automotive safety apps for monitoring driver attention and grip patterns on steering wheels, prosthetic control apps for training artificial limbs to respond to grip intention signals, musical instrument training apps for analyzing finger placement and pressure in piano, guitar, or violin instruction, industrial quality control apps for monitoring worker grip consistency in manufacturing processes, accessibility apps for adaptive device control based on individual grip capabilities, robotic training apps for teaching machines proper object manipulation and grip force control, medical diagnostic apps for detecting neurological conditions through grip pattern analysis, and fitness tracking apps for monitoring exercise form and grip strength progression. In short, the training data 206 may be used by any number of applications and may be collected from the sensor arrays with their ability to measure grip pressure, finger placement, and temporal grip patterns across curved surfaces having non-zero Gaussian curvature.

The output(s) 260 may represent data transmitted from the UI generator 250 to an external display or companion device. The output(s) 260 may include grip graphics, statistics, grip assessments, technique adjustments, coaching tips, and diagrams generated by the UI generator 250 based on processed data from the analysis module 222 and the grip assessment generator 226.

The grip assessment engine 220 may include the analysis module 222, the calibration routines 224, and the grip assessment generator 226. The analysis module 222 may receive processed data from the sensor interface 230 and may interact with the calibration routines 224 to apply user-specific calibration parameters before transmitting refined grip metrics to the grip assessment generator 226.

In some embodiments, the conformal sensor devices described herein may be characterized as a grip assessment device or grip assessment sensor that can measure grip characteristics of a user interacting with the substantially curved surface. As a grip assessment device, the conformal sensor device may provide comprehensive measurement and analysis of grip pressure distribution, finger placement accuracy, finger orientation, grip stability metrics, and individual digit identification across the substantially curved surface. The grip assessment functionality may be enabled by the combination of a non-cartesian sensor element arrangement providing uniform spatial sensing resolution across the curved surface, the patterned cutouts enabling smooth conformity of the thin-film substrate to the curved surface, and the integrated circuit configured to analyze signals from the sensor array to determine grip-related interactions with the surface.

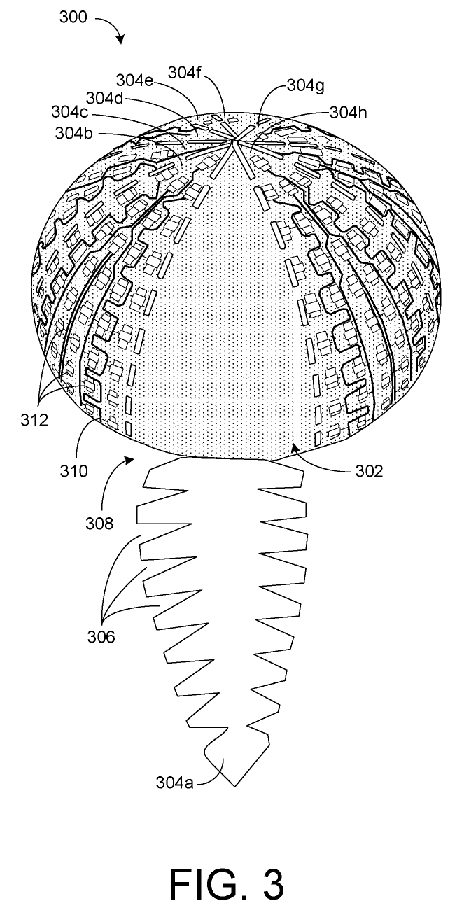

FIG. 3 illustrates a portion 300 of a spherical device 100 including thin film sensor arrays for grip assessment. As depicted in FIG. 3, the portion 300 depicts a spherical shell 302, petal-shaped segments 304 (e.g., segments 304a-304h), patterned cutouts 306, a central hub 308 (see FIG. 5), electrical trace lines 310, sensors 312, and/or other components. The spherical shell 302 may include a curved structure to accommodate arrays of thin film sensors. The spherical shell 302 may be a top or bottom portion of a full hemisphere device coupled together through the central hub 308. Two spherical shells 302 may enclose a central cavity for embedded electronics by coupling through the hub 308. The spherical shell 302 may include routing channels or slits along its equator that may allow internal trace lines to connect external sensor foils to the internal printed circuit board. In some embodiments, the spherical shell 302 may include alternative geometries, such as ellipsoids or hyperbolic paraboloids, prolate spheroids, cylinders, etc. to support applications with non-developable surfaces.

In some embodiments, each protrusion may include a first edge having a first set of cutouts extending along a first side of a periphery of the respective protrusion and a second edge having a second set of cutouts extending along a second side of the periphery of the respective protrusion, with the first and second sets of cutouts positioned to provide symmetric strain relief on opposing sides of the protrusion during deformation about the respective bridge connection.

The petal-shaped segments 304 may represent discrete regions of thin film having sensors 312 thereon. The segments 304a-304h may conform to the spherical shell 302. The petal-shaped segments 304a-304-h may include radial sections that may extend from the equator to the poles of the spherical shell 302. The petal-shaped segments 304a-304h may include active sensor regions and inactive structural regions that may overlap adjacent segments 304 to improve adhesion and alignment. In some embodiments, the petal-shaped segments 304a-304h may include interlocking patches or floating sections that may connect electrically to the central hub 308. In this example, segment 304a is not yet overlaid onto the spherical shell 302 and segments 304b-304h are overlaid onto shell 302 to adhere or otherwise conform or deform to the shape of the outer surface of the spherical shell 302.

The patterned cutouts 306 may include a number of wave-shaped or sawtooth-shaped openings along a perimeter of each of the petal-shaped segments 304a-304h. The openings or cutouts support localized deformation of the petal-shaped segments 304. The patterned cutouts 306 may include auxetic slits or edge feathering that may allow controlled deformation under mechanical stress. The patterned cutouts 306 may include negative spaces that may reduce surface buckling and distribute mechanical strain. In some embodiments, the patterned cutouts 306 may include hierarchical cut patterns or mesh-like designs to support complex geometries.

In some embodiments, at least a portion of the plurality of patterned cutouts may be positioned along a perimeter of the thin-film substrate, including along outer edges of the petal-shaped segments and along boundary regions between adjacent petal-shaped segments, to provide strain relief at the outermost regions of the substrate where mechanical stress may be greatest during application to the curved surface.

The central hub 308 may provide a connection point for electrical trace lines 310 within the spherical shell 302. The central hub 308 may include a multiplexing system that may handle signal routing from multiple petal-shaped segments 304a-304h. The central hub 308 may include radial connectors arranged around a perimeter to align with trace lines 310 from the petal-shaped segments 304a-304h. In some embodiments, the central hub 308 may include a ring bus 505 (see FIG. 5) configuration to reduce a number of direct connections to a processor on a circuit board installable within the central hub 308.

The electrical trace lines 310 may include conductive pathways arranged to connect sensor elements 312 to the central hub 308. The electrical trace lines 310 may include tree-like routing architectures that may follow geodesic paths of minimal curvature. The electrical trace lines 310 may include multilayer configurations with conductive and dielectric inks to support intersecting paths. In some embodiments, the electrical trace lines 310 may be arranged substantially along a central meridian of each of the petal-shaped segments and within a predetermined clearance range defined from a perimeter of each respective petal-shaped segment.

The sensor elements 312 may represent impedimetric or other sensorized components arranged in a non-Cartesian pattern on the petal-shaped segments 304a-304h. In some embodiments, the sensor elements 312 may be the same as or similar to the sensor array A 112 and/or sensor array B 114, as described elsewhere herein.

In some embodiments, the spherical shell 302 may serve as a primary surface to which the petal-shaped segments 304a-304h conform. The petal-shaped segments 304a-304h may be arranged in an overlapping configuration along the curved surface of the spherical shell 302, with patterned cutouts 306 positioned within the segments to accommodate mechanical strain during deformation. The central hub 308 may be located at the geometric center of the spherical shell 302, with electrical trace lines 310 extending radially outward from the hub to connect to sensor elements 312 distributed across the petal-shaped segments 304a-304h.

In some embodiments, the patterned cutouts 306 within the petal-shaped segments 304 may include wave-shaped or sawtooth-shaped designs that allow the substrate to deform predictably when applied to the spherical shell 302. The electrical trace lines 310 may follow hierarchical tree-like paths, with trunk lines extending along geodesic curves of minimal curvature and branch lines connecting to individual sensor elements 312. In some embodiments, the sensor elements 312 may be arranged to maintain uniform spatial sensing resolution across the curved surface of the spherical shell 302. In some embodiments, the sensor elements 312 may be arranged in tessellated patterns, such as hexagonal or triangular configurations that maintain the uniform spatial sensing resolution.

In some embodiments, the central hub 308 may act as a multiplexing node, consolidating signals from the sensor elements 312 through the electrical trace lines 310. The hub may include a circular printed circuit board with radially arranged connectors that interface with the trace lines 310 extending from the petal-shaped segments 304a-304h. The adhesive layer beneath the petal-shaped segments 304a-304h may secure the assembly to the spherical shell 302 while aligning the patterned cutouts 306 with the electrical trace lines 310 to maintain structural integrity and signal transmission.

The dome-shaped upper portion of the assembly 300 may correspond to the spherical shell 302 in its assembled state, with segments 304b-304h conformed and adhered to the outer surface of the spherical shell 302. The shaft-like lower portion of the assembly, having a tapered profile, may correspond to the petal-shaped segment 304a in its undeployed state, prior to being folded and conformed to the spherical shell 302.

In some embodiments, petals may converge at the apex of the spherical shell 302, corresponding to the distal ends of the petal-shaped segments 304b-304h meeting at the pole of the spherical shell 302. Each petal-shaped segment 304b-304h may be deformed about its respective bridge region 308 and conformed to the curved surface of the spherical shell 302 such that the distal end of each segment converges at the apex, resulting in overlapping portions of the thin-film substrate that provide substantially uniform sensing coverage at the pole of the spherical shell 302.

The sawtooth-like or jagged outer edges visible along the undeployed petal-shaped segment 304a may correspond to the patterned cutouts 306 along the perimeter of the segment. The patterned cutouts 306 may include wave-shaped or sawtooth-shaped openings that enable localized deformation and strain relief when the segment 304a is deformed about the bridge region 308 and conformed to the curved surface of the spherical shell 302. The depth and spacing of the patterned cutouts 306 may be selected based on the diameter or radius of curvature of the spherical shell 302 to minimize sensor performance degradation of one or more of the plurality of sensor elements when the conformal sensor device is curved about the surface.

The region 308 may be visible near the upper portion of the undeployed petal-shaped segment 304a at the junction between the shaft-like lower portion and the dome-shaped upper portion of the assembly 300. The region 308 may correspond to a bridge region about which the petal-shaped segment 304a may be deformed when applied to the spherical shell 302. The bridge region 308 may function as a flexible connection point that allows the petal-shaped segment 304a to bend and conform to the curved surface of the spherical shell 302 while maintaining electrical continuity between the central hub and the sensor elements disposed on the segment 304a. The bridge region 308 may be positioned to minimize mechanical stress concentration during deformation while preserving signal integrity throughout the conformal application process.

Any number of sensor elements 312 and electrical trace lines 310 may be visible along the surface of the undeployed petal-shaped segment 304a and along the conformed segments 304b-304h on the dome portion of the assembly. The sensor elements 312 and electrical trace lines 310 may correspond to the printed sensor array and conductive routing paths disposed on the surface of the petal-shaped segment 304a, arranged in a non-Cartesian pattern and routed substantially along the central meridian of the segment toward the central hub 308. The electrical trace lines 310 may be arranged within a predetermined clearance range defined from the perimeter of the petal-shaped segment 304a to minimize mechanical stress on the conductive pathways when the segment 304a is deformed about the bridge region 308 and conformed to the curved surface of the spherical shell 302.

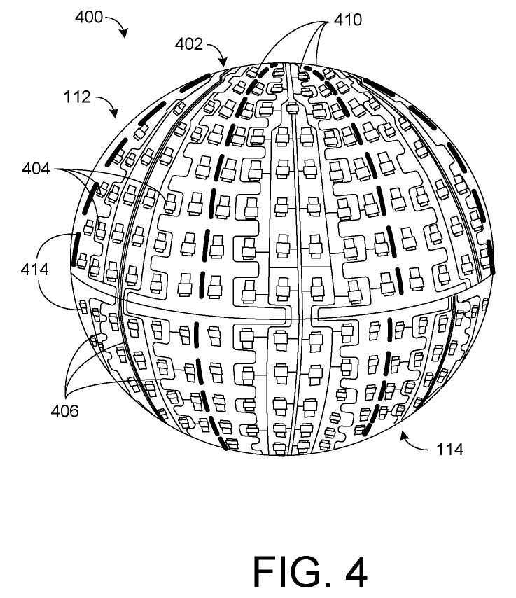

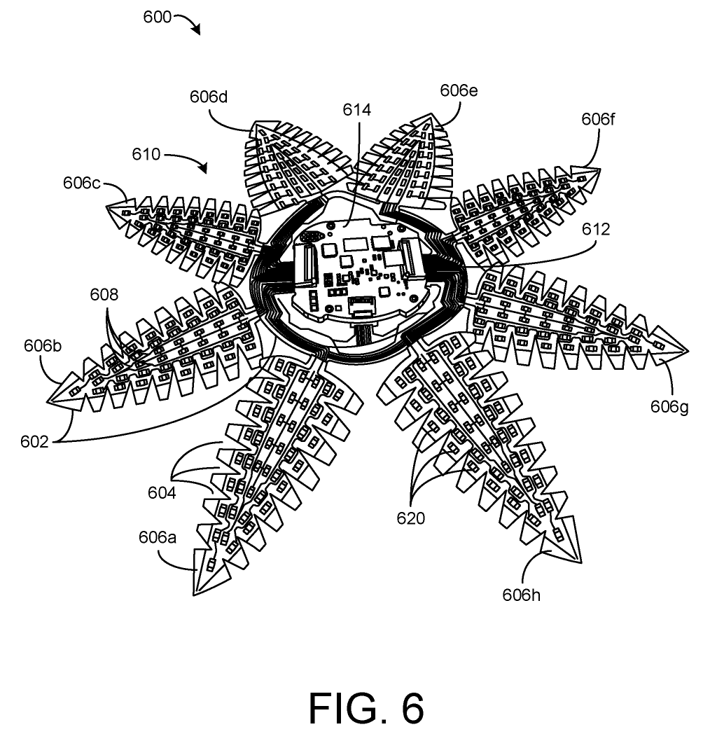

FIG. 4 illustrates a spherical sensor device 400 for performing grip analysis on curved surfaces. The sensor device 400 may include one or more of a sensor array 402 (e.g., a first hemisphere array 112 and a second hemisphere array 114), sensor elements 404, electrical trace lines 406, a ring bus 505 (installed between the two sensor arrays 402 similar to FIG. 5), petal-shaped segments 410, patterned cutouts 412, a thin-film substrate 414, an integrated circuit 416 (see FIG. 6), and/or other components.

The sensor arrays 402 may include a configuration of interconnected sensing elements arranged on a thin-film substrate 414. In some embodiments, the sensor arrays 402 may be the same as or similar to the sensor array A 112, sensor array B 114, and/or sensor interface 108, as described elsewhere herein. The sensor elements 404 may represent individual sensors arranged in a non-Cartesian pattern about each of the sensor arrays 402. In some embodiments, the sensor elements 404 may be the same as or similar to the sensor elements 312, as described elsewhere herein. While three sensor elements are labeled, the similarly shaped structures along the surfaces of sensor arrays 402 depict a number of additional sensor elements 404. Each sensor element 404 may include or represent one or more sensor types. In some embodiments, sensor elements 404 are different sensors grouped along the surfaces of sensor arrays 402. In some embodiments, the sensor elements 404 are electrically interconnected. In some embodiments, particular rows, columns, or areas of sensor elements 404 are electrically interconnected while other groups are electronically interconnected amongst separate groups of sensor elements 404. In some embodiments, each sensor element 404 or group of sensor elements 404 may be electrically and communicatively coupled to the ring bus 505.

The electrical trace lines 406 may represent any number of conductive pathways that connect one or more of the sensor elements 404 to a centralized routing hub 308. In some embodiments, the electrical trace lines 406 may be the same as or similar to the electrical trace lines 310, as described elsewhere herein. The petal-shaped segments 410 may each be discrete regions of the thin-film substrate described herein designed to conform to curved surfaces of non-zero Gaussian curvature (e.g., a spherical object). In some embodiments, the petal-shaped segments 410 may be the same as or similar to the petal-shaped segments 304, as described herein. The patterned cutouts 412 may include strategically positioned slits or openings to minimize mechanical strain during deformation. In some embodiments, the patterned cutouts 412 may be the same as or similar to the patterned cutouts 306, as described herein.

The thin-film substrate 414 may include or be formed of a flexible but inelastic material that may be appliable to conform to surfaces with non-zero Gaussian curvature. The thin-film substrate 414 may include materials such as polyimide or polyethylene terephthalate that may resist stretching while maintaining flexibility. The thin-film substrate 414 may include multiple layers, some of which may include patterned cutouts 412 to enable adhesion and reduce mechanical stress. In some embodiments, the thin-film substrate 414 may include alternative materials, such as polyethylene naphthalate or thermo-shrink materials.

The integrated circuit 416 (FIG. 6) (e.g., a printed circuit board (PCB)) may include a processor and memory to analyze signals from the sensor arrays 402. In some embodiments, the integrated circuit 416 may be the same as or similar to the processor(s) 254 and memory 256, as described herein.

In some embodiments, the sensor array 402 may include petal-shaped segments 410 arranged on a thin-film substrate 414, with patterned cutouts 412 positioned between the segments to accommodate deformation when applied to curved surfaces. The sensor elements 404 may be distributed across the petal-shaped segments 410 in tessellated patterns, such as hexagonal or triangular configurations, to maintain uniform spatial sensing resolution. Electrical trace lines 406 may extend from the sensor elements 404 along geodesic paths of minimal curvature, converging at a ring bus 505 that may serve as a central routing hub for signal transmission.

In some embodiments, the integrated circuit 416 may be electrically coupled to the ring bus 505, which may aggregate signals from the electrical trace lines 406 connected to the sensor elements 404. The patterned cutouts 412 may relieve mechanical strain on the thin-film substrate 414, allowing the petal-shaped segments 410 to conform smoothly to surfaces with non-zero Gaussian curvature. The electrical trace lines 406 may include conductive layers printed with conductive ink and dielectric insulating layers to prevent electrical shorts at crossing points, while through-hole vias may establish vertical connections between non-adjacent conductive layers.

The spherical sensor device 400 may be depicted as a complete, assembled three-dimensional sphere. The sphere may be shown with assembled portions 112 and 114 electrically coupled together to ensure that hemisphere portion 112 operates with hemisphere portion 114. The two hemispheres may be coupled at a central equator location at which the ring bus 505 may be positioned between the two sensor arrays 402, as described in detail with respect to FIG. 5. The sensor elements 404 may be distributed substantially uniformly across the visible surface of the spherical sensor device 400, consistent with the non-cartesian tessellated arrangement described elsewhere herein. The electrical trace lines 406 may be visible as vertically oriented strip regions running longitudinally along the petal-shaped segments 410 on both the upper hemisphere 112 and the lower hemisphere 114, routing signals from the sensor elements 404 toward the ring bus 505.

FIG. 5 illustrates an example thin film sensor array 500. The sensor array 500 may include one or more of a printed circuit board (PCB) 502 coupled to a connector 504, coupling rows of trace lines to a ring bus 505. The array 500 may further include an overlap 506 and a working area 508. The PCB 502 (e.g., integrated circuit 416) may include conductive layers arranged to support electrical connections for the sensor array. The PCB 502 may include alternating conductive and dielectric layers that may be printed onto a thin-film substrate 414. The conductive layers may carry electrical signals between sensor elements 312 and sensor array 402, while dielectric layers may prevent short circuits at crossing points. The PCB 502 may interface with electrical trace lines 310 to route signals from the sensor array 402 to the central hub 308. In some embodiments, the PCB 502 may include through-hole vias to establish vertical signal paths between non-adjacent conductive layers.

The connector 504 may represent an interface for linking row-specific electrical traces within the sensor array 500. The connector 504 may include conductive pathways that may connect individual rows of sensor elements 312 to the ring bus 505. The connector 504 may be positioned along geodesic paths of minimal curvature to reduce mechanical stress during deformation of the thin-film substrate 414. The connector 504 may interact with the central hub 308 to multiplex signals from multiple rows of sensor elements 312. In some embodiments, the connector 504 may include flexible materials to accommodate bending portions of petal-shaped segments 304a-304h.

The ring bus 505 may provide a centralized routing structure for multiplexing signals from the electrical trace lines 310. The ring bus 505 may include a circular configuration that may connect multiple electrical trace lines 310 to a central hub 308. The ring bus 505 may be positioned within the thin-film substrate 414 to enable signal transmission from the sensor array 500 to the integrated circuit/PCB 416. In some embodiments, the ring bus 505 may include alternative configurations, such as a linear or branched structure, depending on the design of the conformal sensor device.

The overlap 506 may provide a structural configuration that accommodates alignment between adjacent sensor elements 312, for example. The overlap 506 may include regions of the thin-film substrate 414 that may extend beyond the perimeter of petal-shaped segments 304a-304h. The overlap 506 may ensure consistent spatial coverage of sensor elements 312 across surfaces with non-zero Gaussian curvature. The overlap 506 may interact with adhesive layers positioned below the thin-film substrate 414 to maintain alignment during application to curved surfaces. In some embodiments, the overlap 506 may include patterned cutouts 306 to minimize buckling deformation of the thin-film substrate and to minimize signal disruption between the integrated circuit and the sensor elements when the conformal sensor device is curved about the surface. In some embodiments, the patterned cutouts include a cutout depth to minimize buckling deformation of the thin-film substrate and to minimize sensor performance degradation of one or more of the plurality of sensor elements when the conformal sensor device is curved about the surface.

While FIG. 5 depicts several segments 304a-304h, a single petal 304e is shown populated with sensor elements 312. However, the sensor array 500 may include sensor elements in any arrangement on any or all of the segments 304a-304h. For example, any one or more of the segments 304a-304h may include sensor elements 312 similar to segment 304e. In some embodiments, at least two segments 304a-304h may include sensor elements 312, etc. to capture a particular hand grip or placement on a ball in which the array 500 is installed.

In some embodiments, the thin-film substrate may include any number of layers, including at least a top foil layer and a bottom foil layer. At least a portion of the layers may include cutouts in the plurality of patterned cutouts that enable adhesion of one layer to another of the plurality of layers. An adhesive material may be exposed through the cutouts in the bottom foil layer, allowing the adhesive to contact and bond to non-sensing portions of the top foil layer that are aligned with the cutouts. This configuration may maintain registration between the layers during deformation of the thin-film substrate when conformed to a surface having non-zero Gaussian curvature, and may prevent separation of the layers at the patterned cutout locations during and after the conforming process.

The working area 508 may include regions designated for active sensing elements within the thin film sensor array. The working area 508 may include sensor elements 312 arranged in non-Cartesian patterns to maintain uniform sensing resolution across curved surfaces. The working area 508 may be defined by petal-shaped segments 304a-304h that may converge at an apex of the surface (e.g., apex of a sphere or other shape). The working area 508 may interact with electrical trace lines 310 to transmit signals to an integrated circuit 416. In some embodiments, the working area 508 may include tessellated sensor arrays with triangular or hexagonal element geometries.

In some embodiments, the PCB 502 may be positioned at a central hub of the sensor array, serving as the primary interface for electrical connections. The connector 504 may extend radially outward from the PCB 502, aligning with the petal-shaped segments 304a-304h to establish electrical pathways for the sensing elements. The overlap 506 may occur at the edges of adjacent petal-shaped segments, where the non-sensing regions are arranged to maintain structural continuity and alignment. The working area 508 may encompass the tessellated sensing elements distributed across the petal-shaped segments, allowing for uniform spatial coverage over the curved surface.

The central PCB 502 may be depicted as a substantially circular element positioned at the geometric center of the assembly 500. The PCB 502 may serve as the primary electrical interface for the sensor array 500, as described elsewhere herein. A vertical bar or traces 504, 505 may extend outward from the PCB region into the petal-shaped segment 304e, aligned with the overlap strip 506, representing the electrical connection path between the centralized routing hub and the individual petal-shaped segments 304a-304h.

The electrical trace lines 310 may be depicted as elongated conductive paths running longitudinally along the petal-shaped segment 304e from the inner region near the ring bus 505 toward the outer portion of the petal. The electrical trace lines 310 may be arranged substantially along the central meridian of the petal-shaped segment 304e and within a predetermined clearance range defined from the perimeter of the petal-shaped segment 304e, consistent with the hierarchical tree-like routing architecture described elsewhere herein. The electrical trace lines 310 may connect the sensor elements 312 within the working area 508 to the ring bus 505 through the connector 504.

FIG. 6 illustrates an example sensor array 600 having an integrated circuit (e.g., PCB 502). The sensor array 600 may include one or more of a thin-film substrate 602 having patterned cutouts 604, petal-shaped segments 606a-606h, electrical trace lines 608, a sensor array 610, a ring bus 612, and an integrated circuit/PCB 614. The thin-film substrate 602 may be formed of flexible material for conforming to curved surfaces. In some embodiments, the thin-film substrate 602 may be the same as or similar to the thin-film substrate 414, as described elsewhere herein.

The patterned cutouts 604 may represent strategically positioned openings to accommodate mechanical strain when the substrate is applied (i.e., contoured) to a surface of an object (e.g., a three-dimensional object). In some embodiments, the patterned cutouts 604 may be the same as or similar to the patterned cutouts 306, patterned cutouts 412, and/or patterned cutouts 604, as described elsewhere herein.

The petal-shaped segments 606a-606h may include discrete regions that may conform to non-developable surfaces. In some embodiments, the petal-shaped segments 606a-606h may be the same as or similar to the petal-shaped segments 304 and/or petal-shaped segments 410, as described elsewhere herein.

The electrical trace lines 608 may represent conductive pathways printed on the thin-film substrate 602 that may couple sensor devices 620 to one or more other sensor devices on substrate 602 and/or to the PCB 614. In some embodiments, the electrical trace lines 608 may be the same as or similar to the electrical trace lines 310 and/or electrical trace lines 406, as described elsewhere herein.

The sensor array 610 may include a collection of sensor elements 620, etc. arranged in a non-Cartesian pattern. In some embodiments, the sensor array 610 may be the same as or similar to the sensor array 112, sensor array 114, and/or sensor array 402, as described elsewhere herein.

The ring bus 612 may represent a centralized routing hub for electrical connections amongst sensor elements 620. In some embodiments, the ring bus 612 may be the same as or similar to the ring bus 408, as described elsewhere herein.

The integrated circuit/PCB 614 may include components to analyze signals from sensors on the sensor array 610. In some embodiments, the integrated circuit/PCB 614 may be the same as or similar to the processor 254, as described elsewhere herein.

In some embodiments, the thin-film substrate 602 may include patterned cutouts 604 positioned between petal-shaped segments 606a-606h to allow for predictable deformation when applied to curved surfaces. The petal-shaped segments 606a-606h may overlap at their edges, creating a tessellated arrangement that accommodates the curvature of the target surface on which the substrate 602 is applied. Electrical trace lines 608 may extend along the surface of the thin-film substrate 602, connecting the sensor array 610 to the integrated circuit 614 through a hierarchical routing architecture.

In some embodiments, the sensor array 610 may be distributed across the petal-shaped segments 606a-606h in a non-Cartesian layout, with sensing elements 620 arranged in tessellated patterns to maintain uniform spatial resolution. The ring bus 612 may serve as a central hub for routing electrical signals from the petal-shaped segments 606a-606h to the integrated circuit 614, which may be positioned at the center of the substrate. The integrated circuit 614 may include conductive connectors that interface with the ring bus 612, allowing for multiplexing and communication between the sensing elements and external systems.

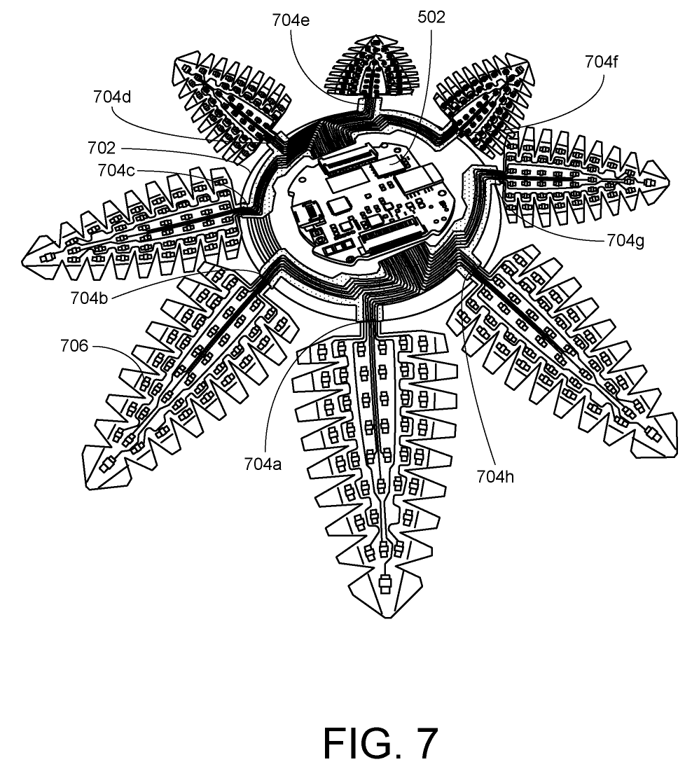

In some embodiments, the grip assessment systems described herein include a first electronic device (e.g., sensor array 112) and a second electronic device (e.g., sensor array 114), each incorporating a thin-film substrate architecture for conformal application to curved surfaces having non-zero Gaussian curvature. Each electronic device 112, 114 may include a thin-film substrate having a centralized hub and a plurality of protrusions (e.g., 606a-606h of FIG. 6) extending radially outward from a hub 308 through a plurality of bridges 704a-704h (FIG. 7). The hub 308 may serve as a routing and connection point for electrical signals, while the protrusions 606a-606h may include discrete petal-shaped segments having sensors that may electrically connect to the ring bus 612 on the hub 308.

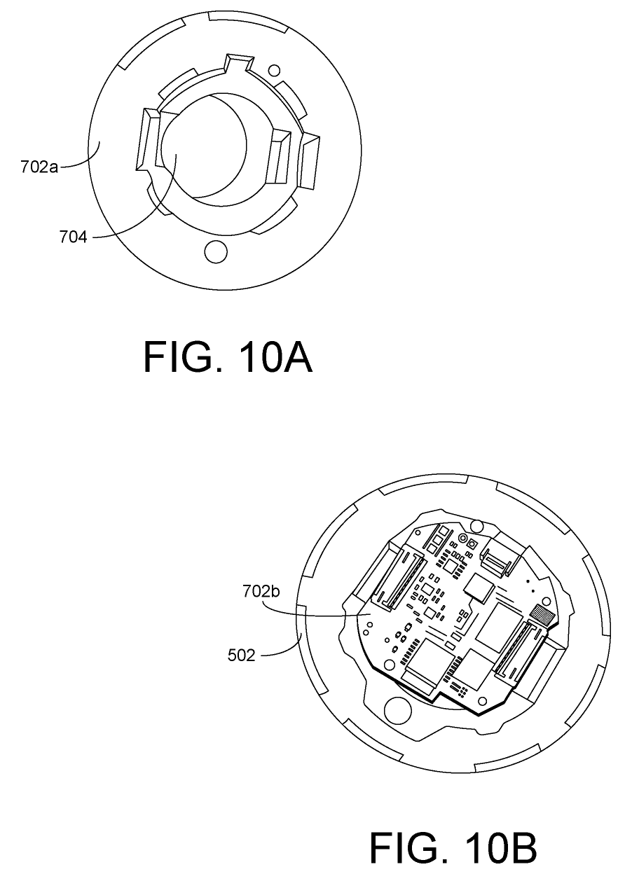

Each protrusion 606a-606h extends from one of the respective adjacent bridges 704a-704h, which are mechanically and electrically coupled to the hub. The bridges 704a-704h function as flexible connection points that allow the protrusions to bend and conform to a target surface while maintaining electrical continuity between the hub 308 and the sensor elements (e.g., sensor element 706 of FIG. 7) disposed on each respective protrusion. The bridges 704a-704h are positioned to minimize mechanical stress concentration during deformation while preserving signal integrity throughout the conformal application process. For example, the bridges 704a-704h lie along the equator and are sandwiched between the hemispheres of the shell (e.g., housings 702a, 702b of FIGS. 10A and 10B), which contributes to preventing deformation during mechanical stress on the object as a whole.

The sensor array 402 disposed on each electronic device 112, 114 may include a plurality of sensor elements 706 arranged in a tessellated pattern across the plurality of protrusions 606a-606h. The sensor elements may be interconnected by a plurality of electrical trace lines that follow hierarchical routing architectures optimized for curved surface applications, as described elsewhere herein. The non-rectangular geometry of the sensor elements, which may include triangular, hexagonal, or other polygonal shapes, enables efficient coverage of curved surfaces compared to conventional Cartesian grid arrangements.

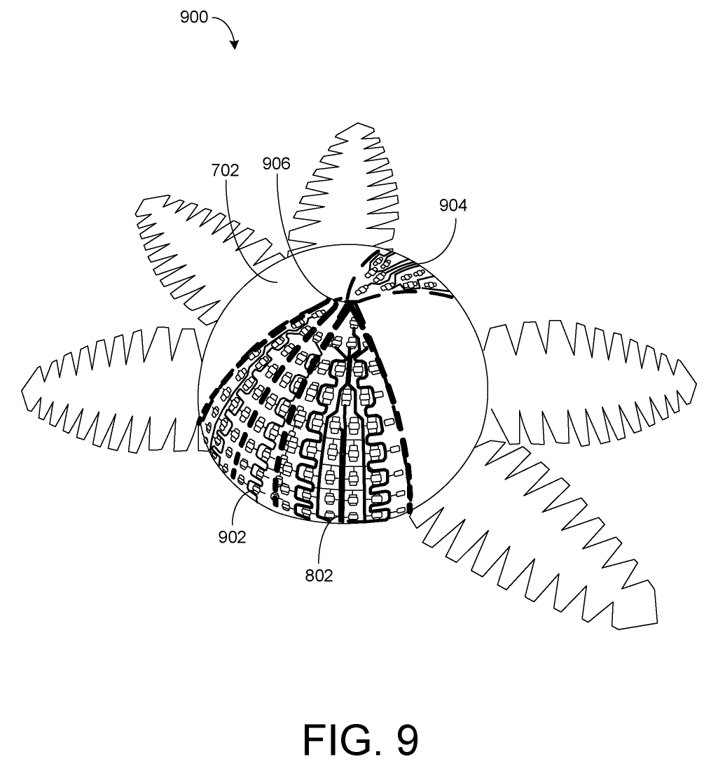

Each protrusion 606a-606h may be arranged to cover a designated portion of a curved surface of a shell (e.g., hemispherical shape 702) when the thin-film substrate is deformed about the respective bridges 704a-704h and coupled to the curved surface. The geometric design ensures that when the protrusions 606a-606h are properly positioned and adhered to the shell, a distal end of each protrusion substantially covers an apex region 906 (FIG. 9) of the shell. This configuration enables comprehensive sensor coverage across the curved surface while maintaining uniform spatial sensing resolution.

The deformation process may include bending each protrusion 606a-606h about its respective bridge connection to the hub 308, allowing the flexible thin-film substrate to conform to the compound curvature of the target shell. The petal-shaped geometry of the protrusions 606a-606h may be mathematically optimized to minimize distortion and buckling when transitioning from a planar configuration to the three-dimensional curved application. This ensures that sensor elements maintain their intended spacing and electrical properties throughout the conforming process.

In some embodiments, the first electronic device 112 is coupled to the second electronic device 114 through a processor (e.g., processor 254 and/or integrated circuit 614 and/or PCB 502) positioned at the respective hubs. The hubs are arranged at the base portions of their respective shells, and when the two electronic devices are assembled together, they may form at least a portion of an inner core of a non-developable three-dimensional shape. This dual-hemisphere configuration enables sensing coverage over a surface of spherical, ellipsoidal, or other complex curved geometries that cannot be developed (flattened) without distortion. The second thin-film substrate may include a second ring bus electrically coupled to the integrated circuit, the second ring bus configured to aggregate signals from the plurality of additional electrical trace lines of the second sensor array and transmit the aggregated signals to the integrated circuit for coordinated processing with signals from the first sensor array.

The processor serves as the central processing unit for both electronic devices (e.g., array 112 and array 114), managing signal acquisition, processing, and communication functions for the grip assessment system. The processor is electrically connected to the sensor arrays of both devices through the hub routing architecture, enabling coordinated data collection and analysis across the complete three-dimensional surface.

The integrated first and second electronic devices 112, 114 may function together through the processor to perform comprehensive analysis of interactions with the outer surface of the non-developable three-dimensional shape. The grip assessment systems described herein may determine multiple types of interactions, including grip characteristics of a user gripping the three-dimensional shape, movement patterns or series of movements of the shape itself, and temporal interaction data collected over specified time periods.

The grip analysis capabilities may include measurement and characterization of grip pressure distribution, finger placement accuracy, grip stability metrics, and individual digit identification. The systems described herein can distinguish between different grip patterns, detect grip anomalies, and calculate performance metrics relevant to sports equipment applications, medical monitoring, or ergonomic assessment. Movement analysis may encompass detection of acceleration, rotation, impact forces, and complex motion patterns that provide insights into equipment usage or user behavior patterns.

The thin-film substrates described herein may incorporate regions patterned with auxetic structures designed to locally deform under mechanical stress while providing global elasticity to the substrate. These auxetic patterns create controlled deformation zones that enhance the overall conformability of the substrate to complex curved surfaces. The auxetic structures function as mechanical metamaterials that exhibit negative Poisson's ratio behavior, expanding laterally when stretched longitudinally, which facilitates improved accommodation of compound curvatures without creating wrinkles or stress concentrations.

The auxetic patterns may be strategically positioned within non-sensing regions of the substrate to avoid interference with sensor element performance while maximizing the mechanical compliance benefits. These structures enable the thin-film substrate of the sensor arrays/devices 112, 114 to achieve greater conformity to highly curved surfaces than would be possible with conventional substrate designs, while maintaining the structural integrity necessary for reliable sensor operation.

The thin-film substrate used for the arrays 112, 114 may further incorporate structural reinforcement layers selectively positioned at areas identified as prone to high mechanical stress during the conforming process. These reinforcement zones may be strategically located at hinge locations where protrusions connect to bridges, at sharp edges where substrate geometry transitions occur, and at small-angle corners where stress concentration is anticipated. The reinforcement layers provide additional material thickness and enhanced tear resistance to prevent mechanical failure during installation or operational use.

The edges of the discrete regions, particularly the protrusion perimeters, may include feathered edge slits (e.g., cutouts 604) that facilitate local strain deformation and reduce buckling when the substrate is adhered to high-curvature regions of the target surface. These feathered edges create graduated flexibility zones that allow smooth transition between the relatively rigid sensor-bearing areas and the highly flexible edge regions that may accommodate the greatest deformation during conformal application.

The electrical routing lines coupling sensor elements may be arranged in a hierarchical tree-like or branched structure optimized for curved surface applications. A central trunk routing line may extend substantially along geodesic paths of minimal curvature, following mathematical paths that minimize mechanical stress during substrate deformation. Branch routing lines extend outward from the central trunk to individually address each sensor element, creating a distributed addressing system that minimizes the total routing length while maintaining signal integrity. This hierarchical architecture reduces the complexity of connections utilized at the central hub while enabling efficient signal multiplexing and communication with external processing systems. The tree-like topology also provides redundancy and fault tolerance, as alternative routing paths can be established if individual traces are damaged during installation or operation.

In some embodiments, the sensor array 402 may incorporate an adhesive layer positioned below a first and second foil layer of the multilayer substrate construction. This adhesive layer may adhere to a non-electrode side of the first foil while simultaneously bonding to non-sensing portions of the second foil that are exposed through strategically positioned patterned cutouts in the first foil. This configuration may maintain alignment between the multiple foil layers and prevents separation during the mechanical deformation occurring when conforming the array 402 to a target surface.

The adhesive layer may extend through pre-cut openings in the substrate to enhance alignment between top and bottom foil layers while providing structural reinforcement that increases tear resistance and mechanical stability. The adhesive configuration ensures that sensor elements in different layers maintain proper registration and electrical contact throughout manufacturing and operational use.

In some embodiments, the sensor elements may be arranged asymmetrically with variable densities to provide increased sensing resolution in targeted sensing areas where interaction frequency is expected to be higher relative to other areas of the surface. This adaptive sensor placement enables the conformal sensor device to allocate sensing resources efficiently across the substantially curved surface, providing fine-grained measurement capability in regions where frequent or high-priority interactions are anticipated while maintaining adequate sensing coverage in regions where interaction frequency is expected to be lower. In some embodiments, the targeted sensing areas having higher relative interaction frequency may include regions of the substantially curved surface that are determined to typically receive digit input, such as fingertip contact zones, palm contact zones, or other anatomically defined grip regions identified through user interaction data or biomechanical analysis. In some embodiments, the variable density arrangement may be determined based on prior grip assessment data, user profile data, or application-specific interaction patterns collected from the sensor array during use. The asymmetric arrangement of sensor elements with variable densities may be implemented across the plurality of petal-shaped segments of the thin-film substrate such that petal-shaped segments positioned in regions of higher expected interaction frequency comprise a greater number of sensor elements per unit area than petal-shaped segments positioned in regions of lower expected interaction frequency, while maintaining substantially uniform sensing coverage across the entire substantially curved surface.

In some embodiments, the variable density arrangement of sensor elements may be implemented by varying the spacing between adjacent sensor elements within individual petal-shaped segments, with sensor elements positioned closer together in petal-shaped segments covering regions of higher expected interaction frequency and farther apart in petal-shaped segments covering regions of lower expected interaction frequency. In some embodiments, the variable density arrangement may be implemented by varying the number of sensor elements disposed on each petal-shaped segment, with petal-shaped segments positioned in regions of higher expected interaction frequency comprising a greater number of sensor elements than petal-shaped segments positioned in regions of lower expected interaction frequency. In some embodiments, the variable density arrangement may be implemented by varying the size of individual sensor elements, with smaller sensor elements providing higher spatial resolution in targeted sensing areas and larger sensor elements providing lower spatial resolution in areas of lower expected interaction frequency.

In general, the sensor arrays may be formed of or on a thin-film, substantially inelastic substrate engineered for application onto curved surfaces having non-zero Gaussian curvature. The substantially inelastic nature of the substrate enables maintenance of dimensional stability of printed sensor elements and electrical routing during and after conforming to complex curved geometries. Unlike elastic substrates that would stretch and potentially cause misalignment of printed components, the substantially inelastic substrate maintains the printed features in precise registration while still providing sufficient flexibility to bend and conform to non-developable surfaces such as spheres, ellipsoids, hyperbolic paraboloids, and other double-curvature geometries.

The substrate materials used herein may include one or more polymers such as polyimide, polyethylene terephthalate (PET), or polyethylene naphthalate (PEN) that exhibit high tensile strength and low elongation characteristics. These materials can withstand the mechanical stresses associated with conforming to surfaces having non-zero Gaussian curvature without experiencing permanent deformation or dimensional changes that would compromise sensor performance. The substantially inelastic properties ensure that conductive trace geometries, sensor element spacing, and electrical connectivity remain stable throughout the conforming process and during operational use.

The sensor arrays described herein may include impedimetric sensor elements that measure changes in electrical impedance in response to physical stimuli such as pressure, temperature, humidity, and/or capacitive coupling. The impedimetric sensors offer advantages in curved surface applications due to their ability to maintain consistent electrical characteristics despite substrate deformation and their compatibility with printed electronics manufacturing processes. The impedimetric sensing mechanism provides stable, repeatable measurements across the curved surface while accommodating the mechanical constraints imposed by non-developable geometries.

In some embodiments, the grip assessment systems described herein may perform comprehensive measurement and analysis of grip characteristics, including detailed grip pressure mapping, precise finger placement detection, and temporal analysis of grip adjustments over time. The systems may utilize advanced signal processing algorithms to distinguish individual digits, analyze grip mechanics, and assess technique quality for applications in sports equipment, medical monitoring, or ergonomic evaluation. The impedimetric sensor data may be processed to extract multiple grip parameters simultaneously, providing a dataset for performance analysis and feedback. The analyzed grip characteristics may be communicated to external devices through integrated wireless communication capabilities of communication engine 240, for example, enabling real-time monitoring, data logging, and performance feedback applications. The communication engine 240 may support multiple protocols and can interface with smartphones, tablets, computers, or specialized monitoring equipment depending on the application requirements. The external communication capability enables the grip assessment systems described herein to function as part of larger monitoring networks or training systems, providing immediate feedback to users or long-term performance tracking for coaches, therapists, or researchers.

The central region of the assembly 600 may include the integrated circuit/PCB 614, which may be visible as a small circular board populated with electronic components including rectangular and cylindrical surface-mount elements. The integrated circuit/PCB 614 may correspond to the PCB 502 described with respect to FIG. 5 and the integrated circuit 416 described with respect to FIG. 4. The integrated circuit/PCB 614 may be positioned at the geometric center of the assembly 600 and may serve as the primary signal processing and routing interface for the sensor array 610.

As shown in FIG. 6, the patterned cutouts 604 may be visible as openings or relief features positioned along the perimeter regions of the petal-shaped segments 606a-606h. The label 604 may point to a region further out along one of the lower-left petal-shaped segments, among the raised sensor elements 620, identifying the patterned cutouts 604 that may enable localized deformation and strain relief when the thin-film substrate 602 is conformed to a curved surface. The patterned cutouts 604 may correspond to the patterned cutouts 306 described with respect to FIG. 3 and the patterned cutouts 412 described with respect to FIG. 4.

FIG. 7 illustrates another view of an assembly 700 of the example sensor array 600 installed on a base portion of a hemispherical shape 702. A second sensor array (not shown) may be a duplicate of sensor array 600 and a second hemispherical shape (not shown) may receive the second sensor array at the base portion (not shown) of the second hemispherical shape. The two arrays may be applied to a surface of the respective hemispheres and then may be coupled together at the base portions to form a spherical sensorized grip assessment system 100, for example. The PCB 502 may be connected to both hemispherical shapes. In some embodiments, each hemispherical shape may use a single PCB 502 coupled to both sensor arrays 600. In some embodiments, each hemispherical shape may use a separate PCB 502 and each PCB 502 may be communicatively and electrically coupled to share data and/or communication protocols.

As shown in FIG. 7, the central hub region of the assembly 700 may include the PCB 502, which may be visible as a component or area near the geometric center of the assembly of sensor array 600. The PCB 502 may correspond to the integrated circuit/PCB 614 described with respect to FIG. 6 and the PCB 502 described with respect to FIG. 5. The PCB 502 may serve as the primary electrical interface and signal processing hub for the sensor array 600 when the assembly 700 is deployed onto a curved surface.

The hemispherical shape 702 may correspond to the spherical shell 302 described with respect to FIG. 3 and may represent the curved surface onto which the assembly 700 may be conformed during deployment. The hemispherical shape 702 may be positioned at the hub of the assembly 700, consistent with the hub-and-petal architecture described elsewhere herein. In this example, eight tapered arms may extend radially outward from the central hub region in an evenly spaced arrangement, forming the overall star-like or petal-like geometry of the assembly 700. The arms 704a through 704h may be positioned around the circumference of assembly 700. Each of the arms 704a through 704h may correspond to one of the bridges described elsewhere herein, about which the petal-shaped protrusions of the thin-film substrate may be deformed when the assembly 700 is applied to the hemispherical shape 702.

Each of the arms 704a through 704h may be densely patterned with repeating small rectangular or square features arranged in rows along the length of each arm. These repeating features may correspond to the sensor elements 706 disposed across the surface of the thin-film substrate 602, consistent with the non-Cartesian tessellated arrangement described elsewhere herein. The sensor elements 706 may be visible near the base of one of the lower-left arms, close to where that arm joins the central hub region, as indicated by the label 706 in FIG. 7.

In some embodiments, each of the arms 704a through 704h may terminate in a pointed tip at its distal end. The pointed tip of each arm may correspond to the distal end of each petal-shaped protrusion that may substantially cover the apex of the hemispherical shape 702 when the assembly 700 is deformed about the respective bridge connections and conformed to the curved surface, as described in greater detail elsewhere herein.

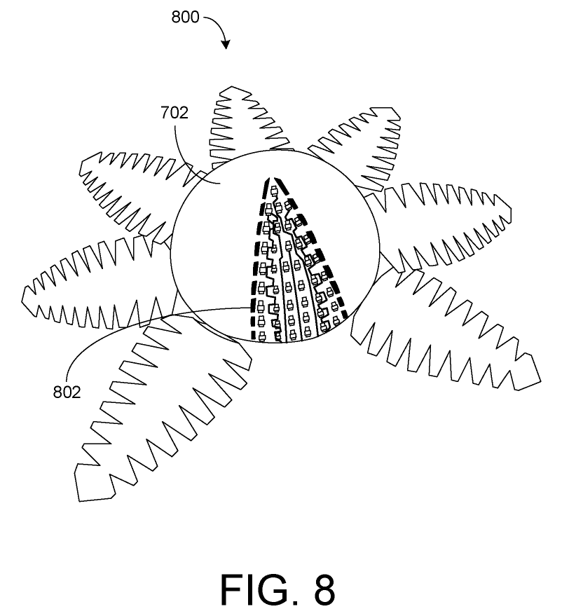

FIG. 8 illustrates a view 800 of the example sensor array 600 installed on base portion 702 and inverted 180 degrees from the view shown in FIG. 7. In this example, the hemispherical shape 702 is shown as portions (e.g., 802) of the array 600 are applied thereto. The portion 802 is a petal-shape with cutouts 604 (FIG. 6), as described elsewhere herein. The patterned cutouts 604 may include strategically positioned openings to allow localized deformation of the thin-film substrate when applied to the hemispherical shape 702 or another shape.

As shown in FIG. 8, the petal-shaped portion 802 may be visible as the patterned central region on a surface of the hemispherical shape 702. The portion 802 may correspond to one of the petal-shaped segments 606a-606h described with respect to FIG. 6 and one of the arms 704a-704h described with respect to FIG. 7, shown in a partially conformed state as it may be applied to the curved surface of the hemispherical shape 702.

FIG. 9 illustrates a view 900 of a sensor array ball 600 of FIG. 8 with additional portions 902 and 904 conformed to the hemispherical shape 702. Each additional portion (e.g., similar to portions 304a-304h and/or 606a-606h) may be conformed or otherwise applied to the hemispherical shape 702 by bending the additional portions of the thin-film substrate to converge a distal end of each petal-shaped segment at an apex 906 of the surface resulting in at least partially overlapping portions of the thin-film substrate to provide substantially uniform sensing on the surface of the hemispherical shape.

FIGS. 10A-10B illustrate example portions of a core for receiving the sensor arrays described herein. FIG. 10A depicts a top hemispherical shape (e.g., a first housing 702a). FIG. 10B depicts a bottom hemispherical shape 702b (e.g., a second housing 702b). The housing 702b may receive the PCB 502, while the housing 702a may include indentations or other structures to enclose and hold a top portion of the PCB 502 when the housing 702a is coupled to (or otherwise assembled with) housing 702b. For example, the housings 702a, 702b may include structural features and/or apertures 704 to accommodate electronic components within the assembly. The housings 702a, 702b may be composed of materials that resist environmental factors such as moisture, temperature changes, or physical impacts. The housings 702a, 702b may include mounting points or slots to secure the PCB 502 and/or other components in place. In some embodiments, the housings 702a, 702b may be cylindrical, rectangular, or spherical, depending on the application of grip assessment.

In some embodiments, the housings 702a, 702b may encase the PCB 502 forming a compact assembly. The housings may include pre-formed cavities or recesses that align with the shape and dimensions of the PCB 502, ensuring proper spatial arrangement of the components. The PCB 502 may be positioned within the housing 702b such that its conductive trace lines extend radially outward, connecting to external sensing elements through apertures or slits in the housing 702b and/or 702a.

As shown in FIG. 10A, the inner cylindrical recess or cavity area of the first housing 702a may include the 704 configured to receive and enclose a top portion of the PCB 502 when the first housing 702a is coupled to the second housing 702b, as described in greater detail with respect to FIG. 10B. The inner circumference of the recess or cavity may include several stepped or contoured surfaces. These stepped surfaces may correspond to the pre-formed cavities or recesses described elsewhere herein that may align with the shape and dimensions of the PCB 502 to ensure a fitted, spatial arrangement of the components when the first housing 702a is assembled with the second housing 702b. A small rounded protrusion may be visible on the front face of the first housing 702a near its lower portion. The rounded protrusion may correspond to a mounting point or alignment feature that may facilitate proper orientation and coupling of the first housing 702a to the second housing 702b during assembly. In some embodiments, more than one small protrusion and/or protrusions of different shapes may be utilized (not shown) to align or facilitate proper orientation and coupling of the first housing 702a to the second housing 702b.

As shown in FIG. 10B, the second housing 702b may represent the outer circular body of the second housing 702b. The second housing 702b may correspond to the second hemispherical shape described elsewhere herein that may receive the PCB 502 within its front-facing surface region. The PCB 502 may be visible as a mounted electronic circuit board positioned on the front face of the second housing 702b. The PCB 502 may correspond to the integrated circuit/PCB 614 described with respect to FIG. 6 and the PCB 502 described with respect to FIG. 5, and may serve as the primary signal processing and routing interface for the sensor arrays 112, 114 when the assembly is deployed onto a curved surface.

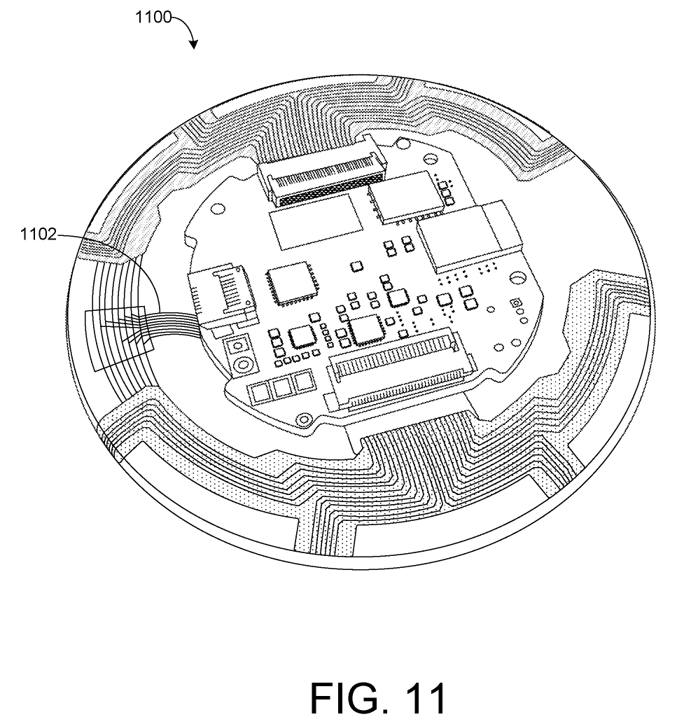

FIG. 11 illustrates a hemispherical shell 702b with a sensor array 114 installed thereon. The PCB 502 is also installed within an aperture of the shell 702b and connected to the sensor array through the trace lines 1102. The hemispherical shell 702b may represent one half of a complete spherical sensor system, such as would be used in a baseball or other sports grip assessment device. The sensor array 114 is shown in its fully conformed state, with the thin-film substrate having successfully deformed from its original planar fabrication geometry to match the compound curvature of the hemispherical shell 702b. The sensor array 114 includes a plurality of petal-shaped segments that extend radially from a central hub region toward the apex of the hemispherical shell 702b.

The PCB 502 is shown installed within an aperture of the hemispherical shell 702b, positioned at the base of the hemisphere to serve as the central processing and routing hub for the sensor array 114. The PCB 502 incorporates a circular or disc-shaped configuration that optimizes the use of available internal space while maintaining the mechanical balance characteristics of spherical applications, such as baseballs. The positioning of the PCB 502 within the shell aperture enables direct electrical connection to the sensor array while providing protection for the electronic components from external environmental factors.

The trace lines 1102 provide electrical interconnection between the PCB 502 and the distributed sensor elements of the sensor array 114. These trace lines 1102 represent the hierarchical tree-like routing architecture described herein, with trunk lines extending along geodesic paths of minimal curvature and branch lines extending to individual sensor elements. The trace lines 1102 may be integrated into the thin-film substrate using multilayer printing techniques, with conductive silver-based inks forming the signal pathways and dielectric insulation layers preventing electrical shorts at crossing points.

The connection interface between the trace lines 1102 and the PCB 502 demonstrates the centralized routing hub concept, where signals from the multiple petal-shaped segments converge at the PCB location for multiplexing, signal conditioning, and digital processing. The trace lines 1102 extend from an origin at individual sensor elements, follow the meridian lines of each petal-shaped segment, and terminate at connection points around the perimeter of the PCB 502. This radial connection arrangement minimizes the routing complexity while ensuring reliable electrical connectivity between the distributed sensor array and the central processing electronics.

The installation configuration shown in FIG. 11 illustrates how the flexible thin-film substrate of sensor array 114 maintains intimate contact with the curved surface of hemispherical shell 702b through the use of adhesive bonding layers and the strategic strain relief provided by the patterned cutouts. The conformal installation eliminates air gaps and ensures consistent sensor-to-surface coupling across the hemispherical surface, which is essential for accurate pressure sensing and grip analysis applications.

The hemispherical shell 702b includes structural features such as an equatorial rim or base portion that provides mechanical coupling points for assembly with a corresponding second hemispherical shell to form a complete spherical sensor system. The shell material and thickness are selected to provide adequate mechanical protection for the sensor array 114 while maintaining the tactile properties, surface characteristics, and weight appropriate for the intended application, whether sports equipment, medical devices, or research instrumentation.

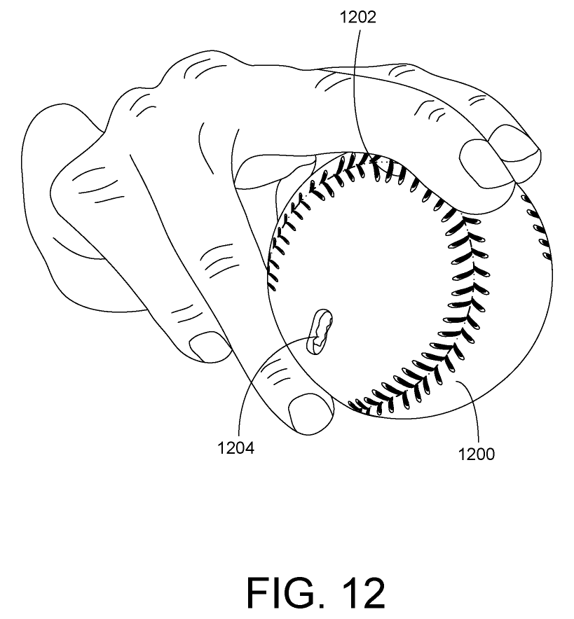

FIG. 12 illustrates a baseball 1200 with one or more embedded sensor arrays (e.g., sensor arrays 112 and/or 114) for assessing a grip of one or more fingers/hand 1202 placed along one or more surfaces of the baseball 1200. The fingers/hand 1202 may represent an interaction with the baseball 1200 during a grip assessment performed by the systems described herein. The baseball 1200 may include a material overlaid onto the sensor arrays. The fingers/hand 1202 may engage one or more of the sensor elements on the sensor arrays to detect pressure, orientation, and finger placement during the interaction. The fingers/hand 1202 may transmit signals to the integrated circuit 416 for analysis of grip characteristics. In some embodiments, the fingers/hand 1202 may be used in conjunction with the grip assessment engine 220 to determine grip strength and finger positioning.

In some embodiments, the fingers/hand 1202 may interact with the baseball 1200 by applying grip pressure to the surface of the baseball 1200. The embedded sensor arrays may measure grip characteristics, such as pressure and finger placement, as the fingers/hand 1202 manipulates the baseball 1200. The baseball 1200 may also include a USB connector 1204 for uploading and/or downloading data.

The USB connector 1204 may provide a wired data interface for uploading and downloading data between the integrated circuit 416 housed within the baseball 1200 and an external computing device. The USB connector 1204 may correspond to the wired communication options described with respect to the communication interface 110, supplementing the wireless communication capabilities of the communication engine 240. In some embodiments, the USB connector 1204 may additionally support charging of a battery housed within the inner core of the baseball 1200, consistent with the power management functions described elsewhere herein. In some embodiments, both the charging and communication capabilities may be wireless, and therefore, such a system will not include a USB connector 1204.

In some embodiments, the surface of the spherical shell 302 or hemispherical shape 702 may serve as a first surface having non-zero Gaussian curvature onto which the thin-film substrate and sensor array may be conformed. An outer layer of material may surround the thin-film substrate conformed to the first surface, forming a second surface of the conformal sensor device.

The second surface may include a cover material such as leather, synthetic rubber, or a composite material applied over the sensor arrays 112, 114 to enclose the thin-film substrate between the first surface and the second surface. In some embodiments, the second surface may correspond to the outer cover of a ball, such as the leather cover of the baseball 1200, which may be applied over the sensor arrays after the sensor arrays are conformed to the hemispherical shells 702a, 702b. The second surface may transmit applied forces, pressures, and interactions from an exterior environment through to the sensor elements of the sensor array, enabling the sensor array to detect grip characteristics, finger placement, and other interactions occurring at the outer surface of the assembled device. In some embodiments, the second surface may surround the thin-film substrate conformed to the first surface, enclosing the sensor array between the first surface and the second surface such that forces applied to the second surface are transmitted through to the sensor elements of the sensor array.

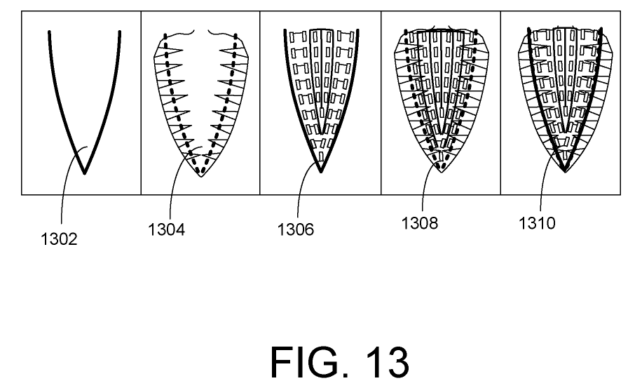

FIG. 13 illustrates example layers of a petal of the sensor arrays described herein. A first layer 1302 may represent a base without wings. A second layer 1304 may represent a base with wings. A third layer 1306 may represent a petal bottom layer. A fourth layer 1308 may represent a petal top layer. A fifth layer 1310 may represent a combined view of the top and bottom of a petal.

The first layer 1302 may include a foundational structure designed to support the arrangement of petal block layers. The first layer 1302 may include a flat or contoured surface that may serve as a stable platform for the attachment of other components. The first layer 1302 may include features that may assist in maintaining the alignment of petal block layers during assembly. The first layer 1302 may interact with the third layer 1306 to ensure proper positioning of sensor elements. In some embodiments, the first layer 1302 may be replaced with a modular base structure that may accommodate varying configurations of petal block layers.

The layer 1304 may include extensions that may assist in the alignment of petal block layers. The layer 1304 may include wing-like protrusions that may extend outward to provide additional surface area for alignment purposes. The layer 1304 may include features that may stabilize the petal block layers during assembly or operation. The layer 1304 may interact with the layer 1308 to ensure proper overlay of sensor components. In some embodiments, the layer 1304 may be substituted with a base structure that may include adjustable extensions to accommodate different sensor configurations.

The third layer 1306 may include a substrate layer that may serve as the base for sensor elements. The third layer 1306 may include a thin-film substrate that may include patterned cutouts to allow for localized deformation. The third layer 1306 may include features that may enable adhesion to the first layer 1302 or the base with wings. The third layer 1306 may interact with electrical trace lines to establish connections between sensor elements and the integrated circuit/PCB 502. In some embodiments, the third layer 1306 may include additional reinforcement layers to reduce mechanical stress during operation.

The layer 1308 may include an overlay layer that may house additional sensor components or protective features. The layer 1308 may include a thin-film substrate that may include patterned cutouts to align with the petal bottom layer 1306. The layer 1308 may include features that may shield sensor elements from environmental factors. The layer 1308 may interact with an adhesive backing layer to maintain alignment with the petal bottom layer 1306. In some embodiments, the layer 1308 may include thermo-shrink material to improve conformity to curved surfaces.

The layer 1310 may represent a combined view of the third layer 1306 and the petal top layer 1308. The layer 1310 may include a representation that may illustrate the alignment and interaction of sensor elements across both layers. The layer 1310 may include features that may assist in determining the spatial arrangement of sensor components. The layer 1310 may interact with the central hub 308 to facilitate signal routing from sensor elements to the integrated circuit 1006. In some embodiments, the layer 1310 may include additional visual markers to highlight areas of active and inactive sensor regions.

In some embodiments, the first layer 1302 may serve as the foundational layer of the sensor array, providing structural support for subsequent layers. The layer 1304 may overlay the first layer 1302, introducing extended regions that may accommodate mechanical strain relief or adhesion points. The third layer 1306 may be positioned above the layer 1304 and may include patterned cutouts designed to deform predictably under stress.

In some embodiments, the layer 1308 may be arranged above the third layer 1306 and may feature fewer structural lines to allow for localized flexibility. The layer 1310 may illustrate the alignment and interaction between the third layer 1306 and the layer 1308, showing how the overlapping regions may contribute to the overall mechanical stability of the sensor array.

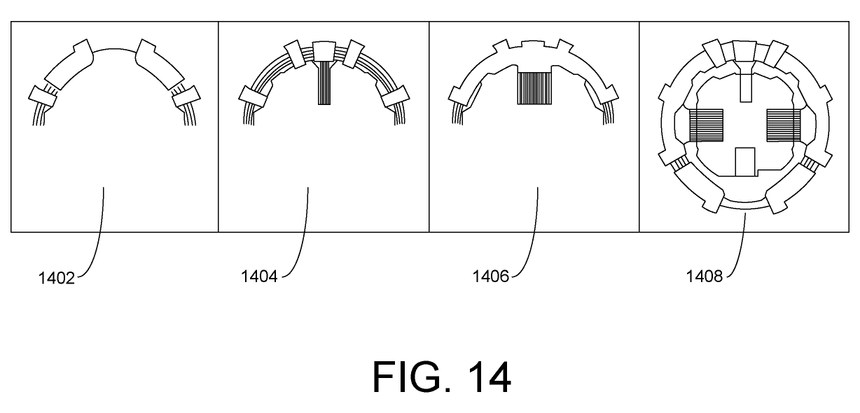

FIG. 14 illustrates connector block layers of the hub described herein. The connector block layers may include a bottom layer without a PCB connector 1402, a bottom layer with the PCB connector 1404, a top layer 1406, and a combined view with the PCB outline. The PCB connector may represent an electrical interface for connecting sensor arrays to processing units and/or sensor elements. In some embodiments, the PCB connector may be the same as or similar to the electrical connectors 1108, sensor interface 108, or sensor interface 208, as described elsewhere herein. The PCB outline may define the physical boundaries of the PCB 502 for integration with the connector block layers.

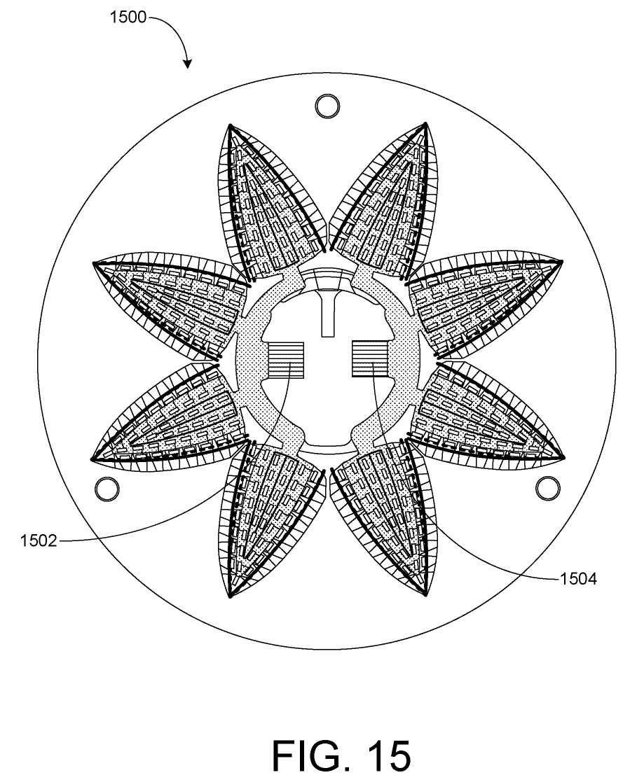

FIG. 15 illustrates an example system 1500 for a thin film sensor array for grip assessment on curved surfaces. The system 1500 depicts a combination of layers shown in FIGS. 13 and 14. Connector 1502 may connect a first array 112 to the PCB 502 while connector 1504 may connect the second array 114 to the PCB 502. As shown in FIG. 15, the connector 1502 may correspond to the electrical interface through which the first sensor array 112 may be coupled to the PCB 502. The connector 1502 may represent one of the connection pathways described with respect to the connector 504 of FIG. 5, through which row-specific electrical traces from the petal-shaped segments of the first sensor array 112 may be routed to the ring bus for multiplexing and signal transmission to the integrated circuit.

The connector 1504 may correspond to the electrical interface through which the second sensor array 114 may be coupled to the PCB 502. The connector 1504 may represent a second connection pathway through which row-specific electrical traces from the petal-shaped segments of the second sensor array 114 may be routed to the ring bus for multiplexing and signal transmission to the integrated circuit. The opposing orientation of connectors 1502 and 1504 relative to the central opening may correspond to the dual-hemisphere configuration described with respect to FIGS. 10A, 10B, and 11, in which the first sensor array 112 and the second sensor array 114 may each connect to the PCB 502 from opposite sides of the hub to form the complete spherical sensor device 400.

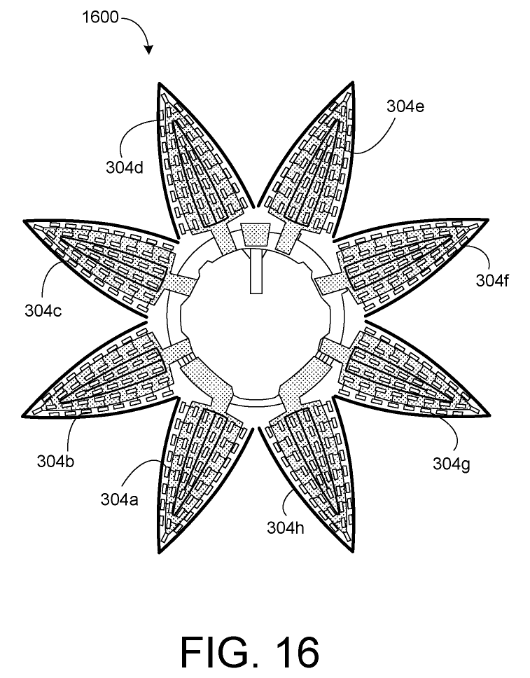

FIG. 16 illustrates an opposite side 1600 of the example system 1500. The side 1600 may correspond to a reverse or back-facing view of the assembled multilayer thin-film substrate structure 1500 described with respect to FIG. 15, showing the underside of the hub-and-petal assembly prior to conforming to a curved surface. A number of trace lines to the petals 304a-304h are coupled to connector 504 around the hub 308.

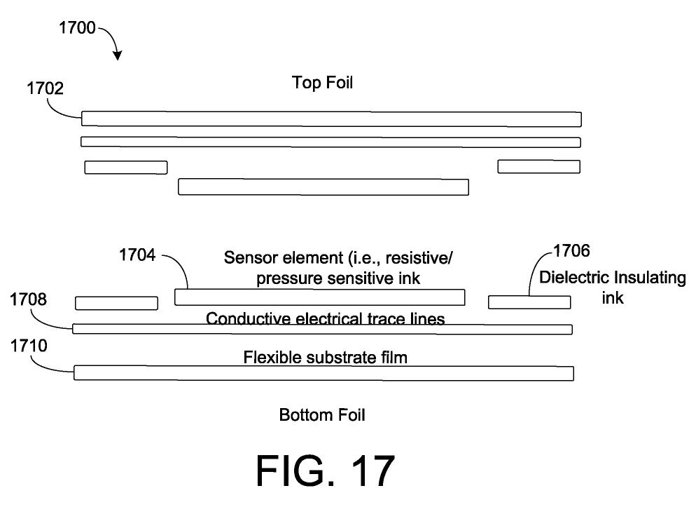

FIG. 17 illustrates a sensor cross-section 1700 of an example sensor array. The sensor cross-section 1700 may include one or more of a thin film sensor foil 1702, a sensor element 1704, a dielectric insulating ink 1706, conductive electrical trace lines 1708, a flexible substrate film 1710, and/or other components. The cross-sectional layer structure depicted in FIG. 17 may correspond to the multilayer petal construction illustrated in FIG. 13, wherein the petal bottom layer 1306 and petal top layer 1308 of FIG. 13 collectively represent the layered assembly of the thin film sensor foil 1702, sensor element 1704, dielectric insulating ink 1706, conductive electrical trace lines 1708, and flexible substrate film 1710 shown in cross-section in FIG. 17.

The thin film sensor foil 1702 may include a flexible layer for conforming to curved surfaces. The thin film sensor foil 1702 may include a polymeric substrate such as polyethylene terephthalate (PET), polyethylene naphthalate (PEN), or polyimide (PI) with a thickness ranging from about 25 microns to about 125 microns. The thin film sensor foil 1702 may serve as a base for printed conductive and dielectric layers, which may be arranged in a multilayer architecture. The thin film sensor foil 1702 may interact with the adhesive backing layer 908 to securely bond the sensor array to a curved surface. In some embodiments, the thin film sensor foil 1702 may include thermo-shrink material selectively activated through localized heat application to conform to highly curved surfaces when a portion of the substrate or foil 1702 is composed of heat shrink material. The thin film sensor foil 1702 may correspond to the foundational substrate material of the petal bottom layer 1306 depicted in FIG. 13, representing the flexible polymeric base upon which the conductive electrical trace lines 1708, dielectric insulating ink 1706, and sensor elements 1704 are sequentially deposited during the manufacturing process described herein. The sensor element 1704 may represent a printed component capable of detecting physical interactions.

The dielectric insulating ink 1706 may provide electrical isolation between overlapping conductive layers. The dielectric insulating ink 1706 may include UV-curable polyurethane-acrylate ink applied at junctions where conductive electrical trace lines 1708 intersect. The dielectric insulating ink 1706 may be printed in bridge-like structures to prevent electrical shorts between layers. The dielectric insulating ink 1706 may interact with conductive electrical trace lines 1708 to maintain signal integrity across the sensor array. In some embodiments, the dielectric insulating ink 1706 may be applied in dimensions of about 1.5 millimeters by about 1.0 millimeters with a thickness of about 20 microns to about 30 microns. The dielectric insulating ink 1706 may correspond to the insulating bridge structures represented within the petal bottom layer 1306 and petal top layer 1308 of FIG. 13, providing the electrical isolation between conductive layers that enables the multilayer routing architecture of the assembled petal-shaped segment depicted in the combined view of the fifth layer 1310 of FIG. 13.

The conductive electrical trace lines 1708 may include pathways for transmitting signals across the sensor array. In some embodiments, the conductive electrical trace lines 1708 may be the same as or similar to the electrical trace lines 406, 608, 710, 806, 904, 1104, 1512, and/or 1612, as described herein. The conductive electrical trace lines 1708 may correspond to the conductive routing paths represented within the dense lattice pattern of the petal bottom layer 1306 of FIG. 13, extending substantially along the central meridian of each petal-shaped segment toward the central hub 308 within a predetermined clearance range defined from the perimeter of each respective petal-shaped segment. The flexible substrate film 1710 may serve as a base layer supporting the overall structure of the sensor array. The flexible substrate film 1710 may correspond to the foundational substrate layer represented by the tapered outer boundary of the first layer 1302 of FIG. 13, providing the primary mechanical support and flexibility that enables the assembled petal-shaped segment to conform to surfaces having non-zero Gaussian curvature as depicted in the sequential layer progression of FIG. 13 from the first layer 1302 through the combined fifth layer 1310.

In some embodiments, the thin film sensor foil 1702 may be positioned as the topmost layer in the assembly, with the sensor element 1704 embedded within or directly beneath it, corresponding to the petal top layer 1308 of FIG. 13 overlying the petal bottom layer 1306 in the assembled multilayer construction. The dielectric insulating ink 1706 may be applied in discrete regions to act as a spacer between the thin film sensor foil 1702 and the conductive electrical trace lines 1708, preventing electrical shorts while maintaining alignment, consistent with the bridge structures represented within both the petal bottom layer 1306 and petal top layer 1308 of FIG. 13. The conductive electrical trace lines 1708 may be printed in a patterned layout on the flexible substrate film 1710, which may serve as the foundational layer providing mechanical support and flexibility, corresponding to the base substrate layer 1302 of FIG. 13 upon which the subsequent layers of the petal construction are sequentially deposited.

In some embodiments, the sensor element 1704 may be electrically connected to the conductive electrical trace lines 1708, allowing signals to propagate through the layered structure, consistent with the active sensing regions represented within the dense lattice pattern of the petal bottom layer 1306 and petal top layer 1308 of FIG. 13. The dielectric insulating ink 1706 may create isolated pathways for the conductive electrical trace lines 1708, enabling the routing of electrical signals without interference, corresponding to the insulating bridge structures that enable the multilayer routing architecture visible in the combined fifth layer 1310 of FIG. 13. The flexible substrate film 1710 may accommodate the mechanical deformation of the assembly, allowing the thin film sensor foil 1702 and the sensor element 1704 to maintain functional alignment during application to curved surfaces, consistent with the conformable substrate behavior depicted in the progression from the planar petal construction of FIG. 13 to the fully conformed configuration illustrated in FIG. 9.

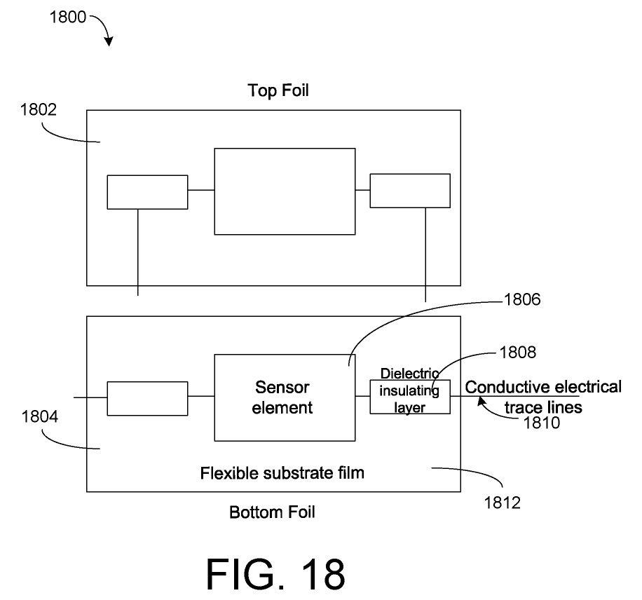

FIG. 18 illustrates an example of sensor foil layers 1800 for the sensor elements described herein. The sensor foil layers 1800 may include a top foil 1802 and a bottom foil 1804. The top foil may overlay the bottom layer 1804. The bottom layer 1804 may include a sensor element 1806, conductive electrical trace lines 1808, dielectric insulating ink 1810, and a flexible substrate 1812.

The top foil 1802 may include a flexible layer designed to conform to curved surfaces. In some embodiments, the top foil 1802 may be the same as or similar to the thin film sensor foil 1702, as described herein.

The sensor element 1806 may represent a printed component capable of detecting physical interactions. In some embodiments, the sensor element 1806 may be the same as or similar to the sensor element 1704 and/or sensor elements 312, 404, 804, and/or 912 as described herein.

The dielectric insulating ink 1808 may provide electrical isolation between overlapping conductive layers. In some embodiments, the dielectric insulating ink 1808 may be the same as or similar to the dielectric insulating ink 1706, as described herein.

The conductive electrical trace lines 1810 may include pathways for transmitting electrical signals across the sensor foil. In some embodiments, the conductive electrical trace lines 1810 may be the same as or similar to the conductive electrical trace lines 1708, as described herein.

The flexible substrate 1812 may include a base material that supports the integration of sensor components. In some embodiments, the flexible substrate 1812 may be the same as or similar to the thin-film substrate 414, 602, 702, 810, 902, and/or 1102, as described elsewhere herein.

In some embodiments, the top foil 1802 may include a layered configuration where the sensor element 1806 is positioned centrally within the foil to interact with conductive electrical trace lines 1810. The conductive electrical trace lines 1810 may extend outward from the sensor element 1806 and traverse the flexible substrate 1812, forming a network of connections that may facilitate signal transmission. Dielectric insulating ink 1808 may be applied at crossing points of the conductive electrical trace lines 1810 to prevent electrical shorts and maintain isolation between intersecting layers.

In some embodiments, the flexible substrate 1812 may serve as a base layer that supports the arrangement of the sensor element 1806, conductive electrical trace lines 1810, and dielectric insulating ink 1808. The flexible substrate 1812 may include predefined regions that allow the top foil 1802 to conform to curved surfaces without excessive strain. The dielectric insulating ink 1808 may form insulating bridges that permit the conductive electrical trace lines 1810 to overlap across different layers of the top foil 1802 without causing electrical interference.

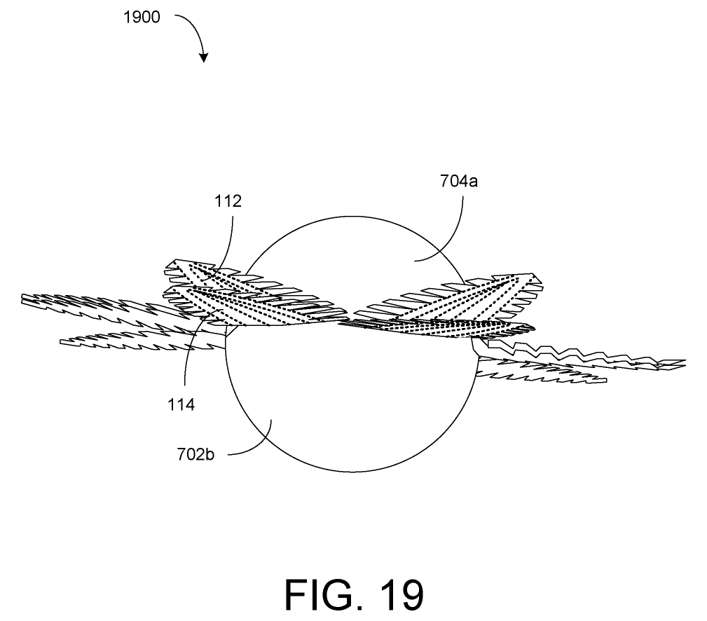

FIG. 19 illustrates an example spherical object with two sensor arrays. The spherical object may include the first spherical portion 702a (FIG. 7) and the second spherical portion 702b (FIG. 7). Joining the portion 702a to the portion 702b may result in one or more slits, orifices, or gaps at the equator of the sphere which may be used to receive routing lines and connectors on array 112 and array 114 to connect to a single PCB 502 (not shown) inside the core of the spherical object.

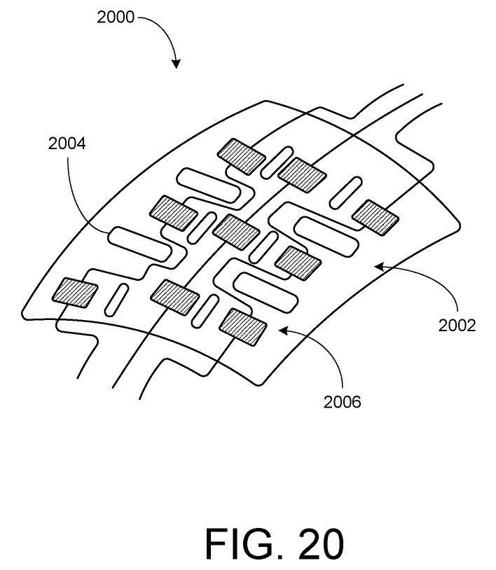

The assembly 1900 may represent the spherical sensor device 400 in a partially assembled state, with the thin-film substrate of each of the two sensor arrays in the process of being conformed to their respective hemispherical shells. The spherical body may be positioned approximately at the center of the assembly 1900, with the equatorial region of the sphere visible between the two hemispherical portions being assembled. The first hemispherical shell may correspond to the first housing 702a described with respect to FIG. 10A, and the second hemispherical shell may correspond to the second housing 702b described with respect to FIG. 10B. FIG. 20 illustrates an example sensor foil design 2000 for the sensor arrays described herein. The sensor foil design 2000 may include one or more of a substrate 2002, holes 2004, and sensor elements 2006. In some embodiments, each of the sensor elements may be printed on the thin-film substrate 2002 with a first discrete sensor element having a rectangular shape overlaid with a second discrete sensor element having a rectangular shape and oriented 90 degrees offset from a centerline of the first discrete sensor element, as shown by sensors 404 (FIG. 4).

The substrate 2002 may include a flexible thin-film material designed to conform to curved surfaces. The substrate 2002 may include materials such as polyimide, polyethylene terephthalate, or polyethylene naphthalate, which may exhibit flexibility while resisting stretching. The substrate 2002 may maintain dimensional stability under thermal and mechanical stress. In some embodiments, the substrate 2002 may include patterned cutouts or edge slits to locally relieve mechanical strain and reduce buckling deformation. The substrate 2002 may be used in conjunction with an adhesive backing layer to securely bond the sensor arrays described herein to a curved surface.

The holes 2004 may represent strategically positioned cutouts within the substrate 2002 to locally relieve mechanical strain. The holes 2004 may include geometric patterns such as auxetic slits or edge feathering, which may allow controlled deformation of the substrate 2002. The holes 2004 may be positioned in non-active areas of the substrate 2002 to avoid interference with sensor elements on the sensor arrays described herein. In some embodiments, the holes 2004 may be created using laser cutting or die cutting techniques to achieve precise dimensions. The holes 2004 may be used in combination with patterned cutouts to enhance the conformability of the thin-film substrate 2002 to surfaces with non-zero Gaussian curvature. For example, in FIG. 20, the holes 2004 are ellipse shaped (e.g., elongated oval or lengthened ovular shaped) cutouts that may allow for further conformation without deformation of the substrate 2002. However, in some embodiments, the holes 2004 may include any shaped cutouts that do not affect sensor data, but allow for an improved conforming to the shape of the surface.

The sensor elements 2006 may include non-Cartesian geometries arranged to maintain uniform spatial sensing resolution across curved surfaces. In some embodiments, the sensor elements 2006 may be the same as or similar to the sensor elements 404 and/or sensor elements 312, as described elsewhere herein.

In some embodiments, the substrate 2002 may include a network of holes 2004 that are distributed across areas not occupied by sensor elements 2006. The holes 2004 may be arranged in a pattern that corresponds to regions of the substrate 2002 experiencing strain relief during deformation. The sensor elements 2006 may be positioned on discrete islands within the substrate 2002, with the surrounding areas of the substrate 2002 forming bridges that connect these islands.

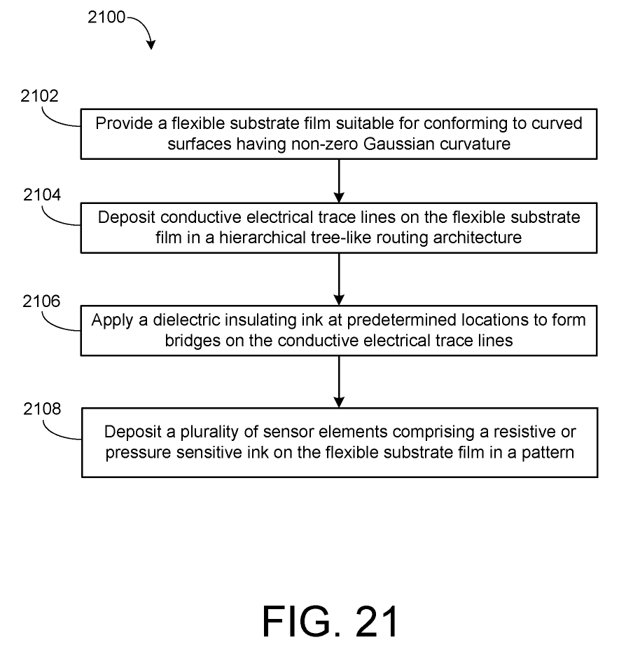

In some embodiments, the holes 2004 may extend through the thickness of the substrate 2002, creating negative spaces that allow the substrate 2002 to flex and stretch in response to mechanical stress. The sensor elements 2006 may be electrically connected through conductive traces that span the bridges of the substrate 2002, maintaining signal transmission while accommodating the mechanical deformation of the substrate 2002. The arrangement of the holes 2004 relative to the sensor elements 2006 may allow the substrate 2002 to conform to complex three-dimensional geometries without disrupting the alignment of the sensor elements 2006.FIG. 21 illustrates a flowchart illustrating a process 2100 for manufacturing a thin film sensor array for curved surface applications. The sensor array (e.g., sensor array 112 or sensor array 114) may be formed for application on non-developable, curved surfaces such as spheres, ellipsoids, or complex 3D geometries. The process 2100 addresses the conventional challenges of applying inelastic thin-film electronics to surfaces with double curvature by introducing petal-based geometries, strain-relief patterns, and electrical routing. The sensor array may include non-rectangular, often tessellated impedimetric sensor elements (e.g., pressure, temperature, and/or capacitive sensors) distributed across petal-shaped regions of an inelastic substrate. These regions are engineered to deform predictably when applied to a curved form, enabling uniform spatial coverage with minimal wrinkling or distortion. The process 2100 may be implemented to produce the multi-foil architecture described herein for conformal sensor devices having petal-shaped segments and patterned cutouts.

At block 2102, the process 2100 may include providing a flexible substrate film suitable for conforming to curved surfaces having non-zero Gaussian curvature. The flexible substrate film may include a polymeric material such as polyethylene terephthalate (PET), polyethylene naphthalate (PEN), or polyimide (PI) with thickness ranging from about 25 microns to about 125 microns. In some embodiments, the substrate film may be prepared by cleaning with isopropyl alcohol to remove contaminants that could interfere with subsequent deposition steps.

At block 2104, the process 2100 may include depositing conductive electrical trace lines including silver ink on the flexible substrate film. The conductive electrical trace lines may be screen-printed using silver-based conductive ink with line widths of about 100 microns to about 250 microns and a thickness of about 8 microns to about 15 microns. The trace lines may be arranged in a tree-like routing architecture with a central trunk extending along geodesic paths and branch lines extending to connect individual sensor locations. The deposited trace lines may be thermally cured at about 120° C for about 30 minutes to ensure proper adhesion and conductivity.

At block 2106, the process 2100 may include applying a dielectric insulating ink at predetermined locations to form bridges on the conductive electrical trace lines. The dielectric insulating ink may include a UV-curable polyurethane-acrylate material applied through micro-dispensing techniques at junctions where trace lines intersect or cross. The insulating bridges may have dimensions of about 1.5 millimeters by 1.0 millimeters with thickness of about 20 microns to about 30 microns. The dielectric ink may be cured using UV radiation at 385 nm for about 60 seconds to achieve proper electrical isolation.

At block 2108, the process 2100 may include depositing a plurality of sensor elements including a resistive or pressure sensitive ink on the flexible substrate film. The plurality of sensor elements may be screen-printed using carbon-loaded polymer ink with a resistance range of 500 ohms to 10 kiloohms in a substantially rectangular pattern of about 8 millimeters by about 5 millimeters with a thickness of about 12 microns to about 20 microns. The plurality of sensor elements may be positioned to overlap with and electrically connect to portions of the conductive electrical trace lines while maintaining electrical isolation through the dielectric insulating ink bridges. In some embodiments, the dielectric insulating ink bridges allow the conductive electrical trace lines to overlap on top and bottom portions of the plurality of sensor elements without causing an electrical short. In some embodiments, the plurality of sensor elements are arranged to maintain substantially uniform spatial sensing resolution when the flexible substrate film is conformed to a curved surface.

In some embodiments, the process 2100 may optionally include curing the deposited materials. This curing process may be over any number of temperatures and times to achieve proper bonding and pressure sensitivity performance. However, in some embodiments, the curing process occurs at about 130° C for about 45 minutes. The curing process may result in pressure sensitivity performance of about 0.5 to about 5% resistance change per 100 grams of applied force, making the sensor suitable for grip assessment applications.

In some embodiments, the process 2100 may optionally include forming patterned cutouts in the flexible substrate film using laser cutting or die cutting techniques to create the strain relief features necessary for conforming to curved surfaces. The cutouts may be positioned in non-active areas to avoid interference with the sensor elements and conductive trace lines. The dielectric insulating ink bridges formed in block 2106 allow the conductive electrical trace lines to overlap on top and bottom portions of the sensor elements without causing an electrical short, enabling the multilayer routing architecture used for complex curved surface applications. This manufacturing method produces individual foil layers that may subsequently be assembled into the multi-foil architecture with adhesive layers as described below.

The process 2100 uses precise alignment and robust bonding of multilayer sensor foils to ensure consistent sensor performance on a curved surface. Surfaces with significant radius of curvature introduce differential deformation that may lead to warping, misalignment, and buckling between the top and bottom foil layers of the thin film sensor array. These deformation effects can compromise the electrical connectivity between conductive electrical trace lines and sensor elements, particularly in pressure-sensitive applications where consistent contact area results in accurate measurements. To mitigate these issues, the process 2100 incorporates a dedicated adhesive layer into the multilayer architecture of the conformal sensor devices. The adhesive layer improves physical adhesion to the curved surface and enhances the structural coherence of the sensor array disposed on the thin-film substrate. The adhesive layer may be strategically positioned to maintain alignment between multiple foil layers while accommodating the mechanical strain relief provided by patterned cutouts and edge slits in the petal-shaped segments.

An example construction for the multi-foil architecture generated by process 2100 may include a bottom foil layer having pre-cut openings or slits positioned at predetermined locations. The bottom foil layer may include the flexible substrate film described in the manufacturing process 2100, with conductive electrical trace lines comprising silver ink deposited thereon and sensor elements comprising resistive or pressure sensitive ink positioned to overlap with and electrically connect to portions of the conductive electrical trace lines. A double-sided adhesive film or transfer tape may be placed beneath the bottom foil layer to provide secure bonding to the curved surface.

A top foil layer may be aligned with the bottom foil layer such that its non-sensing areas contact the adhesive exposed through the cutouts of the bottom layer. The top foil layer may similarly include conductive electrical trace lines and sensor elements manufactured according to the deposition method, with dielectric insulating ink applied at predetermined locations to form bridges on the conductive electrical trace lines. The dielectric insulating ink bridges allow the conductive electrical trace lines to overlap on top and bottom portions of the sensor elements without causing an electrical short, ensuring proper electrical isolation between the multilayer routing architecture. This multi-foil construction provides several advantages for conformal sensor devices applied to curved surfaces. First, the adhesive layer provides anchoring to the substrate by firmly securing the sensor foil assembly to the underlying 3D surface, accommodating complex shapes without peeling or lifting during deformation of the petal-shaped segments. Second, the exposed adhesive through bottom-layer cutouts ensures layer alignment and registration between sensor elements in the top and bottom foils, which is particularly important for pressure-sensitive sensors like force-sensitive resistors that utilize consistent overlap areas. Third, the adhesive layer provides mechanical reinforcement by acting as a structural layer that increases tear resistance and reduces local deformation during handling, installation, or thermal cycling of the conformal sensor device.

The adhesive layer is strategically positioned beneath the bottom foil layer rather than between the top and bottom sensor foils, where it might interfere with electrical contact or sensor response. This configuration preserves precise contact between electrodes in the multilayer sensor array and maintains sensitivity across the tessellated sensor elements. The adhesive layer may extend through pre-cut openings to enhance alignment between the top and bottom foil layers while providing structural support for the tree-like electrical routing architecture that connects the sensor elements to the centralized routing hub or ring bus.

The manufacturing process 2100 for the multi-foil architecture may include forming the cutouts in the bottom foil layer using laser cutting or die cutting techniques after depositing the conductive electrical trace lines and sensor elements. The adhesive film may then be applied beneath the bottom foil layer, with portions of the adhesive exposed through the pre-cut openings. The top foil layer may be aligned and laminated to the bottom foil layer, ensuring proper registration of the sensor elements and electrical connections while maintaining the structural integrity when conforming to surfaces having non-zero Gaussian curvature.

It should be noted that the processes described herein describe possible embodiments, and that the operations and the steps may be rearranged or otherwise modified and that other embodiments are possible. Furthermore, aspects from two or more of the described processes may be combined.

In some embodiments, the conformal sensor technology described herein may utilize a continuous helical-shaped film for application to cylindrical core structures such as tennis racket handles, racquetball handles, golf club grips, baseball bats, or other elongated sporting equipment, tool, or device. The helical film may employ a single continuous thin-film substrate that follows a predetermined helical path around the cylindrical core. This helical geometry provides seamless sensor coverage across all or a portion of a grip region while eliminating the interconnection complexity associated with multiple discrete segments.

The helical film may be manufactured with a predetermined pitch angle and width calculated to provide circumferential coverage of the cylindrical core with controlled overlap zones that ensure continuous surface sensing without sensing gaps. The helical path length and spiral geometry may be modified for a specific core diameter and length of different equipment types, enabling customization for tennis rackets, golf clubs, or other handle-based applications while maintaining manufacturing efficiency through parametric design variations.

In some embodiments, the helical-shaped film incorporates the same thin-film substrate technology described for spherical applications herein, but modified for helical deformation characteristics. The cutout patterns may be oriented to accommodate the stress distribution that occurs when the helical film transitions from a planar manufacturing state to a wrapped configuration around the cylindrical core. Unlike the radial stress patterns encountered in spherical applications, the helical film experiences primarily circumferential and longitudinal strain components that are alleviated by the strain relief geometries in the helical-shaped film described herein.

In some embodiments, sensor elements may be distributed throughout the helical film in tessellated patterns that maintain uniform spatial density when the film is applied to the cylindrical surface. The sensor element orientation may be aligned with the helical path direction, oriented perpendicular to create circumferential sensing bands, or arranged in complex angular patterns that provide multi-directional sensitivity for comprehensive grip analysis.

An example manufacturing process for helical films may utilize specialized substrate cutting and forming techniques to create the continuous helical geometry from planar thin-film materials. The helical film may be produced through precision cutting of wide substrate sheets using computer-controlled cutting systems that follow mathematically defined helical patterns, or through direct printing onto pre-formed helical substrates. The deposition of conductive electrical trace lines, dielectric insulating ink, and sensor elements follows the same screen-printing and curing processes described for spherical applications, with modifications to accommodate the helical substrate geometry and the continuous nature of the electrical routing paths. The helical film enables simplified electrical routing architectures compared to segmented approaches, as the continuous substrate provides uninterrupted pathways for trace lines that follow the helical path without requiring inter-segment connections.

For tennis racket handle applications, the helical film may be applied beginning at a handle base and spiraling upward toward the racket head connection, providing continuous sensor coverage across all or a portion of the grip region. The helical pitch may be optimized to ensure that grip interactions at any rotational position around the handle circumference are detected by sensor elements within the helical film. The continuous coverage enables detection of grip rotation during stroke preparation, grip pressure variations during different phases of stroke execution, and grip stability during ball impact events.

The helical film configuration may provide for detecting grip rotation because the continuous sensor array can detect minor or major rotational movements and grip adjustments that occur during complex stroke sequences. The helical pattern may naturally accommodate the biomechanical aspects of tennis grip techniques, providing sensing coverage that aligns with the natural hand positions and movement patterns used in forehand, backhand, and serve motions, for example.

Golf club grip applications may utilize helical films with pitch angles and coverage patterns to analyze golf grip biomechanics and golf swing mechanics. The helical film may spiral around the grip core with a pitch that ensures comprehensive coverage of finger and palm contact areas during different phases of the golf swing. The continuous sensor coverage enables detection of grip pressure redistribution during swing transitions, identification of grip consistency patterns across multiple swings, and analysis of grip technique variations for different shot types. The helical film design may accommodate the varying grip diameters commonly used in golf club design, from standard grips to oversized or undersized variations, through parametric adjustment of the helical pitch and film width. The continuous nature of the helical sensor array enables detection of grip pressure waves that propagate along the grip during swing acceleration and impact, providing insights into energy transfer efficiency and swing mechanics optimization.

The electrical routing architecture for helical films may employ a streamlined approach that takes advantage of the continuous substrate geometry to minimize connection complexity and maximize signal integrity. The primary routing traces may follow the helical path of the film, creating natural pathways for signal transmission from distributed sensor elements to centralized connection points. Secondary routing traces may be oriented perpendicular to the helical direction to create grid-like interconnection patterns that enable individual addressing of sensor elements while maintaining the structural helical design.

The helical routing system may converge at connection points positioned at the handle termination regions, where the film ends can be connected to internal processing electronics or external monitoring systems. The continuous nature of the helical film enables the use of fewer connection points compared to segmented approaches, reducing the mechanical complexity and potential failure modes associated with multiple interconnections. The helical geometry also provides natural strain relief for electrical connections, as the spiral configuration distributes mechanical stress over extended lengths rather than concentrating stress at discrete connection points.

The helical film may incorporate the same multi-modal impedimetric sensing capabilities described for spherical applications, including pressure measurement, temperature sensing, humidity detection, and vibration analysis, but instead may be used to assess cylindrical and/or handle-based sporting equipment. The continuous helical coverage enables comprehensive thermal mapping of grip temperature distribution, which may be used to analyze grip comfort and performance under varying environmental conditions and extended use periods.

The helical sensor array may assess and/or detect dynamic grip events such as grip pressure waves during impact, grip rotation during stroke execution, and grip stability variations during rapid movement sequences. The continuous sensing coverage eliminates dead zones that could occur with segmented approaches, ensuring that all grip interactions are captured regardless of hand position, grip style, or equipment orientation. This comprehensive sensing capability supports advanced performance analysis applications including technique optimization, equipment fitting, and injury prevention research for various sporting applications.

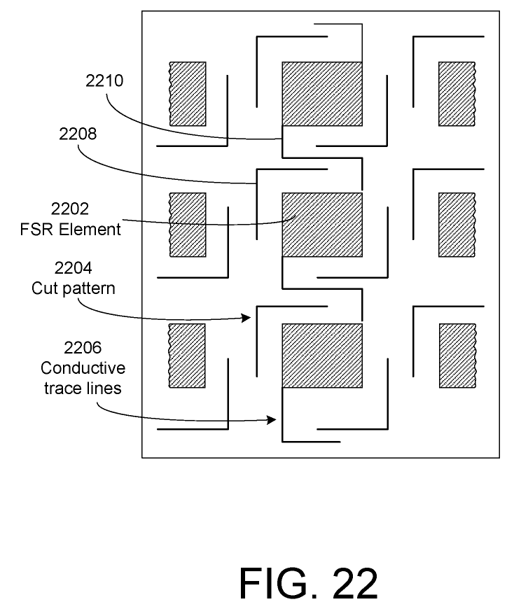

FIG. 22 illustrates an example sensor foil design for the sensor arrays described herein. In some embodiments, the top layer of the force sensitive resistor (FSR) array may be patterned with any number of slits or cuts that may reduce the force utilized to deflect the top FSR elements into contact with the bottom FSR elements. The cut pattern may create any number of discrete FSR sensing regions (e.g., islands) connected to one another by a plurality of structural regions, referred to herein as bridges. The islands and bridges collectively form a patterned substrate architecture that improves the sensitivity of the force-conductivity curve by reducing the local bending stiffness of the thin-film substrate at predetermined locations. The reduction in local bending stiffness may allow the top layer of the FSR array to deflect under applied force, lowering the activation threshold required to establish electrical contact between the top and bottom FSR elements. Additionally, the island and bridge architecture may improve a spatial resolution of the FSR array by isolating the deflection area of the thin-film substrate to the region immediately surrounding each individual FSR island, thereby reducing mechanical coupling between adjacent sensor elements and enabling more precise localization of applied forces across the array.

In some embodiments, a number of conductive electrical trace lines may be routed across the bridge regions of the patterned substrate to electrically interconnect rows or columns of FSR elements within the array. The conductive electrical trace lines may follow a routing path that traverses the bridge regions between adjacent FSR islands, providing continuous electrical connectivity across the array while accommodating the mechanical deformation of the bridge regions under applied force. The routing path of the conductive electrical trace lines across the bridge regions may be configured to minimize mechanical stress on the conductive pathways during deflection of the top layer, maintaining signal integrity throughout the sensing range of the FSR array.

In some embodiments, the bottom thin-film printed circuit layer of the FSR array may be coupled to a semi-rigid surface, which may be flat or curved, such that the bottom layer remains substantially fixed during operation. The sensing operation of the FSR array may be based on deformation of the top layer toward the fixed bottom layer in response to applied force. The top layer may deflect locally at each FSR island in response to applied pressure, establishing electrical contact between the top and bottom FSR elements at the location of the applied force while the bottom layer remains substantially stationary against the semi-rigid surface. This configuration may enable consistent and repeatable sensing performance across the FSR array when applied to surfaces of varying curvature, including surfaces having non-zero Gaussian curvature.

The sensor foil design 2200 may include one or more of a plurality of FSR (force sensitive resistor) elements 2202, a cut pattern 2204, and conductive trace lines 2206. The sensor foil design 2200 may be representative of a portion of the thin-film substrate described herein, illustrating the spatial relationship between the sensing elements, the strain-relief cut patterns, and the electrical routing architecture as they would appear in a planar, pre-conformed state prior to application to a curved surface having non-zero Gaussian curvature.

The FSR elements 2202 may represent individual force-sensing resistor elements disposed on a thin-film substrate. Each FSR element 2202 may include a central region filled with diagonal hatching, indicating the active sensing area of the respective sensor element. The FSR elements 2202 may be the same as or similar to the sensor elements 312, 404, 620, 706, 1704, and/or 1806, as described elsewhere herein. In some embodiments, the FSR elements 2202 may be arranged in a non-Cartesian tessellated pattern across the thin-film substrate to maintain substantially uniform spatial sensing resolution when the substrate is conformed to a curved surface. The FSR elements 2202 may be distributed in a loose grid pattern across the substrate, with each element occupying a discrete island region of the substrate that is mechanically isolated from adjacent elements by the surrounding cut pattern 2204. This island configuration may allow each FSR element 2202 to maintain dimensional stability and consistent contact area during deformation of the thin-film substrate, which is particularly important for pressure-sensitive applications where the overlap area between the top and bottom foil layers directly determines measurement accuracy.

In some embodiments, the FSR elements 2202 may be printed on the thin-film substrate using carbon-loaded polymer ink with a resistance range of about 500 ohms to about 10 kiloohms, as described with respect to the manufacturing process 2100 of FIG. 21. The active sensing area of each FSR element 2202 may be defined by the overlap region between a top foil layer and a bottom foil layer of the multilayer substrate construction, as described with respect to FIGS. 17 and 18. The diagonal hatching pattern visible within each FSR element 2202 in FIG. 22 may correspond to this active overlap region, where applied pressure causes a measurable change in electrical resistance that is transmitted through the conductive trace lines 2206 to the integrated circuit for analysis.

The cut pattern 2204 may represent the strategically positioned cutouts and openings within the thin-film substrate that surround each FSR element 2202. The cut pattern 2204 may include square or rectangular outlines that define the perimeter of each discrete island region containing an FSR element 2202. In some embodiments, the cut pattern 2204 may be the same as or similar to the patterned cutouts 306, 412, 604, and/or holes 2004, as described elsewhere herein. The cut pattern 2204 may be formed using laser cutting or die cutting techniques applied to the thin-film substrate after deposition of the conductive trace lines and sensor elements, as described with respect to the manufacturing process 2100 of FIG. 21.

The cut pattern 2204 may function as a strain relief mechanism that enables localized deformation of the thin-film substrate when the substrate is conformed to a curved surface having non-zero Gaussian curvature. By removing substrate material in the regions surrounding each FSR element 2202, the cut pattern 2204 creates controlled deformation points that allow the substrate to accommodate the compound curvature of the target surface without generating excessive mechanical stress at the sensor element locations. The rectangular or square outline geometry of the cut pattern 2204 visible in FIG. 22 may be selected to provide substantially uniform strain relief in multiple directions, enabling the substrate to conform to surfaces with curvature in more than one plane simultaneously. In some embodiments, the depth and spacing of the cut pattern 2204 may be selected based on the diameter or radius of curvature of the target surface to minimize buckling deformation of the thin-film substrate and to minimize signal disruption between the integrated circuit and the FSR elements 2202 when the substrate is curved about the surface and/or minimize sensor performance degradation of one or more of the sensor elements.

The cut pattern 2204 may further define the boundaries between adjacent FSR elements 2202, creating a tessellated arrangement of discrete sensing islands interconnected by the conductive trace lines 2206. The open regions created by the cut pattern 2204 between adjacent FSR elements 2202 may allow the substrate to flex and deform locally in response to the curvature of the target surface, while the intact substrate material within each island region maintains the structural integrity and dimensional stability of the FSR element 2202 disposed thereon. In some embodiments, the cut pattern 2204 may include additional internal cutouts or auxetic slit patterns within the island regions to provide further mechanical compliance without compromising the sensing functionality of the FSR elements 2202.

The conductive trace lines 2206 may represent the electrical routing pathways that interconnect the FSR elements 2202 and transmit signals from the sensor array to the centralized routing hub or ring bus described elsewhere herein. The conductive trace lines 2206 may be visible in FIG. 22 as pairs of traces (e.g., left partial square-shaped trace 2208 and right hysteresis-shaped trace 2210) at connection points or junctions. The traces 2208, 2210 may correspond to different routing layers or different signal paths within the multilayer electrical routing architecture, such as row lines and column lines of a sensor matrix addressing scheme, or top foil traces and bottom foil traces of the dual-foil construction described with respect to FIGS. 17 and 18.

The conductive trace lines 2206 (traces 2208, 2210, etc.) may be arranged to follow paths of minimal curvature along the thin-film substrate, consistent with the hierarchical tree-like routing architecture described elsewhere herein. In some embodiments, the conductive trace lines described herein may extend along the central meridian of each petal-shaped segment of the thin-film substrate, with branch lines extending laterally to connect individual FSR elements 2202 within each segment. The L-shaped and U-shaped routing paths visible in the conductive trace lines 2206 of FIG. 22 may correspond to the branch routing lines that extend outward from a central trunk line to individually address each FSR element 2202, as described with respect to the hierarchical routing architecture of the grip assessment systems herein.

Segments 2210 in the conductive trace lines 2206 may represent continuous routing paths that extend from one FSR element 2202 to another, forming a connected network of sensing elements across the substrate. These segments 2210 may correspond to the column routing lines of the sensor matrix, which connect multiple FSR elements 2202 in a vertical direction across the substrate. The segments 2208 in trace lines 2206 may represent the row routing lines that connect FSR elements 2202 in a horizontal direction, forming the orthogonal addressing structure of the sensor matrix. The combination of row and column routing lines enables individual addressing of each FSR element 2202 through multiplexing at the centralized routing hub, as described with respect to the ring bus 505 and central hub 308 of FIGS. 3 and 5.

In some embodiments, the conductive trace lines 2206 may be screen-printed onto the thin-film substrate using silver-based conductive ink with line widths of about 100 microns to about 250 microns and a thickness of about 8 microns to about 15 microns, as described with respect to the manufacturing process 2100 of FIG. 21. Dielectric insulating ink bridges may be applied at junctions where the conductive trace lines 2206 intersect or cross, preventing electrical shorts between the row and column routing lines while maintaining signal integrity across the sensor array. The dielectric insulating ink bridges may have dimensions of about 1.5 millimeters by about 1.0 millimeters with a thickness of about 20 microns to about 30 microns, as described with respect to the sensor cross-section 1700 of FIG. 17.

The overall arrangement of FSR elements 2202, cut pattern 2204, and conductive trace lines 2206 visible in FIG. 22 may illustrate the non-Cartesian tessellated layout of the sensor array in its planar, pre-conformed state. The alternating orientation of adjacent FSR elements 2202 and the interconnected routing paths of the conductive trace lines 2206 may provide substantially uniform spatial sensing resolution across the substrate, which translates to uniform sensing coverage when the substrate is conformed to a curved surface having non-zero Gaussian curvature. The sensor foil design 2200 may be applied to the petal-shaped segments of the thin-film substrate described elsewhere herein, with the cut pattern 2204 providing the strain relief necessary for the petal-shaped segments to deform predictably when applied to the curved surface and the conductive trace lines 2206 routing signals from the FSR elements 2202 along the central meridian of each segment toward the centralized routing hub or ring bus.

The island regions visible in FIG. 22 may correspond to the discrete FSR sensing regions described herein, with each island region comprising an individual FSR element 2202 mechanically isolated from adjacent island regions by the surrounding cut pattern 2204. The bridge regions connecting adjacent island regions may correspond to the structural regions described herein that provide mechanical continuity between adjacent FSR islands while allowing localized deflection of each island toward the bottom FSR layer in response to applied force. The conductive trace lines 2206 visible traversing the bridge regions between adjacent FSR islands may correspond to the electrical routing pathways described herein that interconnect rows and columns of FSR elements while accommodating the mechanical deformation of the bridge regions during sensing operation

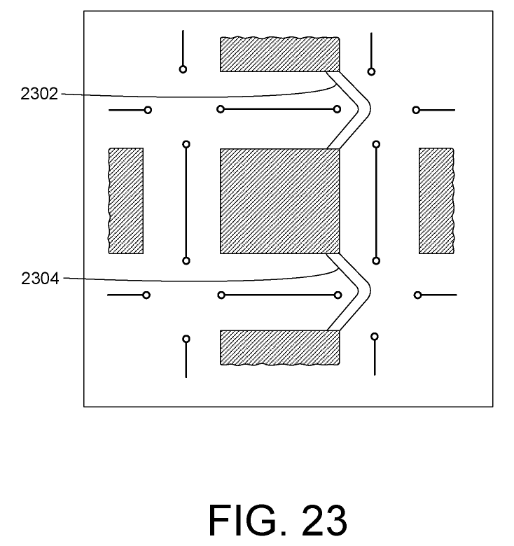

FIG. 23 illustrates an example sensor foil design 2300 for the sensor arrays described herein. In some embodiments, the patterned cuts in the top layer of the FSR array may be arranged in a four corner hinge configuration in which a number of cuts extend inward from positions adjacent to each corner of a respective FSR island. The four corner hinge configuration may define a discrete region of thin-film substrate material at each corner of the FSR island that provides mechanical support for the FSR pad while enabling localized deflection of the island toward the bottom FSR layer in response to applied force. The width of the supporting material at each corner may be determined by the length of the cuts extending inward from the adjacent positions, such that longer cuts produce narrower corner support regions and lower activation force thresholds, while shorter cuts produce wider corner support regions and higher activation force thresholds. The cut length may therefore be used as a design parameter to tune the force-conductivity response of individual FSR elements within the array, enabling customization of sensitivity characteristics across different regions of the sensor array. The symmetric distribution of corner support material may cause the FSR island to deflect substantially uniformly toward the bottom FSR element when force is applied at or near the center of the island, resulting in a substantially uniform contact area between the top and bottom FSR elements and a consistent force-conductivity response.

In some embodiments, the four corner hinge configuration may improve the spatial resolution of the FSR array by mechanically isolating the deflection of each FSR island from adjacent islands, reducing mechanical coupling between neighboring FSR elements and confining the deflection response substantially to the island on which force is applied. The conductive electrical trace lines coupling each FSR island to the routing architecture of the sensor array may be routed across the bridge regions between adjacent islands rather than across the cut regions of the four corner hinge configuration, preserving the mechanical compliance of the corner hinge regions by avoiding additional conductive material that could increase local bending stiffness and raise the activation force threshold. In some embodiments, the four corner hinge configuration may be applied to FSR arrays disposed on thin-film substrates configured to conform to surfaces having non-zero Gaussian curvature, with cut lengths selected based on the radius of curvature of the target surface to compensate for increased mechanical stiffness introduced during the conforming process, thereby maintaining consistent activation force thresholds and sensitivity characteristics after application to the curved surface.

As shown in FIG. 23, the sensor foil design 2300 may include one or more of a first routing region 2302 and a second routing region 2304. The sensor foil design 2300 may represent a portion of the thin-film substrate described herein, illustrating an alternative routing architecture for the conductive electrical trace lines as they would appear in a planar, pre-conformed state prior to application to a curved surface having non-zero Gaussian curvature. The sensor foil design 2300 may be understood as a continuation or variation of the sensor foil design 2000 of FIG. 22, depicting a different region of the petal-shaped segment or an alternative trace routing configuration for connecting FSR elements to the centralized routing hub or ring bus.

The first routing region 2302 may represent an upper routing pathway within the sensor foil design 2300. The first routing region 2302 may include a green angled or chevron-shaped conductive trace path that bends at a predetermined angle and connects to a horizontal black line segment extending toward the left side of the substrate. The angled geometry of the first routing region 2302 may correspond to a branch routing line that extends outward from a central trunk routing line to individually address one or more FSR elements within the petal-shaped segment, consistent with the hierarchical tree-like routing architecture described elsewhere herein. The horizontal line segments associated with the first routing region 2302 may represent row routing lines that connect FSR elements in a horizontal direction across the substrate, forming part of the orthogonal addressing structure of the sensor matrix. The small circular endpoints visible at the terminations of the horizontal line segments may correspond to electrical connection points or through-hole vias that establish vertical electrical connections between non-adjacent conductive layers of the multilayer substrate construction, as described with respect to the grip assessment systems herein.

The second routing region 2304 may represent a lower routing pathway within the sensor foil design 2300. The second routing region 2304 may include a green angled or chevron-shaped conductive trace path substantially mirroring the geometry of the first routing region 2302, connecting to a corresponding horizontal black line segment extending toward the left side of the substrate. The second routing region 2304 may correspond to a second branch routing line that addresses a separate row or column of FSR elements within the petal-shaped segment. The vertically mirrored or repeated structure formed by the first routing region 2302 and the second routing region 2304 may illustrate the repeating unit cell of the hierarchical routing architecture, in which pairs of branch routing lines extend symmetrically from the central trunk to address adjacent rows of FSR elements distributed across the petal-shaped segment.

The diagonal hatching visible within the rectangular regions associated with the first routing region 2302 and the second routing region 2304 may correspond to the active sensing areas of the FSR elements disposed on the thin-film substrate, consistent with the FSR elements 2202 described with respect to FIG. 22. The red line segments visible along certain edges of the rectangular regions may correspond to the cut pattern 2204 described with respect to FIG. 22, indicating the boundaries of the discrete island regions that surround each FSR element and provide strain relief during conforming of the substrate to a curved surface having non-zero Gaussian curvature.

The wavy outer boundary of the sensor foil design 2300 visible in FIG. 23 may correspond to the irregular perimeter of the thin-film substrate after the cut pattern has been applied, consistent with the sensor foil design 2000 of FIG. 20. The removal of excess substrate material in non-active areas reduces surface buckling and improves conformability to the target curved surface, as described with respect to the manufacturing process 2100 of FIG. 21. The overall arrangement of routing regions 2302 and 2304 within the sensor foil design 2300 may illustrate how the hierarchical tree-like routing architecture accommodates the geometric constraints of the petal-shaped segment while maintaining electrical connectivity between the distributed FSR elements and the centralized routing hub or ring bus.

In some embodiments, the angled routing geometry depicted in the first routing region 2302 and the second routing region 2304 may be selected to minimize the total length of conductive trace material while maintaining signal integrity across the sensor array. The chevron or angled path geometry may provide natural strain relief for the conductive trace lines when the thin-film substrate is deformed about the bridge regions and conformed to the curved surface, as the angled segments can accommodate minor dimensional changes without generating excessive mechanical stress at the trace line locations. This routing geometry may be particularly advantageous in regions of the petal-shaped segment that experience significant curvature during the conforming process, such as near the distal end of each protrusion where the substrate converges at the apex of the hemispherical shell.

In some embodiments, the conductive trace lines depicted in the first routing region 2302 and the second routing region 2304 may be screen-printed onto the thin-film substrate using silver-based conductive ink with line widths of about 100 microns to about 250 microns and thickness of about 8 microns to about 15 microns, as described with respect to the manufacturing process 2100 of FIG. 21. Dielectric insulating ink bridges may be applied at junctions where the routing lines of the first routing region 2302 and the second routing region 2304 intersect or cross other conductive pathways, preventing electrical shorts between different signal paths while maintaining signal integrity across the sensor array. The dielectric insulating ink bridges may have dimensions of about 1.5 millimeters by about 1.0 millimeters with thickness of about 20 microns to about 30 microns, as described with respect to the sensor cross-section 1700 of FIG. 17.

The example cut pattern visible in FIG. 23 may correspond to the four corner hinge configuration described herein, with cuts extending inward from positions adjacent to each corner of the respective FSR island to define corner support regions at each corner of the island. The width of the corner support regions visible in FIG. 23 may correspond to the width of supporting material determined by the length of the cuts extending inward from each corner position, as described herein. The hatched central regions visible within each FSR island in FIG. 23 may correspond to the active sensing areas of the respective FSR elements, while the surrounding cut regions define the mechanical compliance of the four corner hinge configuration.

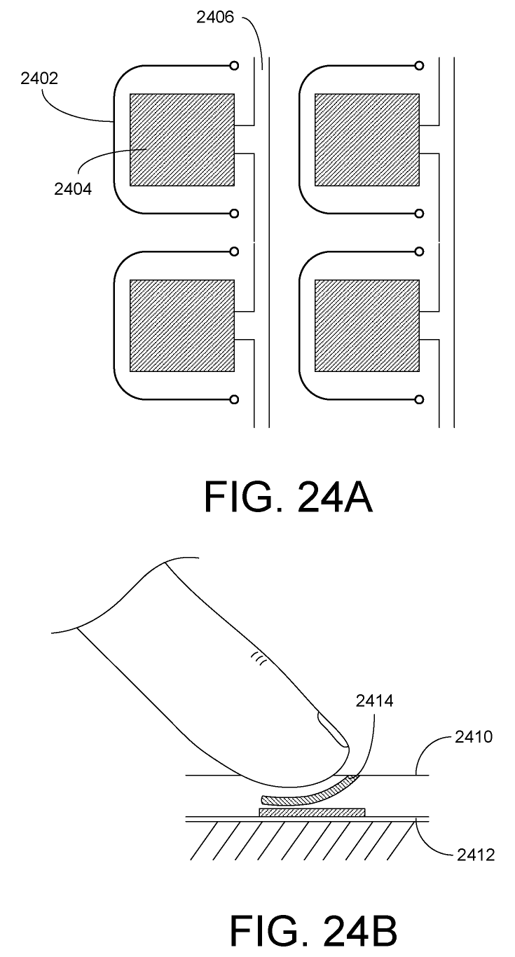

FIG. 24A-24C illustrate example pattern cutouts for the sensor foils described herein. FIG. 24A illustrates a first sensor element configuration 2400 showing a 2 by 2 arrangement of sensor element units on a thin-film substrate. Each element unit may include a rounded rectangular outline 2402 defining a perimeter of an individual sensor element, with a central hatched region representing the active sensing area 2404 of the respective FSR element. The rounded rectangular geometry 2402 of each element unit may be selected to minimize stress concentration at the corners of the sensor element during deformation of the thin-film substrate when conformed to a curved surface having non-zero Gaussian curvature. A central routing line 2406 extends vertically between the two columns of element units and may correspond to the central trunk routing line of the hierarchical tree-like routing architecture described elsewhere herein, with horizontal branch lines extending from the trunk to connect to each individual element.

FIG. 24B illustrates a cross-sectional side view of a sensor element operation mechanism showing the interaction between a finger, palm, or other body portion, a top FSR layer 2410, a bottom FSR layer 2412, and a fixed surface during a grip interaction. As depicted in FIG. 24B, when a finger (or other body part) applies pressure to the surface of the conformal sensor device, the top FSR layer 2410 undergoes local deformation in the region directly beneath the body part contact area. The local deformation of the top FSR layer 2410 causes the active sensing area of the top FSR layer to deflect toward the bottom FSR layer 2412, creating an FSR contact area at the interface between the two layers 2410, 2412. The FSR contact area represents the overlap region between the top and bottom FSR layers where applied pressure causes a measurable change in electrical resistance that is transmitted through the conductive trace lines to the integrated circuit for analysis.

A hinge flap feature 2414 may correspond to the patterned cutouts and edge slits described elsewhere herein that facilitate local strain deformation and reduce buckling when the thin-film substrate is adhered to high-curvature regions of the target surface. The hinge flap 2414 may function as a flexible connection point that allows the top FSR layer to deflect locally under applied pressure while maintaining structural continuity with the surrounding substrate material. The fixed surface beneath the bottom FSR layer may correspond to the hemispherical shell 702 described with respect to FIGS. 7-11, onto which the thin-film substrate is adhered using the adhesive backing layer described elsewhere herein.

In some embodiments, the local deformation mechanism depicted in FIG. 24B may be enhanced by the cut pattern 2204 described with respect to FIG. 22, which creates discrete island regions that allow each FSR element to deflect independently under applied pressure without transmitting mechanical stress to adjacent sensor elements. This island configuration may improve the spatial resolution of the sensor array by isolating the deflection area of the top FSR layer to the region directly beneath each individual finger contact, for example, enabling the grip assessment systems described herein to distinguish between adjacent finger contacts and accurately map the pressure distribution across the curved surface.

The sensitivity of the force-conductivity response of each FSR element may be improved by reducing the local bending stiffness of the thin-film substrate in the regions surrounding each sensor element through the application of the cut pattern. By creating FSR islands connected by bridges of substrate material, the cut pattern reduces the force utilized to deflect the top FSR layer 2410 into contact with the bottom FSR layer 2412, resulting in improved pressure sensitivity performance across the sensor array. In some embodiments, the pressure sensitivity performance may be about 0.5 to about 5% resistance change per 100 grams of applied force, as described with respect to the manufacturing process 2100 of FIG. 21.

FIG. 24C illustrates a second sensor element configuration 2400C showing an alternative 2 by 2 arrangement of sensor element units with a modified central routing architecture. The example pattern shown in FIG. 24C depicts an orthogonal C pattern with cutouts rotated 90 degrees with respect to neighboring cutouts/elements to further isolate local bending.

The example element units (e.g., element unit 2420) may be arranged in a symmetric pattern around a central vertical axis, with each unit containing a centrally located hatched region 2422 representing the active sensing area of the respective FSR element. The routing connections 2424 may correspond to alternative routing connection geometries that provide electrical connectivity between adjacent element units while accommodating the mechanical constraints imposed by the surrounding cut pattern.

In some embodiments, the sensor element configurations depicted in FIGS. 24A and 24C may be selected based on the specific curvature characteristics of the target surface and the desired spatial sensing resolution of the conformal sensor device. The rounded rectangular element geometry of FIG. 24A may be preferred for applications utilizing uniform pressure sensitivity across the entire active sensing area of each FSR element, while the alternative configuration of FIG. 24C may be preferred for applications requiring enhanced routing flexibility or improved mechanical compliance in regions of high curvature. Both configurations may be manufactured using the screen-printing and curing processes described with respect to the manufacturing process 2100 of FIG. 21, with modifications to the cut pattern geometry and routing architecture to accommodate the specific element configuration selected.

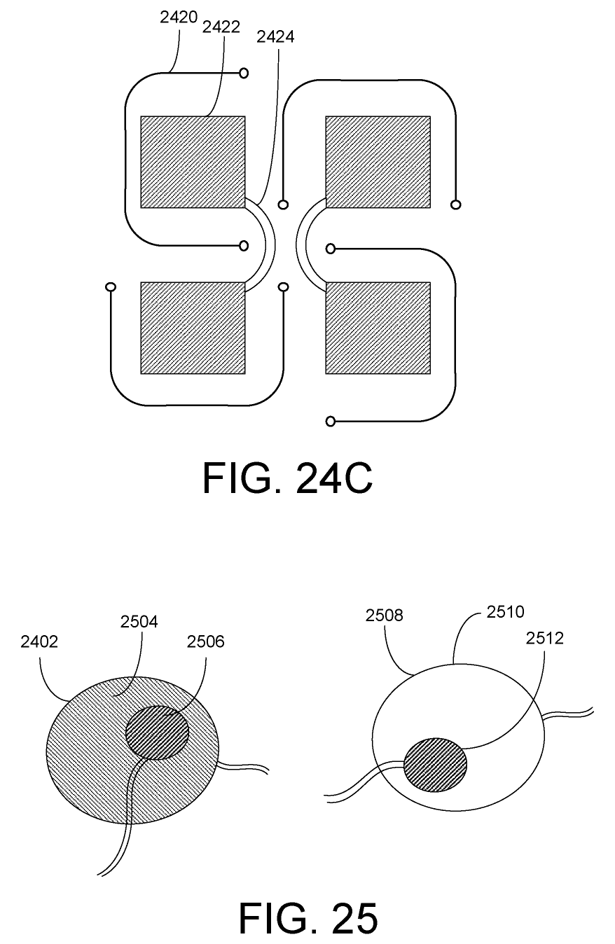

FIG. 25 illustrates an example pad shape for force sensitive resistor sensors described herein. In some embodiments, the top and bottom thin-film substrates of a through mode FSR array may experience shifting and rotation relative to each other during manufacturing or during application to a curved surface having non-zero Gaussian curvature, such as a sphere or ellipsoid, resulting in non-uniform overlap area between opposing FSR elements that may produce inconsistent sensitivity characteristics and reduced measurement accuracy across the sensing surface. To address this issue, the top and bottom FSR pads may be printed with different sizes such that one pad remains completely contained within the boundary of the other pad under small translations or rotations of the substrate layers relative to each other, thereby maintaining a substantially uniform and consistent overlap area throughout manufacturing and after application to the curved surface. In one non-limiting example, the top and bottom FSR pads may be printed as concentric circles in which the diameter of the bottom FSR element is larger than the diameter of the top FSR element by a predetermined offset distance selected to accommodate the maximum expected relative displacement between the substrate layers, ensuring that the effective contact area between the top and bottom FSR elements remains substantially equal to the area of the smaller top FSR element regardless of small misalignments, with the predetermined offset distance selected based on the radius of curvature of the target surface to account for larger relative substrate displacements anticipated during conforming to surfaces of greater curvature.

As shown in FIG. 25, the sensor pad geometries may include a first pad 2502 configuration showing a bottom pad 2504 and a top pad 2506 arranged concentrically, and a second pad 2508 configuration showing a bottom pad 2510 and a top pad 2512 arranged with an offset relationship. The bottom pad 2502, 2508 in each configuration may correspond to the sensor element disposed on the bottom foil layer of the multilayer substrate construction described with respect to FIGS. 17 and 18, while the top pad 2506, 2512 may correspond to the sensor element disposed on the top foil layer. The overlap area between the top pad and the bottom pad in each configuration may define the active sensing area of the respective FSR element, where applied pressure causes a measurable change in electrical resistance.

In some embodiments, the pad geometries depicted in FIG. 25 may be selected based on the specific application requirements of the conformal sensor device. Circular pad geometries may be preferred for applications requiring isotropic pressure sensitivity, as the circular shape provides equal sensitivity to applied forces from any direction within the plane of the sensor element. The offset pad configuration may be particularly advantageous for applications involving significant substrate deformation during conforming, as the maintained overlap area ensures consistent sensor performance despite the geometric distortions that may occur when the thin-film substrate is applied to surfaces with high Gaussian curvature.

In some embodiments, the pad geometries depicted in FIG. 25 may be manufactured using the screen-printing and curing processes described with respect to the manufacturing process 2100 of FIG. 21, with the carbon-loaded polymer ink deposited in the circular or curved pad shapes defined by the respective pad configurations. The dielectric insulating ink bridges described with respect to the manufacturing process 2100 may be applied at junctions where the conductive trace lines connecting to the top and bottom pads intersect or cross, preventing electrical shorts between the row and column routing lines while maintaining signal integrity across the sensor array. The adhesive layer described with respect to the multi-foil architecture may maintain alignment between the top and bottom pad layers during deformation of the thin-film substrate, ensuring that the overlap area between the top pad and the bottom pad remains substantially constant throughout the conforming process and during operational use of the conformal sensor device.

In some embodiments, the sensor pad geometries described herein may be combined with the non-cartesian tessellated arrangements described elsewhere herein to provide sensor arrays with both uniform spatial sensing resolution and consistent pressure sensitivity performance across curved surfaces having non-zero Gaussian curvature. The circular or curved pad geometries may be arranged in hexagonal, triangular, or other non-rectangular tessellated patterns across the petal-shaped segments of the thin-film substrate, with the cut pattern positioned between adjacent pad regions to provide strain relief during conforming. The conductive trace lines connecting the pad regions may follow the hierarchical tree-like routing architecture described elsewhere herein, with trunk lines extending along geodesic paths of minimal curvature and branch lines extending to individually address each pad region within the tessellated arrangement.

In some embodiments, the conformal sensor devices and grip assessment systems described herein may be calibrated prior to use to account for variations in sensor element performance that may arise from the conforming process. The calibration process may include applying known reference pressures to the sensor array after conforming to the curved surface and recording the corresponding resistance changes from each FSR element. The recorded calibration data may be stored in the memory 256 of the monitoring system 200 and applied by the calibration routines 224 of the grip assessment engine 220 to correct for sensor-to-sensor variations and substrate deformation effects during subsequent grip assessment operations. The calibration process may be repeated periodically to account for drift in sensor performance over time or after significant mechanical events such as impacts or drops.

In some embodiments, the conformal sensor devices described herein may incorporate temperature compensation capabilities to account for the temperature dependence of the resistive sensing elements. The temperature sensing capabilities described elsewhere herein may be used to measure the local temperature at each sensor element location, and the temperature measurements may be applied by the signal processing module 104 to correct the pressure measurements for temperature-induced resistance changes. This temperature compensation may be particularly important for outdoor sports applications where the sensor device may be exposed to significant temperature variations during use, and for medical device applications where body temperature variations may affect sensor performance.

In some embodiments, the conformal sensor devices and grip assessment systems described herein may be integrated with inertial measurement units (IMUs) or other motion sensing devices to provide comprehensive characterization of both grip interactions and device motion. The IMU data may be combined with the pressure and grip data from the sensor array by the analysis module 222 to provide insights into the relationship between grip characteristics and device motion patterns, enabling advanced performance analysis applications such as pitch type classification for baseball, swing mechanics analysis for golf, or stroke technique assessment for tennis. The combined sensor data may be processed by the ML models 252 to identify correlations between grip patterns and motion outcomes, providing actionable feedback for technique improvement and performance optimization.

In some embodiments, the conformal sensor devices described herein may be manufactured with biocompatible materials for medical device applications requiring direct skin contact or implantable configurations. The thin-film substrate materials may be selected from biocompatible polymers such as medical-grade polyimide or parylene-coated substrates, and the conductive trace lines and sensor elements may be manufactured using biocompatible conductive materials such as gold, platinum, or biocompatible conductive polymers. The adhesive layer may be formulated using medical-grade pressure-sensitive adhesives that provide secure bonding to biological surfaces while maintaining biocompatibility and skin compatibility for extended wear applications.

In some embodiments, the conformal sensor devices and grip assessment systems described herein may be manufactured using roll-to-roll printing processes to enable high-volume production of the thin-film sensor arrays. The roll-to-roll process may include continuous deposition of conductive electrical trace lines, dielectric insulating ink, and sensor elements onto a flexible substrate film using rotary screen printing or gravure printing techniques, followed by continuous curing using infrared or UV radiation sources. The patterned cutouts and edge slits may be formed using rotary die cutting or laser cutting systems integrated into the roll-to-roll production line, enabling continuous high-speed manufacturing of the petal-shaped segments with precise cut pattern geometries. The roll-to-roll manufacturing approach may reduce production costs and improve manufacturing consistency compared to sheet-fed printing processes, enabling cost-effective production of conformal sensor devices for high-volume consumer applications such as sports equipment and wearable electronics.

In some embodiments, the conformal sensor devices described herein may incorporate wireless power transfer capabilities to enable battery-free operation in applications where battery replacement or charging is impractical. The wireless power transfer system may include an inductive coil integrated into the thin-film substrate or the printed circuit board 502, configured to receive power from an external wireless charging source through electromagnetic induction. The received power may be conditioned by power management circuitry on the printed circuit board 502 and distributed to the sensor array, signal processing electronics, and wireless communication module. The wireless power transfer capability may enable continuous operation of the conformal sensor device without the need for battery replacement or wired charging connections, which may be particularly advantageous for applications involving sealed or waterproof enclosures such as sports balls or medical implants.

In some embodiments, the conformal sensor devices and grip assessment systems described herein may be configured for multi-user operation, enabling simultaneous monitoring of grip interactions from multiple users interacting with the same or different sensor devices. The communication engine 240 may support multi-device communication protocols that enable data from multiple sensor devices to be transmitted to a central monitoring system for comparative analysis. The ML models 252 may be trained to identify individual users based on their unique grip signatures, enabling the monitoring system 200 to automatically associate grip data with specific user profiles 214 without requiring manual user identification. This multi-user capability may be particularly valuable for team sports applications, coaching environments, or clinical settings where multiple users interact with sensor devices during the same monitoring session.

In some embodiments, the conformal sensor devices described herein may be configured for use with augmented reality (AR) or virtual reality (VR) systems to provide haptic feedback and grip interaction data for immersive simulation applications. The grip pressure and finger placement data from the sensor array may be transmitted in real-time to the AR or VR system through the communication interface 110, enabling the simulation environment to respond dynamically to the user's grip interactions and thereby simulate various results from the user interactions. The ML models 252 may be trained on grip interaction datasets from specific simulation scenarios to also provide predictive grip guidance and technique coaching within the AR or VR environment. This integration may enable advanced training applications for sports, surgery, or other skill-based activities that require precise grip control and technique development.

In some embodiments, the conformal sensor devices and grip assessment systems described herein may be configured for long-term monitoring applications requiring extended operational lifetimes of months or years. The thin-film substrate materials and printed electronics may be selected and manufactured to provide long-term stability under repeated mechanical deformation, temperature cycling, and exposure to moisture or other environmental factors. Encapsulation layers may be applied over the sensor array and conductive trace lines to protect the printed electronics from environmental degradation while maintaining the mechanical flexibility and conformability of the thin-film substrate. The power management system may be optimized for low-power operation to extend battery life for long-term monitoring applications, with the sensor array operating in a duty-cycled mode that balances sensing frequency with power consumption based on the specific monitoring requirements of the application.

As used herein, the term ‘conform smoothly’ may refer to the condition achieved when an inelastic thin-film substrate is applied to a surface having curvature such that the substrate maintains substantially continuous and intimate contact with an underlying surface across the full extent of each segment. A thin-film substrate that conforms smoothly to a curved surface does not exhibit excessive wrinkling, buckling, folding, or material buildup of a degree that would create measurable gaps between the substrate and the surface, cause misalignment of sensor elements relative to their intended positions, introduce mechanical stress concentrations sufficient to cause tearing or delamination of the substrate layers, or produce signal noise or measurement artifacts in the output of the sensor array. It should be understood that smooth conformity as used herein does not necessarily indicate a complete absence of all substrate deformation, as localized deformation at patterned cutout regions and controlled overlapping of adjacent petal-shaped segments at boundaries are expected and intended features of the conformal application process. Rather, smooth conformity may infer that any such deformation or overlap does not materially degrade the spatial sensing resolution, electrical integrity, or signal quality of the sensor array disposed on the thin-film substrate. The degree of smooth conformity achievable for a given thin-film substrate may depend on factors including a radius of curvature of the target surface, a geometry and depth of the patterned cutouts, a number and width of shaped segments, and/or mechanical properties of the substrate material, all of which may be selected and optimized to achieve smooth conformity for a particular curved surface application.

As used herein, the patterning of the sensor array and the patterning of the thin-film substrate upon which the sensor array is disposed are distinct concepts that are described using distinct terminology throughout this disclosure. The term "sensor element geometry" refers to the two-dimensional shape of each individual sensor element as printed on the thin-film substrate, which may include non-rectangular shapes such as triangular, hexagonal, oval, or polygonal configurations selected to maximize sensing area coverage and minimize dead zones when the substrate is conformed to a curved surface. The term "sensor element spatial distribution" refers to the arrangement of sensor elements relative to one another across the surface of the thin-film substrate, which may follow non-Cartesian patterns such as tessellated, Voronoi, fractal-like, or asymmetric density distributions selected to maintain substantially uniform spatial sensing resolution when the substrate is deformed to fit a substantially curved surface.

As used herein, the term ‘substrate cut pattern’ may refer to a geometric configuration of slits, cutouts, holes, and openings formed in the thin-film substrate to enable localized mechanical deformation and strain relief during application to a curved surface having non-zero Gaussian curvature. The substrate cut pattern may include auxetic structures, wave-shaped or sawtooth-shaped edge cutouts, four corner hinge configurations, C-pattern cutouts, orthogonal C-pattern cutouts, and island-and-bridge architectures, each of which creates controlled deformation points in the substrate that allow the substantially inelastic thin-film material to conform smoothly to non-developable surfaces without excessive wrinkling, buckling, or tearing. The term ‘substrate segment architecture’ may refer to the overall geometric layout of the thin-film substrate including one or more of a central hub, bridge regions, and shaped segments that collectively define a macroscopic shape of the substrate in its undeformed planar state prior to application to the curved surface.

As used herein, the term 'substantially curved surface' may refer to any surface having non-zero Gaussian curvature that cannot be flattened onto a plane without distortion, including but not limited to spherical surfaces, ellipsoidal surfaces, prolate spheroid surfaces, hyperbolic paraboloid surfaces, and other non-developable surfaces. A substantially curved surface as used herein does not include purely cylindrical surfaces, which have zero Gaussian curvature, unless the cylindrical surface is combined with or transitions into a surface region having non-zero Gaussian curvature.

As used herein, the term 'non-cartesian pattern' may refer to a spatial arrangement of sensor elements on the thin-film substrate that does not follow a rectangular grid layout defined by two mutually perpendicular axes. A non-cartesian pattern may include tessellated arrangements such as hexagonal, triangular, Voronoi, or fractal-like distributions, asymmetric density distributions, radial arrangements following meridian lines of a curved surface, or any other spatial distribution of sensor elements that departs from a conventional rectangular row-and-column grid layout. A sensor array disposed in a non-cartesian pattern may maintain substantially uniform spatial sensing resolution across a curved surface having non-zero Gaussian curvature when the thin-film substrate is conformed thereto, whereas a conventional rectangular grid layout would produce non-uniform sensing coverage on such surfaces due to the geometric distortion introduced by the conforming process.

As used herein, the term 'substantially inelastic' when used to describe the thin-film substrate may refer to a substrate material that exhibits sufficiently low elongation at break and sufficiently high tensile modulus to maintain dimensional stability of printed sensor elements and electrical trace lines during and after conforming to a curved surface having non-zero Gaussian curvature. A substantially inelastic substrate as used herein does not undergo permanent dimensional changes or elastic deformation of a degree that would cause misalignment of printed components, compromise electrical connectivity between sensor elements and trace lines, or degrade sensing performance during the conforming process or operational use. Suitable substantially inelastic substrate materials include polyimide, polyethylene terephthalate, and polyethylene naphthalate, which exhibit elongation at break of less than about 100% and tensile modulus of greater than about 1 GPa.

As used herein, the term 'hub-and-petal architecture' may refer to the structural configuration of the thin-film substrate comprising a centralized hub region connected to a plurality of petal-shaped protrusions through a corresponding plurality of bridge regions. The hub region serves as the primary electrical routing and connection interface for the sensor array, while the petal-shaped protrusions extend radially outward from the hub and carry the sensor elements of the sensor array. The bridge regions function as flexible connection points that allow each petal-shaped protrusion to be deformed about the bridge and conformed to a curved surface while maintaining electrical continuity between the hub and the sensor elements disposed on the protrusion. The hub-and-petal architecture enables the substantially inelastic thin-film substrate to conform to non-developable surfaces having non-zero Gaussian curvature by distributing the mechanical deformation across the plurality of bridge regions rather than requiring uniform stretching of the substrate material.

Example 1. A conformal sensor device comprising: a thin-film substrate having a plurality of discrete regions and a plurality of patterned cutouts; a sensor array comprising a plurality of sensor elements disposed in a non-Cartesian pattern on the thin-film substrate, wherein the thin-film substrate having the plurality of sensor elements disposed thereon is configured to conform to a surface being substantially curved to provide substantially uniform sensing at the surface; and an integrated circuit electrically coupled to the plurality of sensor elements, wherein the integrated circuit is configured to analyze signals from the sensor array to determine one or more interactions with the surface.

Example 2. The conformal sensor device of example 1, wherein the integrated circuit comprises a processor and a memory comprising computer-readable instructions configured to trigger communication about the analyzed signals or interactions with the surface.

Example 3. The conformal sensor device of example 1, wherein: the thin-film substrate comprises a plurality of petal-shaped segments, and the plurality of patterned cutouts are wave-shaped or sawtooth-shaped along a perimeter of the plurality of petal-shaped segments.

Example 4. The conformal sensor device of example 3, wherein: the plurality of petal-shaped segments comprise active sensor areas and inactive sensor areas, and bending portions of the thin-film substrate to converge a distal end of each petal-shaped segment at an apex of the surface results in overlapping portions of the thin-film substrate to provide uniform sensing on the surface.

Example 5. The conformal sensor device of example 1, wherein: the substantially curved surface is a first surface having non-zero Gaussian curvature for receiving the thin-film substrate thereon; and the conformal sensor device further comprises a second surface as an outer layer of a material surrounding the thin-film substrate conformed to the first surface.

Example 6. The conformal sensor device of example 1, wherein the plurality of sensor elements is configured to measure at least one of: a pressure, a vibration, a shock, an acceleration, a stress, a force, a touch, humidity, a presence, damage, radiation, and temperature.

Example 7. The conformal sensor device of example 1, wherein the plurality of patterned cutouts comprise a cutout depth to minimize buckling deformation of the thin-film substrate and to minimize sensor performance degradation of one or more of the plurality of sensor elements when the conformal sensor device is curved about the surface.

Example 8. The conformal sensor device of example 1, wherein the thin-film substrate comprises a plurality of layers and at least a portion of the layers comprise cutouts in the plurality of patterned cutouts that enable adhesion of one layer to another of the plurality of layers.

Example 9. The conformal sensor device of example 1, wherein the conformal sensor device is a grip assessment device configured to measure grip characteristics of a user interacting with the substantially curved surface.

Example 10. The conformal sensor device of example 1, wherein the sensor array and a plurality of electrical trace lines coupling the sensor array to a ring bus are printed onto portions of one or more layers of the thin-film substrate, and a backing layer of the thin-film substrate comprises adhesive for coupling the thin-film substrate to a portion of the surface.

Example 11. The conformal sensor device of example 1, wherein the thin-film substrate further comprises: a plurality of petal-shaped segments, and a plurality of electrical trace lines coupling each of the plurality of sensor elements to a ring bus, the plurality of electrical trace lines being arranged substantially along a central meridian of each of the plurality of petal-shaped segments and within a predetermined clearance range defined from a perimeter of each respective petal-shaped segment.

Example 12. The conformal sensor device of example 1, wherein at least a portion of the thin-film substrate is composed of heat shrink material.

Example 13. The conformal sensor device of example 1, wherein each of the plurality of sensor elements comprises a discrete electrical element printed on the thin-film substrate having a non-square shape comprising at least one of: a triangular shape, an oval shape, a rectangular shape, or a polygonal shape other than rectangular.

Example 14. The conformal sensor device of example 1, wherein each of the plurality of sensor elements comprises a first discrete sensor element having a rectangular shape printed on the thin-film substrate and a second discrete sensor element having a rectangular shape printed on the thin-film substrate overlaid on the first discrete sensor element and oriented 90 degrees offset from a centerline of the first discrete sensor element.

Example 15. The conformal sensor device of example 1, wherein the sensor array is configured to distinguish finger orientation and identify digits of a user gripping at least a portion of the surface.

Example 16. The conformal sensor device of example 1, wherein the surface comprises an equatorial plane, wherein the sensor array extends radially outward from the equatorial plane, and wherein the integrated circuit is positioned at the equatorial plane and electrically coupled to the plurality of sensor elements of the sensor array.

Example 17. The conformal sensor device of example 1, wherein the sensor elements are arranged asymmetrically with variable densities to provide increased sensing resolution in targeted sensing areas where interaction frequency is expected to be higher relative to other areas of the surface.

Example 18. The conformal sensor device of example 1, wherein the integrated circuit is further configured to analyze signals from the sensor array to determine one or more of: a grip of a user gripping a portion of the substantially curved surface; a movement or series of movements of the substantially curved surface; and interactions with the portion of the substantially curved surface over a time period.

Example 19. An electronic device configured for installation in a ball, the electronic device comprising: a thin-film substrate having a hub and a plurality of protrusions each extending from one of a plurality of bridges coupled to a portion of the hub; a sensor array comprising a plurality of sensor elements disposed in a pattern over the plurality of protrusions, the plurality of sensor elements being divided amongst the plurality of protrusions and interconnected by a plurality of electrical trace lines emanating from each sensor element to another sensor element or to at least one ring bus; and an integrated circuit electrically coupled to the at least one ring bus, the integrated circuit comprising a processor and a memory comprising computer-readable instructions configured to analyze measurements from signals from the sensor array and trigger communication about the analyzed measurements.

Example 20. The electronic device of example 19, wherein the plurality of protrusions each comprise a first edge having a first set of cutouts extending along a first side of a periphery of the respective protrusion in the plurality of protrusions to a second edge having a second set of cutouts extending along a second side of the periphery of the respective protrusion.

Example 21. The electronic device of example 20, wherein the first set of cutouts and the second set of cutouts are wave-shaped or sawtooth-shaped.

Example 22. The electronic device of example 20, wherein the first set of cutouts and the second set of cutouts in the thin-film substrate are selected, based on a diameter or radius of curvature of the ball, with a cutout depth to minimize strain deformation of the thin-film substrate when curved about the ball.

Example 23. The electronic device of example 20, wherein each of the plurality of protrusions is petal-shaped and comprises a petal periphery with the first set of cutouts and the second set of cutouts in the thin-film substrate.

Example 24. The electronic device of example 19, wherein each of the protrusions is configured to cover a portion of a curved surface of a hemispherical shell when deformed about the respective bridges and coupled to the portion of the curved surface such that a distal end of each of the protrusions substantially covers an apex of the hemispherical shell.

Example 25. The electronic device of example 24, wherein the thin-film substrate comprises active sensor areas and inactive regions, and bending at the respective bridges to substantially cover the apex results in overlapping portions of the plurality of protrusions to provide substantially uniform sensing across a surface of the ball when the electronic device is installed in the ball.

Example 26. The electronic device of example 19, wherein each of the protrusions is configured to cover a portion of a curved surface of a prolate spheroid shape when deformed about the respective bridges and coupled to the portion of the curved surface such that a distal end of each of the protrusions substantially covers an apex of the prolate spheroid shape.

Example 27. The electronic device of example 19, wherein the plurality of sensor elements is configured to measure at least one of: a pressure, a vibration, a shock, an acceleration, a stress, a force, humidity, presence, damage, radiation, and temperature.

Example 28. The electronic device of example 19, further comprising: a second thin-film substrate having a second ring bus coupled to the integrated circuit and comprising a second plurality of protrusions and a second sensor array including a second plurality of sensor elements disposed in a pattern on the second plurality of protrusions, the second plurality of sensor elements being interconnected by a plurality of additional electrical trace lines emanating from each of the second plurality of sensor elements to another sensor element in the second sensor array or to the at least one ring bus, wherein the thin-film substrate is configured to wrap a first hemispherical portion of the ball, and the second thin-film substrate is configured to wrap a second hemispherical portion of the ball.

Example 29. The electronic device of example 28, wherein the sensor array and the second sensor array function together through the integrated circuit to analyze interaction with a surface of the ball to determine one or more of: a grip of a user gripping the ball, a movement or series of movements of the ball, and interactions with the ball over a time period.

Example 30. The electronic device of example 19, wherein the electronic device is configured to distinguish finger orientation and identify digits of a user gripping the ball.

The methods described herein and variations thereof can be embodied and/or implemented at least in part as a machine configured to receive a computer-readable medium storing computer-readable instructions. The instructions are executed by computer-executable components integrated with the system and one or more portions of the processors and/or computing devices described herein. The computer-readable medium can be stored on any suitable computer-readable media such as RAMs, ROMs, flash memory, EEPROMs, optical devices (e.g., CD or DVD), hard drives, floppy drives, or any suitable device. The computer-executable component is preferably a general or application-specific processor, but any suitable dedicated hardware or hardware/firmware combination can alternatively or additionally execute the instructions.

References in the specification to “one embodiment,” “an embodiment,” “an illustrative embodiment,” “some embodiments,” etc., indicate that the embodiment described may include a particular feature, structure, or characteristic, but every embodiment may or may not necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to effect such feature, structure, or characteristic in connection with other embodiments whether or not explicitly described.

As used in the description and claims, the singular form “a”, “an” and “the” include both singular and plural references unless the context clearly dictates otherwise. At times, the claims and disclosure may include terms such as “a plurality,” “one or more,” or “at least one;” however, the absence of such terms is not intended to mean, and should not be interpreted to mean, that a plurality is not conceived.

The term “about” or “approximately,” when used before a numerical designation or range (e.g., to define a length or pressure), indicates approximations which may vary by ( + ) or ( - ) 5%, 1% or 0.1%. All numerical ranges provided herein are inclusive of the stated start and end numbers. The term “substantially” indicates mostly (i.e., greater than 50%) or essentially all of a device, substance, or composition.

As used herein, the term “comprising” or “comprises” is intended to mean that the devices, systems, and methods include the recited elements, and may additionally include any other elements. “Consisting essentially of” shall mean that the devices, systems, and methods include the recited elements and exclude other elements of essential significance to the combination for the stated purpose. Thus, a system or method consisting essentially of the elements as defined herein would not exclude other materials, features, or steps that do not materially affect the basic and novel characteristic(s) of the claimed disclosure. “Consisting of” shall mean that the devices, systems, and methods include the recited elements and exclude anything more than a trivial or inconsequential element or step. Embodiments defined by each of these transitional terms are within the scope of this disclosure.

The examples and illustrations included herein show, by way of illustration and not of limitation, specific embodiments in which the subject matter may be practiced. Other embodiments may be utilized and derived therefrom, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is in fact disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A conformal sensor device comprising:
a thin-film substrate having a plurality of discrete regions and a plurality of patterned cutouts;
a sensor array comprising a plurality of sensor elements disposed in a non-Cartesian pattern on the thin-film substrate, wherein the thin-film substrate having the plurality of sensor elements disposed thereon is configured to conform to a surface being substantially curved to provide substantially uniform sensing at the surface; and
an integrated circuit electrically coupled to the plurality of sensor elements, wherein the integrated circuit is configured to analyze signals from the sensor array to determine one or more interactions with the surface.

2. The conformal sensor device of claim 1, wherein the integrated circuit comprises a processor and a memory comprising computer-readable instructions configured to trigger communication about the analyzed signals or interactions with the surface.

3. The conformal sensor device of claim 1, wherein:
the thin-film substrate comprises a plurality of petal-shaped segments, and
the plurality of patterned cutouts are wave-shaped or sawtooth-shaped along a perimeter of the plurality of petal-shaped segments.

4. The conformal sensor device of claim 3, wherein:
the plurality of petal-shaped segments comprise active sensor areas and inactive sensor areas, and
bending portions of the thin-film substrate to converge a distal end of each petal-shaped segment at an apex of the surface results in overlapping portions of the thin-film substrate to provide uniform sensing on the surface.

5. The conformal sensor device of claim 1, wherein:
the substantially curved surface is a first surface having non-zero Gaussian curvature for receiving the thin-film substrate thereon; and
the conformal sensor device further comprises a second surface as an outer layer of a material surrounding the thin-film substrate conformed to the first surface.

6. The conformal sensor device of claim 1, wherein the plurality of sensor elements is configured to measure at least one of: a pressure, a vibration, a shock, an acceleration, a stress, a force, a touch, humidity, a presence, damage, radiation, and temperature.

7. The conformal sensor device of claim 1, wherein the plurality of patterned cutouts comprise a cutout depth to minimize buckling deformation of the thin-film substrate and to minimize sensor performance degradation of one or more of the plurality of sensor elements when the conformal sensor device is curved about the surface.

8. The conformal sensor device of claim 1, wherein the thin-film substrate comprises a plurality of layers and at least a portion of the layers comprise cutouts in the plurality of patterned cutouts that enable adhesion of one layer to another of the plurality of layers.

9. The conformal sensor device of claim 1, wherein the conformal sensor device is a grip assessment device configured to measure grip characteristics of a user interacting with the substantially curved surface.

10. The conformal sensor device of claim 1, wherein the sensor array and a plurality of electrical trace lines coupling the sensor array to a ring bus are printed onto portions of one or more layers of the thin-film substrate, and a backing layer of the thin-film substrate comprises adhesive for coupling the thin-film substrate to a portion of the surface.

11. The conformal sensor device of claim 1, wherein the thin-film substrate further comprises:
a plurality of petal-shaped segments, and
a plurality of electrical trace lines coupling each of the plurality of sensor elements to a ring bus, the plurality of electrical trace lines being arranged substantially along a central meridian of each of the plurality of petal-shaped segments and within a predetermined clearance range defined from a perimeter of each respective petal-shaped segment.

12. The conformal sensor device of claim 1, wherein at least a portion of the thin-film substrate is composed of heat shrink material.

13. The conformal sensor device of claim 1, wherein each of the plurality of sensor elements comprises a discrete electrical element printed on the thin-film substrate having a non-square shape comprising at least one of: a triangular shape, an oval shape, a rectangular shape, or a polygonal shape other than rectangular.

14. The conformal sensor device of claim 1, wherein each of the plurality of sensor elements comprises a first discrete sensor element having a rectangular shape printed on the thin-film substrate and a second discrete sensor element having a rectangular shape printed on the thin-film substrate overlaid on the first discrete sensor element and oriented 90 degrees offset from a centerline of the first discrete sensor element.

15. The conformal sensor device of claim 1, wherein the sensor array is configured to distinguish finger orientation and identify digits of a user gripping at least a portion of the surface.

16. The conformal sensor device of claim 1, wherein the surface comprises an equatorial plane, wherein the sensor array extends radially outward from the equatorial plane, and wherein the integrated circuit is positioned at the equatorial plane and electrically coupled to the plurality of sensor elements of the sensor array.

17. The conformal sensor device of claim 1, wherein the sensor elements are arranged asymmetrically with variable densities to provide increased sensing resolution in targeted sensing areas where interaction frequency is expected to be higher relative to other areas of the surface.

18. The conformal sensor device of claim 1, wherein the integrated circuit is further configured to analyze signals from the sensor array to determine one or more of:
a grip of a user gripping a portion of the substantially curved surface;
a movement or series of movements of the substantially curved surface; and
interactions with the portion of the substantially curved surface over a time period.

19. An electronic device configured for installation in a ball, the electronic device comprising:
a thin-film substrate having a hub and a plurality of protrusions each extending from one of a plurality of bridges coupled to a portion of the hub;
a sensor array comprising a plurality of sensor elements disposed in a pattern over the plurality of protrusions, the plurality of sensor elements being divided amongst the plurality of protrusions and interconnected by a plurality of electrical trace lines emanating from each sensor element to another sensor element or to at least one ring bus; and
an integrated circuit electrically coupled to the at least one ring bus, the integrated circuit comprising a processor and a memory comprising computer-readable instructions configured to analyze measurements from signals from the sensor array and trigger communication about the analyzed measurements.

20. The electronic device of claim 19, wherein the plurality of protrusions each comprise a first edge having a first set of cutouts extending along a first side of a periphery of the respective protrusion in the plurality of protrusions to a second edge having a second set of cutouts extending along a second side of the periphery of the respective protrusion.

21. The electronic device of claim 20, wherein the first set of cutouts and the second set of cutouts are wave-shaped or sawtooth-shaped.

22. The electronic device of claim 20, wherein the first set of cutouts and the second set of cutouts in the thin-film substrate are selected, based on a diameter or radius of curvature of the ball, with a cutout depth to minimize strain deformation of the thin-film substrate when curved about the ball.

23. The electronic device of claim 20, wherein each of the plurality of protrusions is petal-shaped and comprises a petal periphery with the first set of cutouts and the second set of cutouts in the thin-film substrate.

24. The electronic device of claim 19, wherein each of the protrusions is configured to cover a portion of a curved surface of a hemispherical shell when deformed about the respective bridges and coupled to the portion of the curved surface such that a distal end of each of the protrusions substantially covers an apex of the hemispherical shell.

25. The electronic device of claim 24, wherein the thin-film substrate comprises active sensor areas and inactive regions, and bending at the respective bridges to substantially cover the apex results in overlapping portions of the plurality of protrusions to provide substantially uniform sensing across a surface of the ball when the electronic device is installed in the ball.

26. The electronic device of claim 19, wherein each of the protrusions is configured to cover a portion of a curved surface of a prolate spheroid shape when deformed about the respective bridges and coupled to the portion of the curved surface such that a distal end of each of the protrusions substantially covers an apex of the prolate spheroid shape.

27. The electronic device of claim 19, wherein the plurality of sensor elements is configured to measure at least one of: a pressure, a vibration, a shock, an acceleration, a stress, a force, humidity, presence, damage, radiation, and temperature.

28. The electronic device of claim 19, further comprising:
a second thin-film substrate having a second ring bus coupled to the integrated circuit and comprising a second plurality of protrusions and a second sensor array including a second plurality of sensor elements disposed in a pattern on the second plurality of protrusions, the second plurality of sensor elements being interconnected by a plurality of additional electrical trace lines emanating from each of the second plurality of sensor elements to another sensor element in the second sensor array or to the at least one ring bus, wherein
the thin-film substrate is configured to wrap a first hemispherical portion of the ball, and
the second thin-film substrate is configured to wrap a second hemispherical portion of the ball.

29. The electronic device of claim 28, wherein the sensor array and the second sensor array function together through the integrated circuit to analyze interaction with a surface of the ball to determine one or more of:
a grip of a user gripping the ball,
a movement or series of movements of the ball, and
interactions with the ball over a time period.

30. The electronic device of claim 19, wherein the electronic device is configured to distinguish finger orientation and identify digits of a user gripping the ball.
